(19) United States
(12) Patent Application Publication
(10) Pub. No.: US 2019/0378597 A1
(43) Pub. Date: Dec. 12, 2019

(54) NOVEL HEALTHCARE DELIVERY, TREATMENT, AND PAYMENT MODEL FOR SPECIALTY DRUGS

(71) Applicant: Vijay Ramakrishnan, Palo Alto, CA (US)

(72) Inventor: Vijay Ramakrishnan, Palo Alto, CA (US)

(21) Appl. No.: 15/360,799

(22) Filed: Nov. 23, 2016

(51) Int. Cl. G16H 20/10; G16H 50/70; G16H 10/60; G06Q 40/08

(52) U.S. Cl. CPC G16H 20/10; G06Q 40/08; G16H 10/60; G16H 50/70

(57) ABSTRACT

The present disclosure provides methods, processes, reagents and kits for novel healthcare delivery, treatment and payment, particularly for achieving better economic outcomes in patients treated with specialty drugs. Methods, processes, and kits provide a number of uses, including, for providing assurance, both efficacy and financial, theragnostic-guidance, for providing therapeutic appropriateness, therapeutic guidance, therapeutic effectiveness, and selection of alternative therapeutic strategies, and providing product differentiation and presumably market enrichment for a specialty drug, for authorizing payment and delivering the drug directly from a pharmaceutical company to a patient or her specialty physician through a novel healthcare delivery platform. Medical records and databases using these strategies are also provided.

### BACKGROUND

Specialty drugs are widely administered to treat a wide variety of diseases and disorders including, e.g., oncology indications, autoimmune diseases, inflammatory disorders, microbial infections, rare diseases, and ultra-rare diseases. Achieving excellent clinical outcome, i.e., excellent therapeutic response or clinical remission of the disease, in a subject treated with a specialty drug leads to significantly better economic outcomes for payers, employers, pension funds, and patients. An effective and practical treatment model which could provide information as to whether an individual patient will or will not be responsive to a specialty drug therapy would be desirable for several reasons, including avoidance of delays in alternative treatments, elimination of exposure to adverse effects, and reduction of unnecessary treatment expenses. In addition, achieving efficiency in the healthcare delivery process of a specialty drug will result in significant reduction of overall healthcare spending. The methods, processes, reagents, and kits presented herein address these inefficiencies.

### SUMMARY OF THE INVENTION

Methods and processes of healthcare delivery and treatment are provided, particularly for achieving better economic outcomes in patients treated with specialty drugs. Methods, processes, and kits provide several uses, e.g., for providing therapeutic guidance, for providing therapeutic efficacy assurance, for providing product differentiation of a specialty drug, for delivering drugs from a pharmaceutical company to patient through a novel healthcare delivery platform, etc.

The present invention is based, in part, upon the observation that achieving better clinical outcomes to specialty drug treatment in mechanistically stratified or targeted subset(s) of patient(s) can lead to better economic outcomes for payers, employers, pension funds, patients, dependents, and families. Theragnostic guidance can be a key factor in providing therapeutic efficacy assurance to stakeholders. The present invention is based upon the additional observation that major inefficiencies in healthcare supply chain and delivery model can be addressed by integrating and streamlining key processes pertaining to healthcare delivery and treatment.

The present inventions provide several novel aspects of healthcare delivery, treatment, and payment model for specialty drugs. The first is a method providing assurance for a specialty drug treatment, comprising selecting a specialty drug by a theragnostic evaluation of a patient for treatment of a chronic disease or disorder; and providing efficacy assurance or financial assurance. In preferred embodiments, the method provides both efficacy assurance and financial assurance; or the chronic disease or disorder is subject to theragnostic evaluation, specialty drug treatment, and efficacy assurance. In other embodiments, the method further comprises: selecting the specialty drug from a panel of available drugs in a drug formulary; treating the patient with an appropriate specialty drug; and/or achieving better than about 70%, e.g., 80, 90, or 95%, patient therapeutic adherence. In another, the method is applied to a plurality of individual patients, including a disease population or subset(s) of patients. In other embodiments, the theragnostic evaluation provides, typically prospectively: therapeutic appropriateness in the patient or group of patients; therapeutic guidance in the patient or group of patients; and/or therapeutic effectiveness in the patient or group of patients; often also with selection of an alternative therapeutic strategy, e.g., if there are contraindications (e.g., pharmacological or mechanism problems in the patient) or the first strategy fails. Often, the theragnostic will further be directed to further provide efficacy assurance, financial assurance, prior authorization or payment approval, and/or providing guidelines in developing a disease specific drug formulary. In other embodiments, the disease or disorder is an oncology indication, an autoimmune disease, an inflammatory disorder, a rare disease, or a microbial infection; the oncology indication is B-cell non-Hodgkin's lymphoma (B-NHL); the autoimmune disease is rheumatoid arthritis; the inflammatory disorder is relapsing-remitting multiple sclerosis; or the microbial infection is hepatitis C viral infection.

In other preferred embodiments, the theragnostic evaluation further: stratifies a disease population into one or more distinct subsets based on immunological subtype(s); stratifies a disease population into one or more distinct subsets based on disease severity or some other relevant feature; achieves significant therapeutic response in one or more subtypes of disease by administering a specialty drug; achieves significant therapeutic response in a subject categorized according to a set of immunological subtypes by administering a specialty drug; achieves significant therapeutic response in multiple subset(s) of the disease population; and/or includes evaluation of responsiveness to drug during treatment.

The efficacy assurance may be provided: for a specialty drug for the treatment of a disease, disorder, or cancer; and/or by using a theragnostic evaluation comprising use of a biomarker, diagnostic procedure, companion diagnostic procedure, or combination thereof. In yet other embodiments, the efficacy assurance is provided to: an eligible patient selected from a disease population; an eligible subset of patients selected from a disease population; or all eligible patients selected from a disease population. In other embodiments, the therapeutic efficacy assurance is provided to an eligible patient, payer, or employer, e.g., where the payer is a private payer or government payer.

The financial assurance may be provided to the eligible patient, payer, or employer; and/or the financial assurance may involve: full or partial money-back guarantee of co-insurance to the eligible patient; or full or partial money-back guarantee to the payer or the employer who pays for the specialty drug.

In yet another embodiment of the method, the specialty drug is: approved by a disease specialist; intended for treating a chronic disease or disorder, autoimmune disease, inflammatory disorder, a rare disease, a cancer indication, or microbial infection; further delivered or dispensed for administration to the patient; a biotech product or biologic; an oral or injectable formulation; or subject to post approval surveillance, e.g., risk evaluation and mitigation strategies, from drug manufacturer(s). The invention further encompasses an entity, e.g., a pharmacy, which may be a specialty pharmacy; a pharmacy benefits management entity; an employer or insurance entity, including a health insurer, a commercial health insurer, or a public/government health insurer; a healthcare provider entity, including a clinic, hospital, outpatient facility, specialty clinic, or physician's office; a pharmaceutical or drug manufacturing company; or a diagnostic company, which directly or indirectly uses or pays for these described methods.

In another aspect of the inventions, a method is provided which allows assurance-based prior authorization of a specialty drug, comprising using a theragnostic evaluation of a patient for treatment of a chronic disease or disorder; and making a decision on prior authorization for payment for the specialty drug. In preferred embodiments, the method further comprises: selecting the specialty drug from a panel of available drugs in a drug formulary; dispensing the specialty drug by a specialty pharmacy to the patient, and/or delivering the specialty drug to the patient. In other embodiments, the theragnostic evaluation provides, typically prospectively: therapeutic appropriateness; therapeutic guidance; and/or therapeutic effectiveness; often also with selection of an alternative therapeutic strategy, e.g., if there are contraindications (e.g., pharmacological or mechanism problems in the patient) or the first strategy fails; and/or support for an assurance-based prior authorization decision, e.g., based on efficacy assurance or financial assurance. Often, the theragnostic will further be directed to further provide efficacy assurance, financial assurance, prior authorization or payment approval, and/or providing guidelines in developing a disease specific drug formulary. Alternatively, the assurance-based prior authorization is provided: for a specialty drug for the treatment of a disease, disorder, or cancer; by using a theragnostic evaluation comprising use of a biomarker, diagnostic procedure, companion diagnostic procedure, or combination thereof; for bundles of like patients having similar, or functionally equivalent, theragnostic measures, such that such bundles of patients are subject to a like decision; for a bundle of like patients numbering at least about 2000, e.g., 4000, or 8000 individual patients; or where the prior authorization covers at least about 40%, e.g., 60% or 80%, of the specialty drug cost as compared to the Medicare non-negotiated cost.

Or, the prior authorization may involve: approval of the specialty drug that was originally prescribed by a disease specialist of the patient; or substitution of the specialty drug that was originally prescribed by a disease specialist of the patient with an alternate specialty drug or non-specialty drug by a Disease and Therapy Management specialist. Similarly, this aspect of the invention also provides an entity, e.g., a pharmacy, including a specialty pharmacy; a pharmacy benefits management entity; an employer or insurance entity, including a health insurer, a commercial health insurer, or a public/government health insurer; a healthcare provider entity, including a clinic, hospital, outpatient facility, specialty clinic, or physician's office; a pharmaceutical or drug manufacturing company; or a diagnostic or related healthcare service company; which directly or indirectly uses or pays for these described methods.

In another aspect of the invention, a method is provided generating a specialty drug formulary for treating a specific disease, comprising identifying specialty drugs that are highly efficacious in distinct subsets of patients based on a set of theragnostic evaluation(s). In preferred embodiments, the methods further comprise: including the specialty drugs in the formulary; treating a patient or subset of patients with an appropriately-matched specialty drug to achieve excellent therapeutic efficacy; and/or providing efficacy assurance for a specialty drug that is chosen for treatment from the formulary. Another embodiment of the invention is the resulting formulary.

In other embodiments, the drug formulary is generated: to include a specialty drug for the treatment of a disease, disorder, or cancer; or by using a theragnostic evaluation comprising use of a biomarker, diagnostic procedure, companion diagnostic procedure, or combination thereof, including assignment by genotypic evaluation. This aspect of the invention further provides an entity, e.g., a pharmacy, including a specialty pharmacy; a pharmacy benefits management entity; an employer or insurance entity, including a health insurer, a commercial health insurer, or a public/government health insurer; a healthcare provider entity, including a clinic, hospital, outpatient facility, specialty clinic, or physician's office; a pharmaceutical or drug manufacturing company; or a diagnostic or related healthcare service company; which directly or indirectly uses the drug formulary, or pays for the specialty drug that is chosen for treatment from the formulary.

Another aspect of the invention provides a method delivering a specialty drug and/or treating a patient with a specialty drug, the method achieving an improvement derived from a theragnostic evaluation, patient therapeutic adherence, or pricing change (e.g., ability to negotiate and reduce drug price with a biopharmaceutical company), the improvement in: distribution and/or delivery efficiency; a priori matching of appropriate specialty drug to the individual patient; treatment efficiency; patient therapeutic adherence efficiency; product differentiation for a specialty drug in a disease indication; or market enrichment for a specialty drug in a disease indication. In some preferred embodiments, components of the distribution and/or treatment method include: a prescription drug plan; a specialty drug formulary; a specialty pharmacy; a theragnostic facility providing disease-specific theragnostic evaluation; or a disease and therapy management care specializing in a specific disease.

In some embodiments, at least one of the components of the specialty drug distribution and/or treatment method uses a telehealth architecture, e.g., connecting remote locations through telephone or data link connections. In other embodiments, the disease and/or therapy management care is through telehealth architecture, and: the care is provided by disease-specific specialty doctor or specialty nurse; the disease is an oncology indication, autoimmune disease, inflammatory disorder, or microbial infection; the oncology indication is B-cell non-Hodgkin's lymphoma (B-NHL); the autoimmune disease is rheumatoid arthritis; the inflammatory disorder is relapsing-remitting multiple sclerosis; or the microbial infection is hepatitis C viral infection.

In yet other embodiments, the disease and/or therapy management care involves: approval of the specialty drug that was originally prescribed by the disease specialist of the patient; or substitution of the specialty drug that was originally prescribed by a disease specialist of the patient with an alternate specialty drug or non-specialty drug, e.g., properly approved with assistance, by a Disease and Therapy Management specialist. This aspect of the invention also provides an entity, e.g., a pharmacy, including a specialty pharmacy; a pharmacy benefits management entity; an employer or insurance entity, including a health insurer, a commercial health insurer or a public/government health insurer; a healthcare provider entity, including a clinic, hospital, outpatient facility, specialty clinic, or physician's office; a pharmaceutical or drug manufacturing company; or a diagnostic or related healthcare service company; which directly or indirectly uses or pays for these methods.

Another aspect of the invention provides a method providing assurance based on theragnostic evaluation of a patient for specialty drug distribution and/or treatment, wherein the patient is subjected to theragnostic evaluation to select and administer an appropriate specialty drug. In certain embodiments, the method is applied to a plurality of patients. In some preferred embodiments, the methods further comprise: selecting an appropriate specialty drug matched for the patient or subset of patients to achieve better treatment outcomes; evaluating a disease population by theragnostic evaluation to stratify into distinct subsets, and administering an appropriate specialty drug in that subset to achieve better treatment outcomes; using theragnostic evaluation in guiding therapeutic dosing and/or scheduling during treatment; using theragnostic indications for evaluating therapeutic outcome(s) during treatment cycle; or selecting an alternate specialty drug for the patient at the end of the treatment cycle, if the patient failed to respond to the treatment.

In other preferred embodiments, the distribution and/or treatment are provided: for a specialty drug for the treatment of a disease, disorder, or cancer; or by using a theragnostic evaluation comprising use of a biomarker, diagnostic procedure, companion diagnostic procedure, or combination thereof. In other embodiments, an entity, e.g., a pharmacy, including a specialty pharmacy; a pharmacy benefits management entity; an employer or insurance entity, including a health insurer, a commercial health insurer, or a public/government health insurer; a healthcare provider entity, including a clinic, hospital, outpatient facility, specialty clinic, or physician's office; a pharmaceutical or drug manufacturing company; or a diagnostic or healthcare services company; which directly or indirectly uses or pays for these methods.

In yet another aspect of the inventions is provided methods achieving patient therapeutic adherence for a specialty drug treatment, comprising: selecting a specialty drug by a theragnostic evaluation of a patient for treatment of a specific disease or disorder; and/or providing efficacy assurance, e.g., therapeutic or financial assurance. Either or both promote patient adherence, or compliance, with treatment protocols by improving treatment outcomes and/or decreasing downside risk. These may be combined with additional aspects of patient education, among other factors affecting patient adherence. In certain preferred embodiments, the methods further comprise steps incorporating a telehealth architecture, e.g., in disease and therapy management team or monitoring, telepharmacy, financial approval and payment exchange, or patient therapy adherence monitoring. In other embodiments, the methods further comprise location-based authentication or certification; or time-dependent authentication or certification, e.g., of compliance with therapy instructions, typically time logs of drug administration or dosing.

In other embodiments, an entity, e.g., a pharmacy, including a specialty pharmacy; a pharmacy benefits management entity; an employer or insurance entity, including a health insurer, a commercial health insurer, or a public/government health insurer; a healthcare provider entity, including a clinic, hospital, outpatient facility, specialty clinic, or physician's office; a pharmaceutical or drug manufacturing company; or a diagnostic or healthcare services company; which directly or indirectly uses or pays for these methods.

In some embodiments, the integrated model, herein referred to as Klaritos™ platform, includes a specialty drug formulary, specialty pharmacy, theragnostic laboratories, disease and therapy management care. In some aspects of the invention, all the components of the healthcare supply chain and delivery model operate as one integrated system. In some embodiments, all the components of the healthcare supply chain and delivery model use telehealth medium. In other embodiment, the specialty pharmacy is a telepharmacy through which a patient can interact with a specialty pharmacist. In other embodiments, an in-house, disease-specific, specialty physician(s) and specialty nurse(s) operate the disease and therapy management care. In yet another embodiment, the specialty physician(s) and specialty nurse(s) perform the roles of a healthcare provider. In other embodiments, examples of such specialty disease include rheumatoid arthritis, multiple sclerosis, cancers such as breast cancer, colorectal cancer, etc. In some other embodiments, these functions are integrated to theragnostic laboratories in regards to selection of a therapy, selection of a patient for treatment with an appropriate therapy, methods of treatment, differential dosing, differential dosing schedule, differential dosing frequency, etc.

In some embodiments, the healthcare delivery and treatment model is a platform through which all stakeholders are efficiently connected to achieve maximum efficiency in regards to: delivery, care, clinical and economic outcomes, and payments. In another embodiment, external pharmacy benefit management companies (PBMs), specialty pharmacies, distributors, hospitals, specialty clinics or the specialty physicians are not involved in fixing the drug price. In one embodiment, the stakeholders of the platform include: pharmaceutical companies, theragnostic providers, diagnostic providers, healthcare providers, patients, and payers (FIG. 1). In another embodiment, this platform is an essential cog in the delivery and treatment model by providing significant proprietary advantages to stakeholders. In another embodiment, the platform provides an efficient market place for pharma companies and diagnostic companies to offer their products and services to payers and patients. In yet another embodiment, payers are insurance companies, employers, government payers. In yet another embodiment, patients are payers.

In some embodiments, the healthcare delivery and treatment platform specializes in specific diseases. In one embodiment, the disease or disorder is selected from the group consisting of oncology indications, autoimmune diseases, inflammatory disorders, microbial infections, rare diseases, and ultra-rare diseases. In one instance, the disease is rheumatoid arthritis. In another instance, it is relapsing-remitting multiple sclerosis. In yet another instance, it is breast cancer. In another instance, it is B-cell chronic lymphocytic leukemia (B-CLL). Accordingly, in one embodiment, the platform can provide therapeutic guidance by identifying and selecting a priori several of the marketed specialty drugs for the entire disease population. In another embodiment, the platform can provide therapeutic guidance by identifying and selecting a priori several of the marketed specialty drugs for 50-70% or more of the disease population. In other embodiments, the platform can provide therapeutic guidance by identifying and selecting a priori only one or two of the marketed specialty drugs targeting 10-20% or 20-40% of the disease population. In one embodiment, the disease is rheumatoid arthritis. In another embodiment, the disease is relapsing-remitting multiple sclerosis. In some embodiments, the specialty drug is an antibody therapy. In certain embodiments, the anti-CD20 antibody is rituximab. In other embodiments, the specialty drug is a small molecule therapy. In yet other embodiments, the specialty drug is an intravenous chemotherapy.

In some embodiments, the healthcare delivery and treatment platform adopts different kinds of diagnostic, biomarker tests and stratification platforms as part of its proprietary theragnostic guidance. In one embodiment, the specialty drug has an existing companion diagnostic product, approved by regulatory agencies such as FDA and EMEA. In another embodiment, the specialty drug does not have a companion diagnostic product but has an independent diagnostic product approved by a regulatory agency for that drug in a particular indication. In another embodiment, the specialty drug has an existing CLIA-certified diagnostic or biomarker product. In some embodiments, the specialty drug is a targeted therapy, e.g., an antibody therapy, e.g., anti-CD52 antibody, but does not have any approved biomarker tests.

In some aspects of the invention, all the financial transactions KlariPay™ occur electronically. In one embodiment, KlariPay is a two-way exchange of assets and such assets include specialty drug, theragnostic guidance, and money. In another embodiment, KlariPay is considered a securities lending and repurchase agreement. In another embodiment, the financial transaction is instant between a payer and an assurance company through KlariPay. An external entity, e.g., re-insurance (assurance) company may provide just the financial assurance whereas the therapeutic efficacy assurance is provided by KlariPay. Alternatively, both efficacy and financial assurances are provided by KlariPay. In an alternate embodiment, a pharmaceutical company and (or) a financial risk assurance (re-insurance) company may involve as stakeholders in this transaction. When a pharmaceutical company or a financial risk assurance (re-insurance) company is not part of such assurance, such assurance is provided by KlariPay to payers (FIG. 3, 4).

In some aspects of the invention, methods are provided to develop a proprietary therapeutic efficacy assurance, KlariPay™. KlariPay is the assurance platform that provides both efficacy and financial assurances to payers. In an embodiment, KlariPay platform is part of the telehealth platform, e.g., Klaritos platform. In one embodiment, this solution integrates the benefits of (a) theragnostics, (b) specialty pharmacy operations, (c) pharmacy benefit management, (d) disease and therapy management care (FIG. 1). In another embodiment, payment for the specialty drug is performance-based and tied to the therapeutic efficacy in a patient. In yet another embodiment, the payment is ensured once pre-defined clinical or therapeutic response criteria are achieved. In another embodiment, the payment is made on a provisional basis by the payer immediately upon dispensing the drug. In some embodiments, payers mean government or private payers, employers, pension funds, and patients. In other embodiments, the specialty drug company agrees to pay back the payment received from payers via KlariPay, minus the applicable costs for goods and services rendered by the specialty drug company, and the theragnostic provider/specialty pharmacy provider, if the patient does not achieve the intended clinical response; e.g., remission or excellent response or such pre-defined criteria; depending on the disease and stage of the disease (FIG. 4). In an alternate embodiment, the specialty drug company (e.g., pharmaceutical company) is not involved in providing such assurance, and it is exclusively provided by an assurance company to payers. In another embodiment, if the patient achieves clinical remission (or excellent clinical response), payer(s) agree to pay KlariPay additional price for the specialty drug as a performance incentive. In one instance, e.g., if the annual drug price is $50,000, then the payer agrees to pay an additional $15,000 (30%) to KlariPay. In another instance, specialty drug manufacturer (pharmaceutical company) and the KlariPay provider may split this 30% payment, e.g., in two equal halves. In an alternative embodiment, assuming the patient is in remission during the second year after the administration of the specialty drug, and no additional treatment is provided to the patient during this period, the payer will pay an additional two-thirds of the drug cost to KlariPay. Specialty drug manufacturer and the theragnostic guidance provider and the specialty pharmacy provider may split this payment, e.g., in two equal halves; theragnostic guidance provider and the specialty pharmacy provider may form components of one entity, e.g., a prescription drug plan, an assurance company, a PBM company, a payer. The additional payment is for the significant avoidance of medical and pharmacy cost(s) the payer might have accrued otherwise.

In some aspects of the invention, methods are provided for a novel commercialization approach, termed herein as theragnostics-guided pull through strategy, to commercialize specialty drugs through this healthcare supply chain and delivery model. In one embodiment, such a commercialized drug is expected to differentiate itself from other commercially available IP-protected drugs, and other biosimilars and small molecule generic drugs in regards to efficacy, safety and toxicity profiles. In one instance, even if the drug is administered only in 25% of the market where it is known to work exceptionally well, because of the market enrichment, this delivery model can conceivably increase net sales of the drug, possibly 2-fold, and in some instances 3-5 fold, by bringing more patients who are eligible from within this stratified segment. In another embodiment, payers will approve this specialty drug because of the therapeutic efficacy assurance provided by KlariPay. In yet another embodiment, though currently payers may approve a specialty drug as a third or fourth-line therapy in an indication, e.g., rheumatoid arthritis, because of the assured therapeutic efficacy and the KlariPay payment model, they may approve this as an earlier first or second-line therapy. In another embodiment, this leads to market enrichment of a specialty drug through targeted drug use, significantly improved patient therapeutic adherence as well as therapy guidelines adherence (TGA), and may improve market penetration of one among multiple biosimilars.

In some embodiments of theragnostics, the methods may comprise, e.g., genotyping or phenotyping the individual for one or more genotypic polymorphisms to obtain a result; genotyping point mutations or gene deletions to obtain a result; determining depletion of specific cell population in a subject as a function of treatment response, disease remission, disease relapse, etc.; determining re-population of a specific cell population as a function of treatment response, disease remission, disease relapse, etc.; stratifying a disease, e.g., rheumatoid arthritis, into distinct subsets of diseases or into categories of subsets based on disease severity; stratifying patients based on one or two functional polymorphisms that are relevant to the mechanism of the action of a drug, and in one instance these polymorphisms are FcGR-3A V/F158 and FcGR-2A H/R131 polymorphisms, and the mechanism of action Is antibody-mediated cellular cytotoxicity (ADCC). In yet another embodiment, ivacaftor (Kalydeco®) is administered to treat cystic fibrosis in patients who carry a genetic mutation, G551 D.

In some embodiments of theragnostics, the decision to treat a disease with a specialty drug is a function of the mechanism of action of the drug. In one embodiment, the specialty drug is rituximab antibody therapy and the mechanism of action is antibody-dependent cellular cytotoxicity (ADCC), and the neoplastic disease is B-cell non-Hodgkin's lymphoma (B-NHL), e.g., follicular lymphoma. In another embodiment, the Cobas® KRAS Mutation Test is used as an aid in the identification of colorectal patients for whom treatment with Erbitux® (cetuximab) may be indicated if mutations are not detected.

In some embodiments of theragnostics, the decision to treat a disease with a specialty drug is a function of the pathophysiology of the disease as stratified based on immunologically defined subtypes of disease and disease severity. In one embodiment, the disease is rheumatoid arthritis (RA), and subtypes of RA include: IgG RF⁺ or IgA RF⁺ (rheumatoid factor of IgG or IgA subtypes); IgG ACPA or IgA ACPA⁺ (anti-citrullinated peptide antibodies of IgG or IgA subtypes); fibrinogen-induced arthritis (FIA) or collagen-induced arthritis (CIA) in defined subsets of populations, e.g., as characterized by the FcGR3A (V/F¹⁵⁸) and FcGR2A (H/R¹³¹) polymorphisms.

In some embodiments, theragnostics is used to select a specialty drug for treatment a priori. In other embodiments, theragnostic guidance is provided to the patient during the treatment in regards to differential dosing, differential dosing schedule, differential dosing frequency, disease remission, disease relapse, etc. In yet other embodiments, theragnostics provides a treatment decision to discontinue the current therapy and select an alternate therapy. Such reasons for discontinuations include: in one instance, the subject may develop serious side effects to the current therapy; and in other instances, the drug is no longer efficacious in the subject; and in yet other instances, the subject may have developed resistant mutation(s) that makes the drug ineffective.

In other embodiments of theragnostics, a specialty drug is specifically chosen from a panel of therapies available in a drug formulary or prescription drug plan, including from a panel of specialty drugs, for treatment. In other embodiments, a patient or subset(s) of patients is specifically chosen for a specialty drug treatment; and in other embodiments of theragnostics, based on the understanding that the patients will respond poorly to the therapy, a patient or subset(s) of patients is specifically not chosen for a specialty drug treatment.

In other embodiments of theragnostics, the specialty drug chosen for treatment is an induction therapy. In other embodiment, the specialty drug is a maintenance therapy. In other embodiments, the specialty drug can be used as a monotherapy in both induction and maintenance therapy settings. In other embodiments, the specialty drug can be used as one of the therapies of a combination therapy in both induction and maintenance therapy settings. Accordingly, e.g., the specialty drug can be an antibody therapy, and one such therapy is rituximab for the treatment of B-NHL. For instance, an antibody therapy and chemotherapy can form the combination therapy.

In some aspects of the invention, methods are provided for therapeutic guidance based on theragnostics. This includes selection of a specialty drug from a panel of marketed specialty drugs for a subject or a patient subset; selection of a treatment regimen (single course versus maintenance therapy; monotherapy versus combination therapy; or simply a ‘watch and wait’ regimen in the case of B-NHL). In some embodiments, the methods comprise mechanism-driven theragnostic methods: (a) based on the mechanism of action by which the drug exerts therapeutic response in an individual or in individuals having the desired genetic or immunological makeup, and by determining whether the said patient will then respond to that therapy or not; (b) based on the disease severity mechanisms patient population can be stratified and the appropriate specialty drugs are then administered to achieve better clinical responses, preferably clinical remission. In some other embodiments, the methods comprise continued, systematic monitoring of disease remission and relapse patterns during the course of administration—to ascertain how well the drug is working (or not working) in a given subject or vice versa; and/or when to administer the next course of therapy (e.g., as-needed versus fixed time intervals).

In yet a further embodiment of the invention, a medical record is provided of an individual patient comprising: diagnostic evaluation determining development or existence of a chronic disease or disorder; and theragnostic evaluation of the patient based upon therapeutic appropriateness, therapeutic guidance, and/or therapeutic effectiveness, and often also including selection of an alternative therapeutic strategy, the evaluation leading to selection of a treatment strategy. Often, the record will further provide patient identification information, patient medical history data, patient therapy adherence data, therapeutic assurance data, patient health insurance data, therapy payment data, and/or details on execution and progression of the selected treatment strategy. Additional aspects of the inventions include a database comprising a plurality of such medical records, e.g., wherein: a large majority of the medical records in the database include treatment response data; in some medical records the treatment strategy is complete and the patient has achieved remission or excellent response; the database comprises at least 2000 medical records with treatment response data; the database is in a form of electronic, optical, paper, or some combination; the database further comprises one or more of patient identification data, patient medical history data, patient health insurance data, patient therapy adherence data, therapeutic assurance data, or therapy payment data; the database comprises response data from alternative treatments of different patients; the database further incorporates a mechanism to identify when the therapeutic strategy for a patient differs from the accepted therapeutic guidelines; and/or the database further incorporates a mechanism to identify when the response of a patent to an alternative therapeutic strategy differs from the expected response to accepted therapeutic guidelines. The invention provides an entity selected from: a pharmacy; a pharmacy benefits management entity; an employer or insurance entity, including a health insurer, commercial health insurer, or public/government health insurer; a healthcare provider entity, including a clinic, hospital, outpatient facility, specialty clinic, or physician's office; a pharmaceutical company; or a diagnostic company; which uses or possesses a medical record, as described, or a database, as described.

### BRIEF DESCRIPTION OF THE DRAWINGS

The invention is best understood from the following detailed description when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures.

FIG. 1 indicates essential stakeholders of the Klaritos platform. A marketed, specialty drug can be efficiently delivered directly to the patient or her physician through Klaritos' formulary and specialty pharmacy. Klaritos platform is here depicted as a telehealth medium consisting of: drug formulary, specialty pharmacy, disease and therapy management care, and theragnostic laboratories. A disease population, e.g., rheumatoid arthritis, can be stratified into distinct subsets and appropriate specialty drugs are administered to achieve excellent therapeutic efficacy. KlariPay provides therapeutic efficacy assurance to payers and employers. Theragnostics-mediated therapeutic guidance is provided by theragnostic laboratories. The platform further provides product differentiation and presumably market enrichment for a specialty drug.

FIG. 2 provides the workflow involved in a Klaritos platform. Steps 1-5 represent the current treatment model that is generally followed by specialty physicians and payers. Steps 6-17 involve the components of Klaritos platform. See Example-1.

FIG. 3 provides a pathway of money flow when therapeutic efficacy assurance is achieved. KlariPay is the financial transaction platform between a payer(s) and an assurance company or theragnostic platform company including e.g., prescription drug plan. A pharmaceutical company and (or) a financial risk assurance (re-insurance) company may involve as stakeholders in this transaction.

FIG. 4 provides a pathway of money flow when therapeutic efficacy assurance threshold is not achieved. When a pharmaceutical company and (or) a financial risk assurance (re-insurance) company is not part of such assurance, such assurance is provided by KlariPay to payers.

### DETAILED DESCRIPTION

The present disclosure provides methods, processes, reagents and kits for novel healthcare delivery, treatment and payment, particularly for achieving improved economic and treatment outcomes in patients treated with specialty drugs. Methods, processes, and kits provide several uses, including, for providing therapeutic guidance, for providing therapeutic efficacy assurance, for providing product differentiation and presumably market enrichment for a specialty drug, for delivering the drug directly from a pharmaceutical company to a patient or to her specialty physician through a novel healthcare delivery platform.

Before the present invention is further described, it is to be understood that this invention is not limited to particular embodiments described, as such may, of course, vary. It is also to be understood that the terminology used herein is for the purposes of describing particular embodiments only, and is not necessarily intended to be limiting.

It is also to be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In addition, the use of “or” means “and/or” unless stated otherwise. Similarly, “comprise,” “comprises,” “comprising,” “include,” “includes,” and “including” are interchangeable and not intended to be limiting. Where descriptions of various embodiments use the term “comprising,” those skilled in the art would understand that in some specific instances, an embodiment can be alternatively described using language “consisting essentially of” or “consisting of.”

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Although many processes, methods and materials similar or equivalent to those described herein can also be used in the practice or testing of the present invention, the preferred processes, methods and materials are now described. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In some embodiments, methods recited herein may be carried out in any order of the recited events which is logically possible, as well as the recited order of events.

The section headings used herein are for organizational purposes only and not to be construed as limiting the subject matter described.

### Definitions

### Specialty Drugs

Specialty drugs are fairly expensive drugs prescribed by specialist doctors to treat complex, chronic, rare, or difficult-to-manage diseases and disorders such as cancers, autoimmune diseases, inflammatory disorders, chronic viral infections, etc. Examples of specialty drugs include therapeutic antibodies, protein and peptide therapies, small molecules, therapeutic vaccines, stem cell therapies, and blood derivatives including IVIG therapies. A specialty drug typically meets five or more of the following criteria: specialist-initiated (e.g., oncologist, rheumatologist); biotech product (covers both IP protected drugs, generics and biogenerics); injectable formulation; costs more than $6,000 per year; requires special handling; limited distribution; necessitates risk evaluation and mitigation strategies (REMS) program (Duffant, et al. (2014) Succeeding in the Rapidly Changing U.S. Specialty Market IMS Health).

Administration of specialty drugs is typically via self-administered injections or intravenous infusions in the physician's office, specialized administration facility, or in a hospital setting. In some instances, specialty drugs are orally administered. Not all biologic drugs come under specialty drugs (e.g., insulins). In some other instances, small molecule drugs can be categorized as specialty drugs (e.g., sofosbuvir, tofacitinib).

The Centers for Medicare and Medicaid Service (CMS) defines a specialty drug as one with a minimum monthly cost of $600. Some insurance plans also set cost thresholds, which can be up to double this amount. Private payers classify specialty drugs based on the cost, with $1,154 determined as the average minimum monthly cost.

The United States spent $2.9 trillion on health care in 2013. According to the annual spending report from the CMS Office of the Actuary, the itemized spending is on hospital care ($936.9 billion), physicians and clinical services ($586.7 billion), and prescription drugs ($271.1 billion). Overall the healthcare spending in the United States is expected to continue to rise. The CMS actuary's most recent projections predict that healthcare spending will almost double to $5.2 trillion in 2023, when it will account for 19.3 percent of the economy. Specialty drug spending is on the rise and is expected to reach $1.7 trillion in 2030, and this will account for an estimated 44% of a health plan's total drug expenditure in 2030 (Duffant, et al. (2014) Succeeding in the Rapidly Changing U.S. Specialty Market IMS Health).

Several factors drive the specialty drug spend, including: (a) the rising interest in personalized medicine and targeted therapies, e.g., stratified medicine, and therapies to treat rare and orphan disorders: approximately 700 specialty drugs are in clinical development; (b) price inflation has been a leading driver with prices of some drugs growing at double-digit rates; (c) many specialty drugs including biologics are considered breakthrough therapies with few close substitutes, and that the lack of generic (and biosimilar) competition further contributes to higher prices for these drugs; (d) specialty drugs, either distributed through specialty pharmacies or specialty distributors, are frequently administered by medical professionals at higher-cost treatment sites such as hospitals, infusion centers and physician offices. These drugs also require special handling, administration, patient education, and clinical support—all of which further drive up their cost, either directly or indirectly with associated services.

The costs of new drugs entering the market rise continuously across all cancers and diseases. Of the 12 drugs approved by the US Food and Drug Administration (FDA) for different cancer indications in 2012, 11 cost more than $100,000 per year. The prices for oncology agents have nearly doubled in the past decade, from an average of $5000 per month to more than $10,000 per month. Examples include therapies approved by the US Food and Drug Administration (FDA) in 2012 for the treatment of CML: bosutinib, ponatinib, and omacetaxine. This is in addition to 3 other drugs approved in the last decade: imatinib, dasatinib, and nilotinib. The 3 new drugs, however, have been priced at: ponatinib at $138,000 per year, omacetaxine at $28,000 for induction and $14,000 per maintenance course, and bosutinib at ˜$118,000 per year (About, et al. (2013) Blood 121:4439).

The targeted cancer therapies costs are very high. Many of them are priced between $6000 to 12,000 per month, or approximately $70,000 to $115,000, annually. Brentuximab (Adcetris, Seattle Genetics/Millennium-Takeda Oncology), which was recently approved in the United States for Hodgkin's lymphoma and systemic anaplastic large cell lymphoma, costs about $5000 per vial. Patients typically need 3 vials per dose, and usually 7 to 9 doses per course of treatment resulting in a total of $135,000 or more.

Ipilimumab (Yervoy, Bristol-Meyers Squibb) is used to treat melanoma, costs $30,000 per injection, which translates to $120,000 for a course of therapy, based on the approved dosing regimen of 3 mg/kg every 3 weeks for 4 doses.

In addition to targeted therapies, novel or reformulated chemotherapy drugs are also priced very high. These include pralatrexate (Folotyn, Allos Therapeutics), at $120,000 per course; omacetaxine (Synribo, Teva Pharmaceuticals), at $28,000 for induction and $14,000 for monthly treatments; and pegylated asparaginase (Oncaspar, Sigma-Tau Pharma.), at $22,000 (Kantarjian, et al. (2013) J. Clin. Oncol. 31:3600-3604).

Expensive drugs are also being developed and approved for other, non-oncology, medical conditions. One such agent is ivacaftor (Kalydeco, Vertex Pharmaceuticals), which is the first drug that targets the underlying molecular defect in cystic fibrosis. It is designed to treat the disease in a small subpopulation of patients who carry a specific genetic mutation, G551D, and costs $311,000 a year—making it one of the most expensive drugs currently on the market. Of the ˜30,000 U.S. patients with cystic fibrosis only ˜1200 patients carry this mutation.

Another high-priced drug is sofosbuvir (Sovaldi, Gilead), which is priced at $1000 per pill, or $84,000 for 12 weeks of treatment. The drug has been shown to be highly effective for treating hepatitis C virus, which afflicts more than 3 million people in the United States. Because sofosbuvir needs to be taken in combination with other drugs, full treatment can cost upward of $100,000, because some patients require re-treatment.

Gilead has developed Harvoni to treat hepatitis C virus. It is a ledipasvir/sofosbuvir (Harvoni) combination drug that is the first treatment that does not require administration with either interferon or ribavirin. The current price of the drug is $63,000 for 8 weeks of treatment, $94,500 for 12 weeks, and $189,000 for 24 weeks. But these costs might be lower than for sofosbuvir, because it is taken without companion medications (ribavirin, interferon) with serious side effects, and because many patients will only require 8 weeks of therapy.

Specialty drugs to treat relapsing-remitting multiple sclerosis include Peginterferon β-1a (Plegridy, Biogen), listed at $62,036 for a year's treatment; dimethyl fumarate (Tecfidera, Biogen), priced at $60,121 a year.

Specialty drugs to treat various autoimmune diseases such as rheumatoid arthritis, Crohn's disease, ulcerative colitis, psoriasis, psoriatic arthritis cost approximately $20,000 or more per year. Some of these drugs are infliximab, etanercept, adalimumab, rituximab, certolizumab pegol, golimumab, tocilizumab, abatacept, etc. Several biosimilars and biobetters are being developed for many of these drugs. Another specialty drug pill to treat rheumatoid arthritis, tofacitinib (Xeljanz, Pfizer), is priced at $24,600 a year.

Some of the approved specialty drugs (injectables, oral/topical) to treat various cancers include: Abraxane, Adcetris, Afinitor, Arranon, Arzerra, Avastin, Beleodaq, Blincyto, Bosulif, Caprels, Cometriq, Crinone, Cyramza, Dacogen, Eligard, Elspar, Erbitux, Erivedge, Farydak, Firmagon, Folotyn, Gazyva, Gilotrif, Gleevec, Halaven, Herceptin, Hycamtin capsules, Hycamtin (topotecan injection), Ibrance, Iclusig, Imburvica, Inlyta, Intron A, Istodax, Ixempra, Jakafi, Jevtana, Kadcyla, Kepivance, Keytruda, Kyprolis, Levoleucovorin Calcium, Lupron Depot, Marqibo, Matulane, Mekinist, Mugard, Nexavar, Novantrone (mitoxantrone), Oncospar, Opdivo, Perjeta, Pomalyst, Proleukin, Prothelial, Provenge, Purixan, Revlimid, Rituxan, Sprycel, Stivarga, Sutent, Sylatron, Sylvant, Synribo, Tafinlar, Tarceva, Targretin, Tasigna, Temodar, Testopel, Thalomid, Thyrogen, Torisel, Treanda, Tykerb, Valchlor, Valstar, Vantas, Vectibix, Velcade, Vidaza, Votrient, Xalkori, Xeloda, Xgeva, Xofigo, Xtandi, Yervoy, Zaltrap, Zelboraf, Zoladex, Zolinza, Zometa, Zydelig, Zykadia, Zytiga.

Some of the approved specialty drugs to treat multiple sclerosis include: Ampyra, Aubagio, Avonex, Betaseron, Copaxone, Extavia, Gilenya, Lemtrada, Mitoxantrone, Plegridy, Rebif, Tecfidera, Tysabri.

Some of the approved specialty drugs to treat inflammatory indications such as rheumatoid arthritis are: Actemra, Arcalyst, Benlysta, Cimzia, Enbrel, Entyvio, Humira, Ilaris, Kineret, Krystexxa, Orencia, Ortexup, Rasuvo, Remicade, Rituxan, Simponi, Simponi Aria, Stelara, Xeljanz.

Some of the approved specialty drugs to treat inflammatory bowel diseases such as Crohn's and ulcerative colitis are: Cimzia, Entyvio, Humira, Remicade, Simponi, Tysabri.

Some of the approved specialty drugs to treat psoriasis are Cosentyx, Enbrel, Humira, Otezla, Otrexup, Rasuvo, Remicade, Stelara.

Some of the approved specialty drugs to treat osteoarthritis include: Euflexxa, Gel-One, Hyalgan, Monovisc, Orthovisc, Supartz, Synvisc.

Some of the approved specialty drugs to treat osteoporosis include: Boniva, Forteo, Prolia, Reclast.

An approved specialty drug to treat systemic lupus erythematosus: Benlysta.

Some of the approved specialty drugs to treat ophthalmic conditions include: Cystaran, Eylea, Jetrea, Iluvien, Lucentis, Macugen, Ozurdex, Retisert, Visudyne.

Some of the approved specialty drugs to treat immune deficiency include: Actimmune, Bivigam, Carimune, Cytogam, Flebogamma, Gamastan S-D, Gammagard Liquid, Gammagard S-D, Gammaked, Gammaplex, Gamunex-C, Hizentra, Hyqvia, Octagam, Privigen.

Some of the approved specialty drugs to treat blood cell deficiency include: Aranesp, Epogen, Granix, Leukine, Mozobil, Neulasta, Neumega, Neupogen, Nplate, Proctir, Promacta.

Some of the approved specialty drugs to treat alpha-1 deficiency include: Aralast NP, Glassia, Zemaira, Prolastin C.

Some of the approved specialty drugs to treat anticoagulant include: Arixtra, Fragmin, Privask, Lovenox.

Some of the approved specialty drugs to treat enzyme deficiency and lysosomal storage disorders include: Adagen, Aldurazyme, Carbaglu, Cerdelga, Cerezyme, Cystagon, Elaprase, Elelyso, Fabrazyme, Lumizyme, Myozyme, Naglazyme, Orfadin, Sucraid, VPRIV, Vimizim, Vpriv, Zavesca.

Some of the approved specialty drugs to treat asthma and allergy include: Xolair, Oralair.

Some of the approved specialty drugs to treat growth deficiency include: Genotropin, Humatrope, Increlex, Norditropin, Nutropin AQ, Omnitrope, Saizen, Serostim, Tev-Tropin, Zorbtive.

Some of the approved specialty drugs to treat hepatitis C virus (HCV) include: Infergen, Olysio, Pegasys, Peg-Intron, Ribavirin (Rebetol, Copgeus), Ribasphere, Ribapak, Ribavirin (Moderiba); Sovaldi, Harvoni, Victrelis.

Some of the approved specialty drugs to treat human immunodeficiency virus (HIV) include: Aptivus, Atripla, Combivir (lamivudine/zidovudine), Complera, Crixivan, Edurant, Egrifta, Emtriva, Epivir, Epzicom, Fuzeon, Intelence, Invirase, Isentress, Kaletra, Lexiva, Norvir, Prezista, Rescriptor, Retrovir, Reyataz, Selzentry, Stribild, Sustiva, Tivicay, Triumeq, Trizivir, Truvada, Tybost, Videx, Videx EC, Viracept, Viramune, Viramune XR, Viread, Zerit, Ziagen.

Some of the approved specialty drugs to treat pulmonary hypertension include: Adcirca, Adempas, Flolan, Flolan Diluent, Letairis, Opsumit, Orenitram, Remodulin, Revatio, Tracleer, Tyvaso, Veletri, Ventavis.

An approved specialty drug (antibody) to treat respiratory synctial virus is: Synagis.

Some of the approved specialty drugs to treat cystic fibrosis include: Bethkis, Cayston, Kalydeco, Pulmozyme, Tobi (tobramycin), Tobi Podhaler.

Some of the approved specialty drugs in the contraceptive space include: Mirena, Nexplanon, Paragard, Skyla.

Some of the approved specialty drugs to treat infertility include: Bravelle, Cetrotide, Chorionic Gonadatropin (brands include Novarel, Pregnyl), Crinone, Endometrin, Follistim AQ, Ganirelix, Gonal-F, leuprolide, Menopur, Ovidrel, progesterone, injection, Repronex.

Some of the approved specialty drugs to treat lipid disorders (PCSK9 inhibitors) include: Praluent, Repatha.

Some of the approved specialty drugs to treat miscellaneous specialty conditions include: Acthar H. P. Gel, Apokyn (movement disorder), Arestin, Botox (botulinum toxin), Botox Cosmetic, Ceprotin (coagulation disorder), Chenodal, Cystadane, Dysport (botulinum toxin), Gattex (gastrointestinal disorders), Hetlioz, Juxtapid, Kynamro, Kuvan (phenylketonuria), Makena (pre-term birth), Myalept, Myobloc (botulinum toxin), Northera (movement disorder), Prialt, Procysbi, Qutenza, Ravicti, Sabril, Solesta (gastrointestinal disorders), Soliris (Paroxysmal nocturnal hemoglobinuria), Viitrol, Xenazine, Xeomin, Xiaflex, Xyrem.

Some of the approved specialty drugs to treat hemophilia include: Advate, Alphanate, Alphanine SD, Alprolix, Bebulin, Benefix, Corifact, DDAVP, Eloctate, FeibaNF, Helixate FS, Hemofil M, Humate-P, Koate-DVI, Kogenate FS, Monoclate-P, Mononine, Novoseven RT, Profilnine SD, Recombinate, RiaSTAP, Rixubis, Stimate, Tretten, Wilate, Xyntha.

Some of the approved specialty drugs to treat endocrine disorders include: Aveed, Korlym, Kuvan, Lupaneta Pack, Lupron Depot-Ped, Ruconest, Sandostatin, Sandostatin LAR, Signifor, Somatuline Depot, Somavert, Supprelin LA.

### Specialty Drugs: Rare Diseases and Orphan Diseases

“Rare disease” refers to a disease or disorder affecting fewer than 1 in 2000 in Europe. A disease or disorder is defined as rare in the USA when it affects fewer than 200,000 Americans at any given time. One rare disease may affect only a handful of patients in the EU (European Union), and another may affect as many as 245,000. In the EU, as many as 30 million people alone may be affected by one of over 6,000 rare diseases existing (www.rarediseases.org). These disorders are characterized by a broad diversity of disorders and symptoms that vary not only from disease to disease but also from patient to patient suffering from the same disease. Though these phrases are used interchangeably, an orphan disease need not be a rare disease. Generally, orphan diseases also include neglected diseases which inflict severe health burdens on the world's poorest people. Examples include lymphatic filariasis, malaria, leishmaniasis, etc.

“Ultra-rare disease” refers to a disease affecting fewer than 20 patients per million of population (or, one patient per 50,000 people). Most ultra-rare diseases affect far fewer than this, as few as one per million or less.

Almost invariably all drugs developed to treat rare and ultra-rare diseases are specialty drugs. The therapy cost for these drugs can be $100,000 or more, and some $300,000-500,000 per patient per year. These drugs are typically priced very high because the number of treatable patients are generally very low in developed countries.

The cost of Aldurazyme (Laronidase; enzyme replacement therapy; Genzyme) for mucopolysaccharidosis-I can range from $200,000 in children to $500,000 in adults. Vimizin (elosulfase alpha; BioMarin) costs $380,000 per year to treat Morquio A syndrome. Alexion Pharmaceuticals' Soliris (eculizumab) is a $440,000-a-year treatment for paroxysmal nocturnal hemoglobinuria (PNH) and atypical hemolytic uremic syndrome (aHUS), while Cinryze (C1 esterase inhibitor [human]; Viropharma) treats hereditary angioedema for $417,000 a year.

Some of the FDA-approved BLA-designated drugs to treat rare diseases include: elosulfase alfa (Vimizim; mucopolysaccharidosis type IVA, Morquio A syndrome); Metreleptin (Myalept; leptin deficiency with congenital or acquired generalized lipodsytrophy); ramucirumab (Cyramza; advanced gastric cancer or gastro-esophageal adenocarcinoma); siltuximab (Sylvant; multicentric Castleman's disease); pembrolizumab (Keytruda; unresectable or metastatic melanoma and disease progression following ipilimumab and, if BRAF V600 mutation positive, a BRAF inhibitor); blinatumomab (Blincyto; Philadelphia chromosome-negative relapsed or refractory B-cell precursor acute lymphoblastic leukemia); nivolumab (Opdivo; unresectable or metastatic melanoma and disease progression following ipilimumab and, if BRAF V600 mutation positive, a BRAF inhibitor).

Some of the FDA-approved, orphan designated supplement approvals include: trametinib (Mekinist; unresectable or metastatic melanoma with BRAF V600E or V600K mutations, as detected by FDA-approved test); dabrafenib (Tafinlar; unresectable or metastatic melanoma with BRAF V600E or V600K mutations, as detected by FDA-approved test); PCI-32765 (Ibrutinib; mantle cell lymphoma who have received at least one prior therapy; chronic lymphocytic leukemia who have received at least one prior therapy); escallantide (Kalbitor; acute attacks of hereditary angioedema in patients 12 years of age or older); ethiodized oil (Lipiodol; hysterosalpingography in adults, lymphography in adult and pediatric patients, selective hepatic intra-arterial use for imaging tumors in adults with known hepatocellular carcinoma); ofatumumab (Arzerra; in combination with chloranmbucil for the treatment of previously untreated patients with CLL for whom fludarabine-based therapy is considered inappropriate; CLL refractory patients to fludarabine and alemtuzumab); Lymphoseek; Zydelig (relapsed CLL, in combination with rituximab, in patients for whom rituximab alone would be considered appropriate therapy due to other co-morbidities; relapsed B-cell non-Hodgkin's follicular lymphoma (FL) in patients who have received at least two prior systemic therapies; relapsed small lymphocytic lymphoma (SLL) in patients who have received at least two prior systemic therapies); PCI-32765 (Ibrutinib; patients with: mantle cell lymphoma (MCL) who have received at least one prior therapy, chronic lymphocytic leukemia (CLL) who have received at least one prior therapy or chronic lymphocytic leukemia with 17p deletion); alglucosidase alpha2 (Lumizyme; Pompe disease, acid a-glucosidase GAA deficiency); bortezomib (Velcade; Treatment of patients with multiple myeloma and patients with mantle cell lymphoma who have received at least 1 prior therapy); eltrombopag (Promacta; Thrombocytopenia in patients with chronic immune (idiopathic) thrombocytopenia (ITP) who have had an insufficient response to corticosteroids, immunoglobulins, or splenectomy. Thrombocytopenia in patients with chronic hepatitis C to allow the initiation and maintenance of interferon-based therapy. Patients with severe aplastic anemia who have had an insufficient response to immunosuppressive therapy); talglucerase alfa (Elelyso; Long term enzyme replacement therapy (ERT) for adult and pediatric patients with a confirmed diagnosis of Type 1 Gaucher disease); adalimumab (Humira; expanded indication: Reducing signs and symptoms and inducing and maintaining clinical remission in patients 6 years of age and older with moderately to severely active Crohn's disease who have had an inadequate response to corticosteroids or immunomodulators such as azathioprine, 6-mercaptopurine or methotrexate); adalimumab (Humira; expanded indication: Reducing signs and symptoms of moderately to severely active polyarticular Juvenile Idiopathic Arthritis (JIA) in patients 2 years of age and older); bortezomib (Velcade; multiple myeloma or mantel cell lymphoma); ramucirumab (Cyramza; advanced gastric or gastro-esophageal junction adenocarcinoma, as a single agent or in combination with paclitaxel); bevacizumab (Avastin; cervical cancer, in persistent, recurrent, or metastatic disease; platinum-resistant recurrent epithelial ovarian, fallopian tube or primary peritoneal cancer); cinacalcet HCl (Sensipar; hypercalcemia in adult patients with parathyroid carcinoma); ruxoutinib phosphate (Jakafi; intermediate or high-risk myelofibrosis, including primary myelofibrosis); denosumab (Xgeva; skeletal-related events in patients with bone metastases from solid tumors; giant cell tumor of bone; hypercalcemia of malignancy refractory to bisphosphonate therapy); aripiprazole (Abilify; Tourette's, severe autism; agitation associated with schizophrenia or bipolar mania); lanreotide (Somatuline Depot; unresectable, well/moderately differentiated, locally advanced or metastatic gastroenteropancreatic neuroendocrine tumors).

FDA-approved drugs to treat ultra-rare diseases include: Procysbi. (nephropathic cystinosis); Soliris (atypical hemolytic uremic syndrome); Soliris (paroxysmal nocturnal hemoglobinuria).

Because of the extraordinary cost of these therapies, only a small fraction of the eligible patients can afford these therapies in the United States or Europe.

### Targeted Therapies

Several targeted therapies are approved by the FDA. Nearly all of these drugs are specialty drugs. The approval or administration of these therapies is guided by companion diagnostic products which are also approved by FDA.

Pembrolizumab (Keytruda; Merck) is approved for treating NSCLC cancer. PD-L1 IHC 22C3 pharmDx is a qualitative immunohistochemical assay for use in the detection of PD-L1 protein in formalin-fixed, paraffin-embedded (FFPE) non-small cell lung cancer (NSCLC) tissues. PD-L1 protein expression is determined by using Tumor Proportion Score (TPS), which is the percentage of viable tumor cells showing partial or complete membrane staining. The specimen should be considered PD-L1 positive if TPS≥50% of the viable tumor cells exhibit membrane staining at any intensity.

The Therascreen® EGFR RGQ PCR Kit is a real-time PCR test for the qualitative detection of exon 19 deletions and exon 21 (L858R) substitution mutations of the epidermal growth factor receptor (EGFR) gene in DNA derived from formalin-fixed paraffin-embedded (FFPE) non-small cell lung cancer (NSCLC) tumor tissue. The test is intended to be used to select patients with NSCLC for whom GILOTRIF® (afatinib) or IRESSA® (gefitinib), EGFR tyrosine kinase inhibitors (TKIs), is indicated. Safety and efficacy of GILOTRIF (afatinib) and IRESSA (gefitinib) have not been established in the patients whose tumors have L861Q, G719X, S768I, exon 20 insertions, and T790M mutations, which are also detected by the therascreen EGFR RGQ PCR Kit.

The Cobas® KRAS Mutation Test, for use with the Cobas® 4800 System, is a real-time PCR test for the detection of seven somatic mutations in codons 12 and 13 of the KRAS gene in DNA derived from formalin-fixed paraffin-embedded human colorectal cancer (CRC) tumor tissue. The test is intended to be used as an aid in the identification of CRC patients for whom treatment with Erbitux® (cetuximab; IgG₁) or with Vectibix® (panitumumab; IgG₂) may be indicated if mutations are not detected. A second companion diagnostic product, therascreen KRAS RGQ PCR Kit (Qiagen Manchester, Ltd.) is also available to aid in the identification of CRC patients for treatment with Erbitux (cetuximab) and Vectibix (panitumumab) based on a KRAS if no mutation were detected. Another CDx product DAKO EGFR PharmDx kit (Dako North America, Inc.) is approved, indicated as an aid in identifying CRC patients eligible for treatment with Erbitux (cetuximab) or Vectibix (panitumumab). The EGFR pharmDx™ assay is a qualitative immunohistochemical (IHC) kit system to identify epidermal growth factor receptor (EGFR) expression in normal and neoplastic tissues routinely-fixed for histological evaluation.

BRACAnalysis CDx™, developed by Myriad Genetic Laboratories, is an in vitro diagnostic device intended for the qualitative detection and classification of variants in the protein coding regions and intron/exon boundaries of the BRCA1 and BRCA2 genes using genomic DNA obtained from whole blood specimens collected in EDTA. Single nucleotide variants and small insertions and deletions (indels) are identified by polymerase chain reaction (PCR) and Sanger sequencing. Large deletions and duplications in BRCA1 and BRCA2 are detected using multiplex PCR. Results of the test are used as an aid in identifying ovarian cancer patients with deleterious or suspected deleterious germline BRCA variants eligible for treatment with Lynparza™ (olaparib).

The FerriScan R2-MRI Analysis System (Resonance Health Analysis Services Pty Ltd) is intended to measure liver iron concentration to aid in the identification and monitoring of non-transfusion dependent thalassemia patients receiving therapy with deferasirox (Exjade; Novartis).

The c-Kit pharmDX assay (Dako North America, Inc.) is an IHC kit system for the differential diagnosis of gastrointestinal stromal tumors (GIST). After diagnosis of gastrointestinal stromal tumor (GIST), results from c-Kit pharmDx may be used as an aid in identifying those patients eligible for treatment with imatinib mesylate (Gleevec/Glivec; Novartis).

The Inform Her-2/Neu gene detection system (Ventana Medical Systems, Inc.) is a fluorescence in situ hybridization (FISH) DNA probe assay that determines the qualitative presence of Her-2/Neu gene amplification on formalin-fixed, paraffin embedded human breast tissue as an aid to stratify breast cancer patients according to risk for recurrence or disease-related death. The test is used to select the patients eligible for trastuzumab (Herceptin; Genentech/Roche) treatment.

The PathVysion HER-2 DNA Probe Kit (P980024 S001-S012; PathVysion Kit; Abbott Molecular, Inc.), PATHWAY ANTI-HER-2/NEU (P990081 S001-S028; Ventana Medical System, Inc.), INSITE HER-2/NEU KIT (P040030; Biogenex Laboratories, Inc.) are designed to detect amplification of the HER-2/neu at the gene or protein expression level. Additionally approved tests are: SPOT-LIGHT HER2 CISH KIT (050040 S001-S003; Life Technologies, Inc.), Bond Oracle Her2 IHC System (P090015-S001; Leica Biosystems), HER2 CISH PharmDx Kit (P100024 S001-S005; Dako Denmark A/S); INFORM HER2 DUAL ISH DNA Probe Cocktail (P100027 S001-S017; Ventana Medical Systems). These kits are used as an aid in the assessment of patients for whom trastuzumab (Herceptin; Genentech/Roche) treatment is being considered.

HercepTest (P980018 S001-S018; Dako Denmark A/S) is a semi-quantitative immunocytochemical assay to determine HER2 protein overexpression in breast cancer tissues routinely processed for histological evaluation and formalin-fixed, paraffin-embedded cancer tissue from patients with metastatic gastric or gastroesophageal junction adenocarcinoma. HercepTest is indicated as an aid in the assessment of breast and gastric cancer patients for whom Herceptin (trastuzumab) treatment is being considered; and for breast cancer patients for whom PERJETA (pertuzumab; Genentech/Roche) treatment or KADCYLA (ado-trastuzumab emtansine; Genentech/Roche) treatment is being considered.

The THxID BRAF kit (P120014; bioMérieux Inc.) is an in vitro diagnostic device intended for the qualitative detection of the BRAF V600E and V600K mutations in DNA samples extracted from formalin-fixed paraffin-embedded (FFPE) human melanoma tissue. It is an aid in selecting melanoma patients whose tumors carry the BRAF V600E mutation for treatment with dabrafenib [Tafinlar; Novartis] and as an aid in selecting melanoma patients whose tumors carry the BRAF V600E or V600K mutation for treatment with trametinib [Mekinist; Novartis].

The Cobas® EGFR Mutation Test (P120019 S001-S004; Roche Molecular) is a real-time PCR test for the qualitative detection of exon 19 deletions and exon 21 (L858R) substitution mutations of the epidermal growth factor receptor (EGFR) gene in DNA derived from formalin-fixed paraffin-embedded (FFPET) human non-small cell lung cancer (NSCLC) tumor tissue. The test is intended to be used as an aid in selecting patients with NSCLC for whom erlotinib (Tarceva®; Genentech/Roche) is indicated.

VENTANA ALK (D5F3) CDx Assay (P140025; Ventana Medical Systems, Inc.) is intended for the qualitative detection of the anaplastic lymphoma kinase (ALK) protein in formalin-fixed, paraffin-embedded (FFPE) non-small cell lung carcinoma (NSCLC) tissue stained with a BenchMark XT automated staining instrument. It is indicated as an aid in identifying patients eligible for treatment with crizotinib (XALKORI®; Pfizer).

The Vysis ALK Break Apart FISH Probe Kit (P110012 S001-S003; Abbott Molecular Inc.) is a qualitative test to detect rearrangements involving the ALK gene via fluorescence in situ hybridization (FISH) in formalin-fixed, paraffin-embedded (FFPE) non-small cell lung cancer (NSCLC) tissue specimens to aid in identifying patients eligible for treatment with crizotinib (Xalkori®; Pfizer). This is for prescription use only.

The Cobas 4800 BRAF V600 Mutation Test (P110020 S001-S010; Roche Molecular Systems) is an in vitro diagnostic device intended for the qualitative detection of the BRAF V600E mutation in DNA extracted from formalin-fixed, paraffin-embedded human melanoma tissue. The Cobas 4800 BRAF V600 Mutation Test is a real-time PCR test on the Cobas 4800 system, and is intended to be used as an aid in selecting melanoma patients whose tumors carry the BRAF V600E mutation for treatment with vemurafenib (Zelboraf®; Genentech/Roche).

### Treatment Costs of Specialty Drugs

Yet another reason for the increasing cost of specialty drugs, and, e.g., cancer drugs, has nothing to do with the prices set by the pharmaceutical industry but it is related to how the oncology medicine practice is evolving in the United States. Cancer drug costs can vary depending on who is administering the drug. If a chemotherapy is administered in a hospital outpatient setting instead of a physician's office, costs can be as much as 53% higher.

Even if the treatment is still delivered in the physician office, once the practice has been purchased by a hospital system and is no longer independent, prices go up. One reported example is for a breast cancer patient who had been receiving trastuzumab therapy (Herceptin, Roche/Genentech). The initial charge was approximately $5,100 per month for the drug; subsequent to acquisition of this oncology practice by a hospital system, it was priced $16,000, even though it was being delivered by the same oncology practice in the same office.

Another reason for high drug costs is that physicians often have a monetary incentive to use a more expensive drug, especially where payers do not have mechanisms in place to minimize expenditures. After the passage of the Medicare Modernization Act of 2003, reimbursement was set at the average sales price plus a 6% mark-up to cover practice costs. In some cases, such as with carboplatin, this 6% mark-up does not even cover the cost of administering the drug. The drug has fallen in price from $125.00 to $3.50, which makes the 6% payment exceedingly low. To make up for this, some oncologists have switched to using higher-margin brand-name drugs. Instead of using generic paclitaxel and earning 6% of $312, e.g., they use Abraxane, a branded protein-bound version of paclitaxel, and earn 6% of $5824. This so-called “buy and bill” practice can create a very substantial incentive to use more expensive drugs. Thus, a $6 mark-up on a $100 treatment is very low, but a $6000 mark-up on a $100,000 treatment amounts to conflict of interest.

### Specialty Drugs—Supply Chain Distribution and Channels

Several key stakeholders are involved in the supply, delivery, and treatment chain of specialty drugs. These stakeholders may include payers, employers including pension funds, physicians, and patients. In addition, pharmacy benefit management companies (PBM), specialty pharmacies, specialty distributors and providers (hospitals) are often involved. See Duffant, et al. (2014) Overview of the Specialty Drug Trend: Succeeding in the Rapidly Changing U.S. Specialty Market IMS Health white paper, and references cited therein.

“Payer” in healthcare generally refers to entities that finance or reimburse the cost of drugs, devices, and related healthcare services. In most cases, this term refers to insurance carriers, other third-party payers, or health plan sponsors (e.g., employers, unions, pension funds). If a patient pays for any of the products and services, albeit a portion of this payment, e.g., 5%, 15%, or more, for payment purposes he is considered as a payer, and this amount is referred to as coinsurance amount.

“Pharmacy Benefit Management” refers to a third-party administrator of prescription drug programs for plan sponsors (e.g., employers and health plans). PBMs are generally responsible for developing and maintaining the drug formulary, e.g., a listing of approved and available drugs in the prescription drug plan and formulary; managing utilization and cost; contracting with pharmacies; negotiating discounts and rebates with drug manufacturers; and processing and paying prescription drug claims. Some PBMs also offer value-added services such as patient therapeutic adherence and compliance and therapy management programs that help high risk patients stay on their medications and avoid drug-related complications. PBMs manage pharmacy benefits. In the drug delivery supply chain continuum, PBMs may be an extension of a payer and may provide specialty pharmacy services. Currently, however, features such as therapeutic efficacy assurance including the associated financial assurance, theragnostic guidance in a patient or subset(s) of patients, disease and therapy management care, and product differentiation of specialty drugs are not provided by the current PBMs, either individually or collectively. See generally Danzon www.dol.gov/ebsa/pdf/ACDanzon061914.pdf.

“Specialty Pharmacy” refers to a pharmacy that delivers specialty drugs, typically to patients, physicians, or hospitals. Specialty pharmacies combine medication dispensing with clinical disease management. Their services have been used to improve patient outcomes and contain costs of specialty pharmaceuticals. These pharmacies may be part of independent pharmacy businesses, retail pharmacy chains, wholesalers, pharmacy benefit managers (PBMs), or health insurance companies. Presumably, benefits from more restricted specialty networks include more cost-effective pricing and less variability in patient care and experience. Specialty pharmacies manage the complex reimbursement process, with the goal of making it easier for patients, providers, and payers. PBMs can reject filling or covering a specialty pharmaceutical product if it is not dispensed through its preferred specialty pharmacy providers (SPP). These entities provide cost-management services for payers (and PBMs), including contracting with pharmaceutical manufacturers for discounted pricing, and assisting patients to obtain prior authorizations. Payer organizations can receive medication rebates directly through contracting with specific specialty vendors or through PBMs. These rebates create cost savings and are typically available for specialty pharmaceutical classes with higher utilization, such as those agents for rheumatoid arthritis and MS as well as growth hormones. Additional clinical services of specialty pharmacies include: educating patients and their caregivers about drug administration and handling; monitoring for potential adverse effects, drug interactions, and patient (therapeutic) adherence. Currently, however, features such as therapeutic efficacy assurance, theragnostic guidance in a patient or subset(s) of patients, disease and therapy management care, and product differentiation of specialty drugs are not provided by specialty pharmacies, either individually or collectively.

Specialty pharmacies are reimbursed for the drugs. Current delivery models adopt either white or brown bagging delivery. Specialty distributors and physicians are not part of the drug acquisition in these models. In some instances, a PBM may deliver or send a specialty drug directly to the patient. In this scenario, the PBM will be reimbursed for the specialty drug.

With “white bag” delivery model, insurance companies (payers) contact patients through their PBMs or in-network pharmacies and provide an option as to where they would like to have their drug sent for administration—directly to them or to a physician's office or hospital. Because many of the drugs identified for white bagging are infusion therapies, such as chemotherapy drugs, IVIG therapies, and antibody therapies, this model ensures better product integrity (e.g., proper storage, handling, package integrity, and associated labeling) than the brown bag model. Specialty drugs can be sent directly from a licensed pharmacy, e.g., a specialty pharmacy, to a licensed clinician (physician or hospital pharmacist), shipped at the correct temperature and tracked during shipping. If required, such documentation may be sent with the drug. Administration can be directly documented by a health care professional, ensuring correct dose and timing, with recordation of delivery details.

In the “brown bag” model, a specialty drug is delivered directly to patients at their homes or to be picked up at an in-network pharmacy; however, the drug is administered at physician's office or hospital. Most hospitals have a policy not to accept medications that come through the brown bag distribution model because of several issues, including a lack of any mechanism to track supply chain integrity and pedigree of the drug.

“Prior authorization” refers to a process used by some health insurance companies in the United States to determine if they will cover payment for a prescribed procedure, service, or medication. The process is intended to act as safety and cost savings measures. All, or at least most, of the specialty drugs require prior authorization. Specialty pharmacies assist patients to obtain prior authorization. Current prior authorization procedures aim at (a) drug utilization management to control cost, and (b) hopefully achieving better patient outcomes compared to all-corners strategy. However, these procedures do not aim for achieving excellent treatment responses, e.g., remission or cure in a patient or subset(s) of patients, nor do they provide efficacy or financial assurance.

“Assurance-based prior authorization” refers to the prior authorization process that is based on assurance, e.g., efficacy and financial. Such assurance is dependent on theragnostic evaluation. Efficacy assurance aims for achieving excellent treatment responses, e.g., remission or cure in a patient or subset(s) of patients.

More advanced systems, e.g., Klaritos, may combine these two concepts and further enhance the interaction with other ‘Tele’ functions to provide more extensive temporal coverage beyond a single time zone 8-hour business day, e.g., a 24/7 continuous engagement, with patients in support of their drug therapy and disease management. This may be accomplished, e.g., via a cloud based platform that supports secure, e.g., Health Insurance Portability and Accountability Act (HIPAA) compliant, databases and secure rich media communications. Advanced platforms, may support tele-consults in real time between the Klaritos team including specialist doctors and nurses, patient, patient's physician, rheumatologist or other (medical or other) specialist, and payers, as necessary, for consideration of options and selection of a drug therapy as well as for prior authorization. Such platforms might enable patient education and promote patient therapeutic adherence and compliance via video presentations, video support group participation, and video chat options, and real-time recording support for patients to self-administer medicine effectively with certain levels of certification of timing and location. Such platform also supports secure messaging (individuals and groups) to allow patients to have 24/7 access to the disease and therapy management and/or monitoring teams for both advice and addressing questions. The platform may provide support for a drug formulary and specialty pharmacy that acquires and delivers appropriate drug(s) for treatment based on therapeutic guidance (theragnostics). Such a platform can handle acquisition, and delivery of the medicines as well as handling of payment via efficient electronic financial processing systems. The platform may also be designed to support an electronic payment system for efficient and timely management of money transfers between payers, employers, patients, and pharmaceutical companies. The system may be made available via a Mobile device App and enables patients to pay their copays (co-insurance). Such payment model may facilitate automatic monitoring and determination of patient therapeutic adherence and implementation of therapeutic efficacy assurance without having to go to another agency or system. This allows timely determination of patients' remission and excellence of response as well as transfers of refunds where appropriate. Finally, patient communication, drug therapy, lab tests and compliance data can be immediately captured and integrated into an easily checked data analytics system to provide analytics on individual patients for the monitoring team for helping in therapeutic guidance.

The invention may utilize a platform (hosted, e.g., by a cloud based system) that supports drug therapy and disease management for patients. The platform may provide a combination of Telemedicine/Telepharmacy services like remote video consults, patient assessment as well as an online place for patients to monitor and help with their drug therapy. Preferably, all communication streams (audio, video and data) are encrypted, and all data and APPs are secured (encrypted and permission-accessible) to comply with HIPAA.

The platform has several novel features:
1. Live consults with DTM team and authorized team members from providers and payers allow sharing of patient's health information as well as any data imported in by providers or payers, which greatly facilitates prior authorization and/or discussions on, including changes to, treatment protocols.
2. Secure messaging for patient and others in the patient care team for asynchronous sharing of information, e.g., patient education, reminders, assessment, etc.
3. Live monitoring and guidance on medication administration and compliance. The APP reminds patients when to next administer medication; and verifies the patient medication tag on the medication container or dispenser, and automatically captures in real time video and audio of patient while self-administering medication. It may automatically update and store all this information for verification of patient therapeutic adherence and updates patient scores in therapeutic efficacy assurance model.
Patients can engage in electronic payment of coinsurance, e.g., KlariPay, as well as receiving any refunds.

### Telesystems and Patient Therapeutic Adherence, Drug Delivery and Drug Administration

Drug therapy (patient therapeutic) adherence is very important in achieving remission or excellence in treatment particularly for enabling therapeutic efficacy assurance. The methods herein provide (a) materials and tools to encourage patient therapeutic adherence, and (b) protocols to follow and achieve high compliance. The system may include, e.g., a specialty pharmacy that receives drugs from distributors or pharmaceutical companies and incorporates custom tags that link patients' medication uniquely with patient ID, dosage sequence number, treatment ID, and Drug ID, using a Computer generated QR code, e.g., a Patient Med Tag (PMT). The protocol may require the patient to scan the tag (a) when they receive the drug and (b) when they self-administer the medication, e.g., by using the App to visually scan the tag. Additional features incorporated into the protocols may include integrity checks of storage conditions, conditions of containers, download of data from package monitoring sensors (e.g., temperature extremes), and others. The App may then upload all the information from the PMT along with time and location to contemporaneously record a patient taking medication, and enables patient to visually or textually acknowledge that they have taken the medication (e.g., time and location-dependent authentications). The patient can also extend the visual connection to a conversation with the monitoring team if the patient has further questions.

This system can be designed to provide and integrate the delivery, administration of medication, collection of data, and patient therapeutic adherence assessment into a single loop such that all the steps are done in a timely and efficient manner. The system may include feedback from the patient to monitor or evaluate response to dosing, e.g., track minor issues, note indications of possible adverse reactions, etc.

### Telesystems and Therapeutic Efficacy Assurance

Telesystems may provide an e-payment or accounting system to enable fast, electronic tracking and/or payment for patients (via the APP), payers, pharmaceutical company and an efficacy assurance company. The assurance company may facilitate collection of copay (coinsurance) data from patients, and payment for acquisition of drugs. The company may manage therapeutic efficacy assurance as its own fund. In which case, it monitors closely the individual patient's suggested therapy, lab results to provide therapeutic guidance, lab results indicating efficacy, and patient therapeutic adherence for each patient. Based on these data, the company runs proprietary algorithms to determine as to how well patients have responded to the prescribed therapies and which patients are eligible for therapeutic efficacy assurance including financial assurance. No other third parties need be involved in these decision-making processes.

If patient fails the first treatment, the next best alternative treatment is proposed by DTM.

“Telemedicine” refers to the practice of improving a patient's health by permitting two-way, real-time (or near) interactive communication between a patient and a healthcare provider who are geographically separated (CMS definition). This communication is conducted via interactive telecommunications equipment that includes, at a minimum, audio and, typically, video equipment, to meet standards for telehealth set, e.g., by the U.S. Department of Health and Human Services. Other forms of less elaborate remote communication may be useful, e.g., between healthcare providers, or between healthcare assistants and a patient.

“Telepharmacy” refers to the “Practice of Telepharmacy” as “the provision of Pharmacist Care by registered Pharmacies and Pharmacists located within U.S. jurisdictions through the use of telecommunications or other technologies to patients or their agents at distances that are located within U.S. jurisdictions” (Model Act; The Model State Pharmacy Act and Model Rules of the National Association of Boards of Pharmacy). The American Society of Health-System Pharmacists (ASHP) defines telepharmacy as a method used in pharmacy practice in which a pharmacist utilizes telecommunication technology to oversee pharmacy operations or provide patient care services. Telepharmacy operations and services may include, but are not limited to: drug review and monitoring, dispensing, oral and sterile compounding verification, medication therapy management (MTM), patient assessment, and patient counseling. Analogous systems may incorporate communications outside the U.S., or similar systems in other countries may provide similar operations, including specific functions or expertise provided from disperse locations.

“Medication Therapy Management” (MTM) refers to services for individuals with multiple chronic diseases who are taking multiple medications. According to CMS guidance documents for 2013, reimbursable MTM services provided by Medicare Part D sponsors must meet the following conditions for beneficiaries: (i) a minimum of two or three chronic disease states, (ii) taking a minimum of two to eight medications, and (iii) likely to incur approx. $3,144 in annual costs for Part D drugs. Analogous management may be provided, e.g., in other countries or jurisdictions, which might approach but not technically comply with all of the above criteria.

“Disease and Therapy Management” (DTM), or “DTM Care,” herein refers to providing either or both disease management care and therapy management care. Pathophysiology of a disease and disease severity vary over time in a patient. Some subsets of patients will have extremely severe disease course in a short period versus other subsets. Similarly, some subsets of patients respond well to a therapy while others not. Thus, a patient has to be under continued medical evaluation, which is provided by DTM care. DTM care provides continued theragnostic guidance as part of the integrated delivery and treatment model, and the offerings will often include: personalized drug dosing, dosing schedule, monitoring of disease remission and relapse patterns, patient therapeutic adherence and compliance, etc. DTM care is typically managed by, or at least advised by, in-house specialist physicians and specialist nurses specialized in specific disease indications such as rheumatology, neurology, and oncology.

Patients are the ultimate end users of the drugs, diagnostic services, theragnostic products, and services. In some countries including the United States, patients also pay for some or all of the drugs and services. This form of payment is referred to as co-pay or co-insurance. From the payment context, patients are treated as one of the payers. In appropriate circumstances, guardians or agents of the patient are included, e.g., when the guardian or agent is a medical decision maker for the patient, who often may be a dependent. This is particularly true in the context of financial obligations, where the financially responsible party (e.g., guardian, agent) may be different from the patient herself.

### Pricing of Specialty Drugs

Disparity exists in drug pricing depending on any of the specific stakeholders involved in this supply chain. The payer system is heavily fragmented, and thus, contrary to popular belief, market forces do not effectively bring down the prices of specialty drugs. Price negotiations take place on an individual level in the United States, with each private insurance company negotiating with each drug company for the price of each product. Pharmacy benefits managers (PBMs), a third-party administrator of prescription drug programs primarily responsible for processing and paying prescription drug claims, will also take part in developing and maintaining the formulary, contracting with pharmacies, and negotiating discounts and rebates with drug manufacturers. Dozens of plans are available in every state, and insurance costs and plans can vary significantly from state to state or by jurisdiction. They charge different premiums and copayments, and formularies may favor different drugs purely based on contractual and pricing reasons, which leads to significant variations in pricing and out-of-pocket costs to patients.

The Centers for Medicare and Medicaid Services (CMS) is the single largest payer for healthcare in the United States, covering nearly 90 million Americans through Medicare, Medicaid, and the State Children's Health Insurance Program. Medicare itself covers approximately 50 million beneficiaries. However, by law, the federal government cannot negotiate for Medicare drug prices or obtain any sort of volume discounts. The 2003 Medicare Modernization Act explicitly prohibits the federal government from negotiating drug prices or establishing a list of preferred drugs. Currently, Part D drug prices are determined through a negotiation between the private drug plan that administers the benefit and the drug manufacturer.

However, Medicaid, the program for low-income people that is administered by the CMS, as well as the Department of Veterans Affairs (VA) are able to negotiate with drug companies for lower prices. In fact, under federal law, drug makers must provide a discount or rebate equal to at least 15% of the average manufacturer price for most brand-name drugs covered by Medicaid. Federal law also guarantees discounts for the Department of Veterans Affairs (VA), which can negotiate with drug makers to secure discounts on top of those guaranteed by law. Generally, VA is able to negotiate prices that are 25% to 50% lower than Medicare. Therefore, there exists a huge variation in drug pricing, both domestically and worldwide, and such pricing is arbitrary. Currently there are no efficient drug pricing mechanisms for specialty drugs.

### Affordability of Specialty Drugs

Payers, employers, pension funds, and patients are key players of this affordability equation in the United States. While the pharmaceutical industry contends that insurance copays (coinsurance) are too high for patients to afford, the payers argue that the drugs are too expensive and that's why patients are paying more.

Private health insurance has been under intense pressure lately. When it comes to expensive drugs, insurers are shouldering a significant share of the expense. Insurance plans generally have some type of cost-sharing program in place, depending on formularies and drug tiers. Most benefit designs have 3-tier plans, e.g., with the highest tier requiring the largest cost-sharing. Tier 1 is for generic drugs and has the lowest copayment. But plans with 4 or more tiers are becoming increasingly common, and products on the top tier (e.g., Tier 4) tend to be specialty drugs, with the highest copayment or coinsurance amounts.

One provision of the Affordable Care Act (ACA) is a maximum limit on out-of-pocket spending and cost-sharing reductions, although there are a variety of coverage options. Some policies offer lower deductibles and cost-sharing, but the tradeoff is higher monthly premiums. As an example, an individual who is enrolled in a standard (e.g., silver) plan would be responsible for no more than 6.8% of the total cost of a drug. For a drug costing $150,000 per year, that would be $10,200, but the health plan would be paying for more than 93% of the remaining cost. Still, that can be a significant amount for many patients.

Depending on the individual insurance plan, Americans generally have to pay a portion of the cost called coinsurance amount. For the 29 cancer drugs examined in the comparison study, Medicare beneficiaries paid 20% coinsurance for physician-administered drugs and a median rate of 33% coinsurance for self-administered drugs. See Cohen, et al. (2013) Health Aff. 32:762-770. For instance, these out-of-pocket costs translated into thousands of dollars for specialty drugs. For a drug costing $100,000 per year, the out-of-pocket cost for the patient can translate to $20,000-33,000 per year. For a great majority of the patients, such a drug cost structure is simply not affordable. In Europe, e.g., Germany and the United Kingdom have minimal cost-sharing whereas France and the Netherlands have no cost-sharing at all.

Because of rising costs of specialty drugs, employers are beginning to pass along a larger share of insurance costs. Large employers estimate that their health-benefit costs will rise by an average of 6.5% in 2015 (2014 annual report by National Business Group on Health). Employers cited high-cost patients, specific diseases (e.g., chronic autoimmune diseases such as multiple sclerosis, rheumatoid arthritis), and an uptick in spending for specialty drugs as the main drivers of rising costs.

In regards to the issue of access to treatment, in oncology and many chronic indications such as rheumatoid arthritis, patients go through complicated processes of treatment selection procedures. For instance, patients often go through less expensive (on the short term), step therapy or fail-this-one-first approach before they can access the medicine that will work the best for that patient.

Patient co-pays and co-insurance procedures are often quite complex, and hidden in drug formulary design. For example, Pharmacy Benefit Managers (PBMs) provide pharmacy management services for employers and their employees, and Medicare recipients through Medicare Part D Prescription Drug Plans (PDP). PBM services include prior authorization, pharmacy claims processing, dispensing prescriptions via mail order, reimbursing retail and specialty pharmacies in their network and drug formulary design and management. Employers may contract with PBMs directly or allow the health plans they use to provide medical benefits to select the PBM. PBMs charge employers for the cost of reimbursed drugs, claims adjudication and other administrative fees. Employers receive a share of pharmaceutical company rebates that the PBMs negotiate with pharmaceutical companies on branded pharmaceutical products. The PBMs develop drug formularies, with input and ultimate approval from the employer, to manage utilization and control costs. They use various tools such as formulary tiers with restricted access, prior authorization on expensive medications, step therapy where a generic must be used prior to a branded drug, and escalating patient co-pays or coinsurance depending on the cost of the medication and the formulary tier. In the end, patient co-pays may range from $0/Rx for certain generics to over $100/Rx for branded drugs dispensed at retail pharmacies. Payments for specialty drugs often require coinsurance of 20% or more of the drug cost leading to out-of-pocket expense for patients exceeding $3,000 per year. Co-pays and coinsurance drug costs paid by patients lower PBM drug reimbursement amounts, and these savings are reflected in lower overall drug costs for employers.

### Inefficiencies in Healthcare Delivery and Treatment Models

Two broad sets of inefficiencies are observed. One is clinical inefficiency, and the other is healthcare delivery inefficiency. A set of novel processes can be used to address these inefficiencies. Improvement in efficiency will typically be removal of some aspects of inefficiencies, which may result in greater speed, faster treatment response, lesser cost, fewer mistakes, better economic outcomes, etc.

Achieving excellent clinical outcome, e.g., excellent therapeutic response or clinical remission of the disease, ultimately leads to a better economic outcome for payers, employers, pension funds, and patients. One can achieve excellent clinical outcome either on a per patient basis or on a subset(s) of patients basis, which will translate to better economic outcomes for the corresponding payer, employer, and the said patient. Alternatively, one can target an entire disease population that comes under a single payer or provider system by way of developing and administering disease-specific PDPs. Examples of such disease populations are rheumatoid arthritis, multiple sclerosis, as well as specific oncology indications such as B-NHL, CLL, etc. Yet another example is to provide excellent clinical outcome for 10-20%, or 30-60%, or 80% of a particular disease population, e.g., rheumatoid arthritis.

Clinical outcome is a function of therapeutic efficiency and treatment efficiency. Therapeutic efficiency determines whether a drug is clinically and therapeutically effective in a given patient or subset(s) of patients. The way a patient is treated with a drug refers to treatment (or therapy) efficiency. Examples include monotherapy versus combination therapy. Monotherapy could be, e.g., a small molecule therapy or antibody therapy. An antibody therapy along with chemotherapy is an example of combination therapy. Administering the drug as a single course versus several times at defined time points over several months to years, referred to as maintenance therapy, is another example. Clinical inefficiency can be addressed by providing theragnostic guidance—selection of a drug for treatment and guiding the treatment protocols such as dosing, schedule, etc.

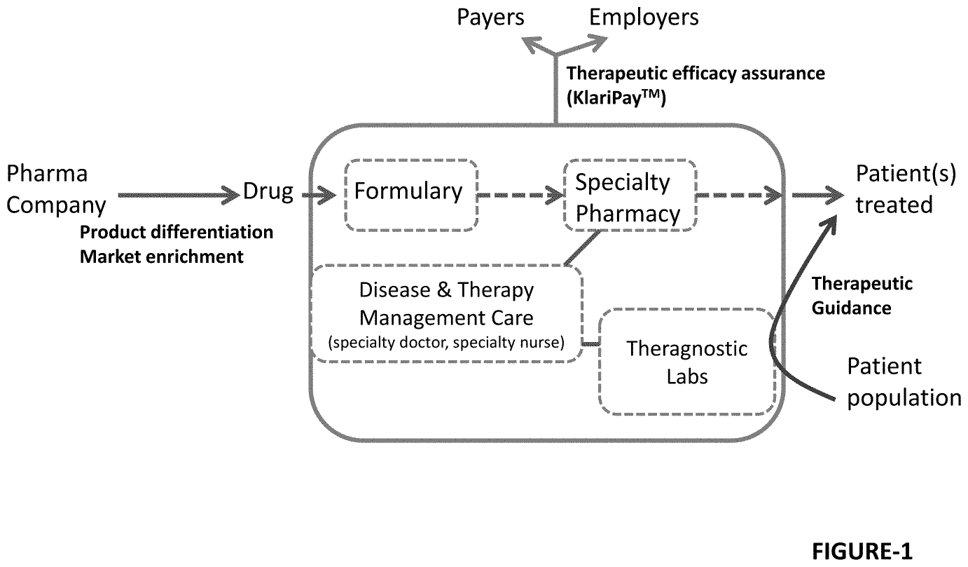

Healthcare delivery inefficiency is addressed through three novel features: (a) therapeutic guidance based on evaluation of the theragnostics; (b) efficacy assurance (KlariPay™); and (c) product differentiation for a specialty drug. All three features constitute the Klaritos delivery and treatment platform (FIG. 1).

Therapeutic Guidance Based on Theragnostics:

This involves selection of a specialty drug from a panel of marketed specialty drugs for a subject or a patient subset(s); selection of a treatment regimen (single course versus maintenance therapy; monotherapy versus combination therapy; or simply a ‘watch and wait’ regimen in the case of B-NHL).

Such theragnostic methods are mechanism-driven: (a) based on the mechanism of action by which the drug exerts therapeutic response in an individual or in individuals having the appropriate or desired genetic or immunological makeup, and by determining whether the patient will then respond to that therapy or not; (b) based on the disease severity mechanisms, patient populations can be stratified and appropriate specialty drugs are then administered selected to achieve better clinical responses, preferably clinical remission.

It also involves continued, systematic monitoring of disease remission and relapse patterns during the course of administration to ascertain how well the drug is working (or not working) in a given subject or subset(s) of subjects, and when to administer the next course of therapy (e.g., as-needed versus fixed time intervals). These continued monitoring or evaluating is part of the theragnostic evaluation, or evaluation of diagnostic measures for theragnostic purposes.

Efficacy Assurance (KlariPay™):

Efficacy assurance consists of therapeutic efficacy assurance (TEA) and financial assurance (FA). TEA refers to an assurance of achieving significantly better therapeutic efficacy in a given patient, within a reasonable time-frame, e.g., 1 month, 2-3 months; this assurance is provided to patients, or payers, and employers. Specifically, TEA is not therapeutic risk assurance; that is, this does not cover risks and side effects associated with the drugs. Financial assurance refers to a form of money-back guarantee, e.g., co-insurance amount, if the therapy has not achieved desired therapeutic outcome within a reasonable time-frame, e.g., 2-3 months; this assurance is provided to patients, and in some instances, it may also be provided to payers and employers. Such assurance is theragnostics-guided in specific disease indications, provided the patient establishes and maintains excellent patient therapeutic adherence rate. Both TEA and FA are inter-related: it is essentially a warranty that some or all of the cost of drug, with or without treatment costs, will be returned if the patient does not achieve a designated treatment response provided the therapy plan is carefully adhered to by the patient. Payment for the specialty drug is tied to the therapeutic efficacy in a patient. This feature may provide (a) time savings and (or) cost savings for the patients and payers, and (b) appropriate therapies can be selected and administered on time, and (c) equally importantly, unnecessary treatments that might be less efficacious in a subject need not be administered at the first place. Beneficiaries of KlariPay include: patients, payers, specialty drug company, and Klaritos platform that includes the theragnostic provider and the DTM care provider. KlariPay is also the theragnostics-guided platform that enables the efficient delivery, treatment, and payment for specialty drugs.

In one embodiment, without limiting the scope of the definition, excellent therapeutic efficacy would be: disease remission, relapse-free survival, significantly extended progression-free survival, etc. For instance, in rheumatoid arthritis, subject achieving ACR 70 criteria is considered excellent therapeutic efficacy. In one instance, the payment is made to the specialty pharmacy by the payers (herein payers mean government or private payers, employers, pension funds, and patients) immediately upon dispensing the drug. In another instance, the payment is made by the payers during the treatment period, e.g., 2-3 months after the initiation of treatment, provided the expected, pre-approved excellent (or good) clinical outcome is achieved as determined by theragnostic guidance. This electronic financial transaction occurs through KlariPay, preferably instantly, e.g., in the order of hours or days.

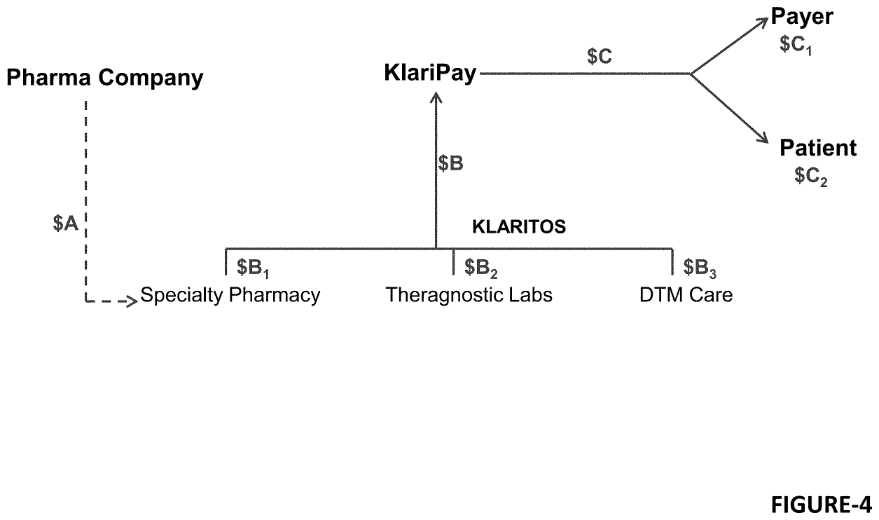

If the patient does not achieve clinical remission (or excellent response or such pre-defined criteria depending on the disease and stage of the disease etc.) after confirmation of compliance with drug regimen, the specialty drug company and the theragnostic guidance provider agree to pay the money back to payers, minus the pre-agreed applicable costs for goods and services rendered by the specialty drug company and the theragnostic provider (FIG. 4). Provided that an alternate therapy is available in Klaritos formulary, upon prior authorization, the patient will be treated with that drug at the earliest. In one embodiment, without limiting the scope of the definition, not achieving clinical remission may mean poor response, partial response, moderate response, poor progression-free survival, poor event-free survival, disease relapse, etc. For instance, in rheumatoid arthritis, this means the subject does not achieve ACR70 criteria. This electronic financial transaction occurs through KlariPay, preferably instantly, e.g., in the order of hours or days. In one embodiment, the payment is made to the payers as per the pre-agreed terms; in this context, the term payers may include employers and patients.

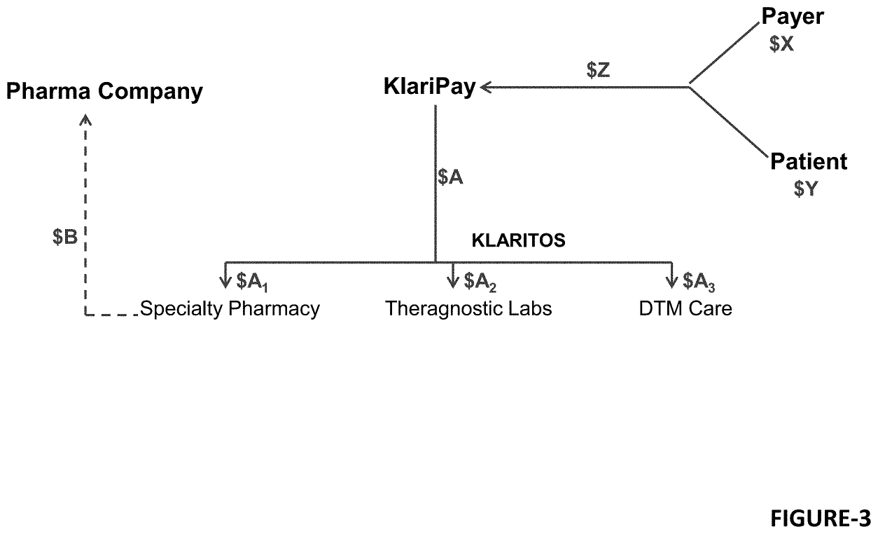

Conversely, if the patient achieves clinical remission (or excellent clinical response), the payer(s) agrees to pay a higher price for the specialty drug (FIG. 3). For example, if the annual drug price is $50,000, then the payer agrees to pay, e.g., additional $15,000 (30%) through KlariPay. Specialty drug provider and the theragnostic guidance provider may split this additional 30% payment, e.g., in equal halves. Alternatively, assuming the patient is in remission during the second year after the administration of the specialty drug, the payer will pay, e.g., additional $50,000 through KlariPay. Specialty drug provider and the theragnostic guidance providers will split this $50,000 payment, e.g., in two equal halves. Notwithstanding these examples, additional incentives to the theragnostic provider (e.g., PDP, PBM, specialty pharmacy) can be envisioned.

In one instance, the current standard of care for treating a B-NHL patient (stage-2 disease) may involve rituximab+CHOP combination therapy. However, if Klaritos achieves excellent response in the subject by administering only rituximab therapy (and withholding CHOP therapy), payer has to pay additional payment, e.g., $25,000 per year, through KlariPay. The theragnostic payer may or may not have the reason to share this incentive with the specialty drug company.

In yet another instance, e.g., the current standard of care to treat a rheumatoid arthritis patient is to first treat with methotrexate, and upon failed treatment, treated with infliximab, and then with etanercept, and then with adalimumab, and then with tocilizumab. In this scenario, the patient has spent nearly 5-15 years before he could find the right treatment that works, and during this period, the disease progression is extremely severe involving several surgeries. In fact, the patient has lost the effective window-of-opportunity-to-treat, just because even the therapy, e.g., tocilizumab, that might work for that patient might not be efficacious anymore, given the severity of the disease. This is huge economic burden for payer and the patient. Through the Klaritos approach, however, theragnostic guidance establishes that the patient is eligible for tocilizumab therapy as the second line of treatment, immediately after failed methotrexate treatment, and the patient responds well to tocilizumab therapy and goes into remission. This saves approximately 5-10 years of trial-and-error-treatment-finding procedure for the patient. This is a significant paradigm shift in standard of care, and thus, payer has to pay additional payment, e.g., $100,000, through KlariPay, and it may not have the reason to share this incentive with the specialty drug company.

Product Differentiation for a Specialty Drug:

When a specialty drug enters the market through this proprietary healthcare supply chain and delivery model, because of the therapeutic guidance the drug will have, the drug is most probably expected to differentiate itself from other IP-protected drugs as well as its biosimilars or generics in the market in regards to efficacy, safety and toxicity profiles. Let us assume that the drug is eligible for treatment in 25% of the total disease population, while the market size comes down, more patients from this market size may be administered and thus this leads to enhanced market share, e.g., 2-4 fold. Thus, this equates to market enrichment, and the specialty drug is expected to have nearly the same amount of net sales as it would have in an all-corners market. We refer this as theragnostics-guided product differentiation strategy. If the drug achieves increased net sales because of the strategy, the specialty drug company agrees to pay royalty to Klaritos platform, and such royalty is tiered, for e.g., anywhere from 2% to 70% of the net sales of the drug in that market. Alternatively, Klaritos platform will receive payments from the specialty drug company based on the pre-negotiated contingent value rights (CVR).

The United States healthcare system, and counterpart healthcare systems throughout the world, is very complex. Besides involving a significant portion of the economy, many stakeholders have conflicting, vested interests, not only in the general operation of the system, but in maintaining their vantage positions. Background on operation of the system can be found, e.g., in Kovner, et al. (2015) Jonas and Kovner's Health Care Delivery in the United States (11th Edition) Springer ISBN-10: 0826125271, ISBN-13: 978-0826125279; and Barton (2009) Understanding the US Healthcare System (4th Edition) Health Administration Press ISBN-10: 1567933386, ISBN-13: 978-1567933383. Background in health policy and law can be found, e.g., in Teitelbaum and Wilensky (2016) Essentials of Health Policy and Law (Essential Public Health; 3d ed.) Jones and Bartlett Learning ISBN-10: 1284087549, ISBN-13: 978-1284087543; Longest (2015) Health Policymaking in the United States (6th edition) Health Administration Press, ISBN-10: 1567937195, ISBN-13: 978-1567937190; Estes, et al. (2012) Health Policy: Crisis and Reform (6th Edition) Jones and Bartlett Learning, ISBN-10: 076379788X, ISBN-13: 978-0763797881; Hall, et al. (2013) Health Care Law and Ethics (8th ed.) Aspen Pub. ISBN-10: 0735507112, ISBN-13: 978-0735507111; and Committee on Engineering and the Health Care System, Institute of Medicine (2005) Building a Better Delivery System: A New Engineering/Health Care Partnership National Academies Press, ISBN-10: 0309386705, ISBN-13: 978-0309386708. Periodicals or journals which publish on these topics include, e.g., Health Affairs, “at the intersection of health, health care, and policy”, see www.healthaffairs.org; Medical Care Research and Review, see sage journals, mcr.sagepub.com; American Journal of Managed Care, managed markets network, see www.ajmc.com; American Journal of Public Health, publication of the American Public Health Association, see http://ajph.aphapublications.org/; and Journal of Health Politics, Policy and Law, from Duke University Press, ISSN: 0361-6878, e-ISSN: 1527-1927; see http://jhppl.dukejournals.org/.

### Specialty Drugs: Current Approval, Dispensing, and Payment Processes

Managed care organizations and pharmacy benefit managers (PBMs) serve either through commercial or government payers to control or slow the rate of cost increases while ensuring a reasonable level of patient care. As specialty drugs and other innovator brand biopharmaceutical manufacturers have implemented significant price increases on their products year over year, and as specialty drugs have become an ever-larger part of pharmaceutical spending, PBMs have resorted to a number of measures to manage utilization and control costs.

Utilization management is implemented through a drug benefit design developed by PBMs as part of the prescription drug plan (PDP). It consists of a formulary with multiple tier designation for drugs (generic, innovator brand, non-preferred brand and specialty tiers). Drugs listed on innovator brand and non-preferred brand tiers have significantly higher patient co-payments than the generic drug tier, and the specialty drug tier requires a patient to pay co-insurance or a percentage of the cost of the drug. The goal of higher (co-pays) co-insurance is to steer patients to lower cost alternatives.

Innovator brand companies help off-set the cost of drug co-payments and co-insurance for patients covered through commercial insurers by offering co-pay cards and covering the cost of co-insurance for patients who are income-eligible. Medicare patients can obtain coverage through charitable organizations many of which are funded by pharmaceutical companies.

Other measures to manage utilization within the formulary framework include: (a) Step Therapy where a patient is required to try a lower cost alternative or generic pharmaceutical, if available, before they can receive a higher cost brand drug, (b) Prior Authorization where a physician must document the medical reasons (medical necessity) for a patient to achieve such a particular therapy. This approach is widely used for most specialty drugs, and (c) Quantity Limits wherein the patient may receive a prescription for a smaller quantity, such as one-week or one-month supply of an expensive medication at any given time.

### Drug Formulary: Design and Management

PBMs maintain a formulary committee consisting of credentialed pharmacists and physicians qualified in various subspecialties (i.e., neurology, oncology), experts in health economics and relevant business people. When an innovator drug is approved by the FDA, the committee will assign one or more individuals within the group to review all published data on the product including any comments from FDA (advisory committee) about the product label, and consider the product in the context of other therapies currently available. The manufacturer will provide a dossier on the product to supplement the review which will include certain unpublished data and the wholesale acquisition price.

Based on such product reviews, a PBM will develop a policy describing guidelines for coverage of the product. These guidelines are added to existing drug formulary information and electronically communicated to specialty pharmacies within the PBM network. Usually physicians become aware of the guidelines through biopharmaceutical sales representatives and reimbursement specialists who work for the company.

PBMs have the delicate task of maintaining satisfaction among their key stakeholders each of whom have diverse needs and expectations: employers who ultimately pay the cost for prescription drugs and want to control cost; employees (patients) who use the prescription drug plan and want the most effective drug at the lowest co-insurance cost to them; and physicians who determine the appropriate medication within the guidelines of the formulary of the PDP who want to have broad discretion on what they can prescribe. In general, PBMs do not try to dictate the usage of particular medicines but steer the utilization towards the least costly but most effective option(s).

Self-Administered Drugs

1. Physician evaluates patient and decides on a specialty drug.
2. Office staff/physician checks guidelines for use of therapy based on patient's PDP and formulary, or works with reimbursement specialists at the biopharmaceutical company or a third-party organization retained by the biopharmaceutical company to obtain financial support. This may also include the need to obtain information through companion diagnostic testing prior to prescribing the drug.
3. Office staff submits prescription to PBM which is approved or denied based on PBM prior approval process/medical policy guidelines. If prescription is denied, physician must complete a special medical necessity form to obtain approval.
4. Once prescription is approved, office staff of physician or patient works with reimbursement support specialists to determine if patient is eligible for co-insurance assistance from the biopharmaceutical company. If the patient is covered by Medicare, assistance may be available through various charities.
5. After co-insurance assistance is determined, physician's office contacts an approved specialty pharmacy (SP) in the PBM network where the patient has drug coverage and transmits prescription electronically or by fax.
6. The specialty pharmacy confirms electronically that the prescription has been approved by the PBM.
7. Patient calls SP and pays all or partial co-insurance cost or reimbursement support specialists contact SP and make payment on behalf of biopharmaceutical company. Alternatively, a SP gets authorization from a charitable organization.
8. SP dispenses or delivers drug to the patient.
9. SP submits a claim for drug reimbursement to the PBM after deducting the amount of patient's co-insurance.
10. PBM reimburses SP, and submits a separate charge to the employer of the patient or Medicare depending on the coverage for payment of the drug cost.

### Drugs Infused in a Physician's Office

A. “Buy and Bill”, Physician's buy the drug from distributors (for e.g., McKesson, Cardinal Health, AmerisourceBergen), and submit charges to the commercial insurers or government payer PBMs
See steps 1-4 under section on self-administered drugs.
5. Physician's office collects co-insurance from patient or from biopharmaceutical company or charitable organization depending on the types of coverage and income eligibility.
6. Physician administers drug to the patient.
7. Physician submits a claim for drug reimbursement to the patient's PBM after deducting the amount of the patient's co-insurance for commercially insured patients.
8. PBM reimburses physician's office and submits a separate charge to the employer of the patient for payment of the drug cost.
9. For Medicare patients, physicians submit the claim directly to Centers for Medicare and Medicaid Services (CMS) for reimbursement.
B. “White Bagging,” SP sends drug to physician's office for administration See steps 1-7 under section on self-administered.
8. SP ships drug to physician's office where it is administered to the patient.
9-10. See steps 9-10 under self-administered drugs.
11. For Medicare patients, SP submits a claim directly to CMS for reimbursement.
C. Brown Bagging,” SP sends drug to the patient who takes to the physician's office for administration
All other steps are the same as in “white bagging”.

Unless defined otherwise, all technical and scientific terms used herein have the same meaning as commonly understood by one of ordinary skill in the art to which this invention belongs. Accordingly, the following terms are intended to have the following meanings:

“Subject,” “individual, “host,” or “patient” generally refers to humans. As used herein, “subject,” “individual, “host” or “patient” includes one who is to be tested, or has been tested for prediction, assessment, diagnosis, theragnostics of a disease or disorder to be treated, wholly or partially, with a specialty drug.

“Small molecule” drug refers to a pharmacologically active compound, e.g., as metabolized, having a molecular weight of less than about 1000 daltons, and typically between 300 and 700 daltons. Most drugs are small molecules, administered orally. Examples of small molecule drugs are tofacitinib and sofosbuvir.

“Significant” in the context of a measure, e.g., therapeutic or economic measure, e.g., in a difference in efficiency or response, will generally mean a number which can be objectively determined with some accuracy, and in the context is measurable and easily detectable. In most circumstances, e.g., it may be at least about 3%, 6%, 9% or more, and more preferably at least in the 10-15% or more range, as much as about 20% to 30% or more. The measure may refer to either an individual measure, averaged over a group, or measured over appropriate comparison groups. In many situations, the effects may be more easily or only identified in certain subsets or segments of the patient pools compared to others.

“High” or “highly” will typically be at least significant, and will be a measure greater than threshold for statistically significant. Preferably it will be about 1.5 to 2X, whether on an individual or patient group basis, but which may be readily detectable in only certain subsets.

“Eligible” or “qualifying” is meant to refer to something which otherwise is within a category of passing initial screening criteria. Thus, an eligible patient or subset will be a patient or subset who initially is considered within the class of patients for whom the drug or treatment is considered appropriate. In some embodiments, an eligible patient, payer, or employer in the context of assurance (therapeutic or financial) is one who qualifies according to the terms of the assurance, who has complied with the terms to an acceptable degree, e.g., patient therapeutic adherence, or timely payments by the payer or employer.

“Product differentiation” herein refers to differentiation, e.g., theragnostics-guided, of a drug from another, commercially available drug(s) for treatment of a particular disease or cancer. One of the objectives being achieving better therapeutic and economic outcomes. Such differentiation can lead to selection of that particular drug instead of other intellectual property-protected drugs, or its biosimilars or generics. Product differentiation can enhance efficacy in patient subsets or segment(s), therapeutic value, economic value, financial value, or better pricing. This feature may be exploited by (a) a prescription drug plan, (b) a drug formulary, (c) a specialty pharmacy, (c) a payer, (d) an employer, (e) a pharmaceutical company, (f) a diagnostic company, (g) a drug distributor, or (h) a healthcare provider.

“Market enrichment” herein refers to identification, e.g., theragnostics-guided, of a treatable patient, treatable subset(s) of patients, a treatable segment of patient market in a particular disease indication for the purposes of distribution, delivery of a drug, and treatment with a drug, with an objective of achieving better therapeutic and economic outcomes. This method selectively avoids patients who are considered not eligible for a particular therapy. This market enrichment feature may be exploited by (a) a prescription drug plan, (b) a drug formulary, (c) a specialty pharmacy, (c) a payer, (d) an employer, (e) a pharmaceutical company, (f) a diagnostic company, (g) a drug distributor, or (h) a healthcare provider.

“Prescription Drug Plan” herein refers to a drug plan managed and administered by a PBM. For instance, it can be a disease-specific PDP consisting of specialty drugs and non-specialty drugs that are selected by a theragnostics-guided strategy. An exemplary disease-specific PDP targets rheumatoid arthritis.

“Antibody” refers to an immunoglobulin or fragment thereof, and encompasses any such polypeptide comprising an antigen-binding fragment of an antibody. The term includes but is not limited to polyclonal, monoclonal, monospecific, multispecific (e.g., bispecific antibodies), humanized, human, single-chain, chimeric, synthetic, recombinant, hybrid, mutated, grafted, antibody fragments (e.g., a portion of a full-length antibody, generally the antigen binding or variable region thereof, e.g., Fab, Fab′, F(ab′)2, and Fv fragments and in vitro generated antibodies so long as they exhibit the desired biological activity).

“Antibody therapy” refers to a medical treatment involving an antibody. An “antibody therapy” in reference to an ADCC-treatable disease refers to an antibody that has a therapeutic mechanism based wholly or in part on ADCC.

“Biosimilar” herein refers to a biological drug, e.g., an antibody such as adalimumab, that is structurally (i.e., gene and amino acid sequences; glycosylation and post translational modifications all combined) and functionally (i.e., therapeutically, immunologically, pharmacologically, etc.) similar or identical, but not necessarily identical to the original biological drug that is referred to as reference product. Intentional or unintentional changes, e.g., amino acid changes or glycosylation heterogeneity, may or may not be present in biosimilars. Biosuperiors and biobetters are biosimilars. A biosimilar may not be therapeutically equivalent to its reference product.

“Bioequivalent” herein refers to a biological drug that is structurally an exact copy of the reference product, and thus therapeutically and functionally expected to be equivalent, e.g., not significantly better or worse than the original molecule, i.e., reference product. Minor glycosylation heterogeneity with little or no impact on therapeutic efficacy can be observed in bioequivalents. A bioequivalent can be a biosimilar but not all biosimilars are bioequivalents.

“Biosimilar substitution” herein refers to the process by which an FDA-approved, interchangeable biosimilar product may be substituted for the prescribed biological product, e.g., a reference product developed by the innovator. Patients or physicians or payers or all of the above may have to be notified of the substitution. Under applicable provisions, payers, PBMs, or specialty pharmacies can authorize and (or) initiate substitution, e.g., preauthorized or authorized substitution. If the drug is interchangeable, it may be substituted (interchanged) for the reference product without the intervention of the healthcare provider who prescribed the reference product.

“Biosimilar extrapolation” refers to approval, prescription, and administration of a biosimilar in other disease indications, though typically a clinical trial in that particular disease indication is not conducted. For instance, a reference product may have been approved in multiple disease indications. This extrapolation is based on the premise that if a biosimilar, preferably a bioequivalent, is shown to be comparable (e.g., indistinguishable) to the reference product in one disease indication in regards to safety and clinical efficacy, then it is expected to work similarly in other approved indications as that of the reference product.

“Substitution” herein refers to the prior authorization, dispensing and delivery, and treatment of a disease indication in the patient with another drug, e.g., specialty drug, non-specialty drug, that is other than the originally prescribed drug by the patient's disease specialist. Substitution will typically be approved by the prescribing physician, but in certain jurisdictions and appropriate situations, may be substituted without such when permitted.

“Isolated cells” refers to a preparation of cells that have been separated from other components in a mixture containing the cells. In some embodiments, the cells are in the form of a “substantially purified” cell preparation, e.g., containing substantially lesser amounts of extraneous cells or materials.

“Genotype” refers to the alleles present in DNA from a subject or patient, where an allele can be defined by the particular nucleotide(s) present in a nucleic acid sequence at a particular site(s). Often a genotype is the nucleotide(s) present at a single polymorphic site known or found to vary in the population. In some embodiments, a “genotype” is reflected in an expressed protein, which may be detected by known procedures, such as by using antibodies or protein sequencing.

“Polymorphism” refers to the coexistence of more than one form of a gene or portion thereof. While a polymorphism is present at the nucleotide level, it may also manifest in an expressed gene product, e.g., a protein.

“Allele,” which is used interchangeably herein with “allelic variant” and “variant allele”, refers to alternative forms of a gene or portions thereof. Alleles occupy the same locus or position on homologous chromosomes. When a subject has two identical alleles of a gene, the patient is said to be homozygous for the gene or allele. When a subject has two different alleles of a gene, the patient is said to be heterozygous for the gene. Alleles of a specific gene, including FcγRIIA, can differ from each other in a single nucleotide, or several nucleotides, and can include substitutions, deletions, and insertions of nucleotides. An allele of a gene can also be a form of a gene containing one or more mutations.

“Fcγ receptor polymorphism” refers to more than one form of a gene for a specific Fcγ receptor. By an FcγRIIA polymorphism, it is meant a polymorphism in the FcγRIIA gene which results in an amino acid substitution in the FcγRIIA protein. By an FcγRIIIA polymorphism, it is meant a polymorphism in the FcγRIIIA gene which results in an amino acid substitution in the FcγRIIIA protein.

“Amino acid residue” and “amino acid position” are used interchangeably herein to refer to the position of the specified amino acid in the polypeptide chain. In some embodiments, the amino acid residue can be represented as “X^{N}”, where X represents the amino acid and the N represents its position in the polypeptide chain. Where two or more variations, e.g., polymorphisms, occur at the same amino acid position, the variations can be represented with a “/” separating the polymorphisms. For example, two possible polymorphisms can be represented as “X/Y^{N}”, where X and Y represent the possible amino acids and N represents the position in the polypeptide chain. In some embodiments, the two possible variations can also be represented as “XNY”, where X, N and Y are as described above, e.g., H131R.

“Induction therapy” refers to the first course of treatment in treating a disease, disorder or medical condition.

“Maintenance therapy” refers to a therapy, therapeutic regimen or course of therapy which is administered subsequent to an induction therapy (an initial course of therapy administered to an individual or subject with a disease or disorder). As used herein, therapy that includes maintenance therapy is included as comprising maintenance therapy. Maintenance can be used to halt or reverse the progression of the disease/disorder), to maintain the improvement in health achieved by induction therapy and/or enhance, or “consolidate”, the gains obtained by induction therapy.

“Antibody maintenance therapy” refers to an antibody therapy, i.e., a therapy comprising one or more antibodies, which is administered as maintenance therapy in the therapeutic regimen or course of therapy.

“Responsiveness” in reference to a subject refers to a treatment outcome or a clinical outcome of a treatment or therapy for a disease or disorder. The treatment outcome or clinical outcome can be measured according to standards recognized in the art for a specific disease or disorder.

“Predicting” refers to determining the probability or likelihood of a particular outcome or event. In reference to responsiveness to treatment, the term refers to the likelihood of a particular treatment outcome or clinical outcome.

“Predicting responsiveness”, or “providing a prognosis” or “prognosing”, it is meant predicting whether or not the antibody maintenance therapy will have an impact on disease progression.

“Overall survival” or “OS” refers to the time (in years) measured from diagnosis, study entry, or early randomization (depending on the study design) to death from any cause. Overall survival is a term that denotes the chances of staying alive for a group of individuals suffering from a disease or disorder.

“Progression free survival” or “PFS” refers to the time (in years) measured from the start of maintenance therapy during which the disease being treated does not worsen. Progression free survival is a metric that denotes the chances of a disease stabilizing or being reversed in a group of individuals suffering from the disease. For instance, it denotes the percentage of individuals in the group who are likely to be as healthy if not healthier after a particular period of time following the start of maintenance therapy.

“Relapse-free survival” or “RFS” refers to the time (typically in years) measured from diagnosis to first recurrence of the disease, e.g., first recurrence of a malignancy in a neoplastic disease. RFS is defined only for patients achieving complete remission, and is measured from the date of achievement of a remission until the date of relapse or death from any cause.

“Event-free survival” or “EFS” refers to the time (typically in years) measured from diagnosis to the first subsequent event associated with the disease, e.g., complications from the disease, first malignancy recurrence, or death. EFS is defined for all patients of a trial, and is measured from the date of entry into a study to the date of induction treatment failure, or relapse from complete remission (CR) or CRi, or death from any cause.

“Time to Progression” or “TTP” refers to a measure of time after a disease is diagnosed (or treated) until the disease begins to worsen.

“Chemotherapy” or “chemotherapeutic regimen” refers to the administration of at least one chemotherapy agent that is used to treat a disease or disorder. Chemotherapy agents may be administered to a subject in a single bolus dose, or may be administered in smaller doses over time. A single chemotherapeutic agent may be used (single-agent therapy) or more than one agent may be used in combination as combination therapy. A chemotherapeutic agent as used herein comprises a non-biologic therapeutic, including small molecule drugs, peptide drugs, anti-sense nucleic acids, etc.

“Administering an antibody therapy” or “administering an antibody maintenance therapy” refers to administering an antibody to a subject for purposes of therapy (e.g., induction therapy) or maintenance therapy, respectively.

“Administered regularly” refers to administration of a therapeutic (e.g., drug or biologic) or treatment at periodic intervals.

“Administered as-needed” refers to administration of a therapeutic (e.g., drug or biologic) or treatment when the subject suffers a relapse or a diagnostic measure indicates the need for retreatment (e.g., target cell repopulation), and is generally determined by a medical doctor of skill in the art. This may involve continued monitoring of the patient, e.g., daily, weekly, monthly, etc., in regards to her disease state.

“Course of treatment” or “course of therapy” refers to administration of a drug or therapeutic for a period of time as part of a defined treatment plan. The course of treatment or therapy can be a first course, second course, third course, etc. The courses may or may not use the same therapeutic. The drug or therapeutic can be administered as a single dose or in multiple doses in a single course. Multiple doses in a course of therapy can be administered over a period of time, such as days, weeks or months, depending on the therapeutic and the disease or disorder to be treated. Subsequent treatment strategies may be adjusted according to previous treatment response or disease progression, remission, or relapse patterns.

“Differential dosing” refers to the selection and/or administration of a treatment regimen in which the dose of an active pharmaceutical ingredient (e.g., drug or biologic) is altered to optimize for efficacy and/or tolerance in the treatment of a subject. The active pharmaceutical ingredient for which the dose is altered can be in the form of a monotherapy or as a component in a combination therapy.

“Differential dosing schedule” refers to the selection and/or administration of a treatment regimen in which the length of time the patient is treated is altered to optimize for efficacy and/or tolerance in the treatment of a subject. In some embodiments, differential dosing schedule includes a form of maintenance therapy.

“Differential dosing frequency” refers to the selection and/or administration of a treatment regimen in which the frequency of administration or dosing cycle is altered to optimize for efficacy and/or tolerance in the treatment of a subject.

“Step therapy” or “fail first” or “fail-this-one-first therapy” refers to a process an insurance company requires the patient to go through first and fail a medication or service preferred by the insurance provider, typically considered more cost effective, often on the short term, or safer, before the insurance company will cover a different drug or service. Unless absolutely necessary, majority of specialty drugs are not currently favored by PBMs as the first therapy particularly when less-expensive therapies or treatment modalities are available.

“Personalized medicine” refers to methods of identifying the right patient(s) for the right therapy. The patient may have a characteristic genotypic and (or) phenotypic feature(s) and such features are mechanistically relevant for achieving better, e.g., excellent response or remission, therapeutic efficacy when an appropriate therapy is administered. Such mechanistic features may involve better binding of the drug, better mechanism of action of the drug, better cell killing of specific cell types, etc. For instance, in antibody therapies, ADCC is one such mechanism of action that is linked to genetic polymorphisms in patients.

In contrast, individualized medicine or precision medicine, as used herein, contemplates the longitudinal and temporal disease states of the individual; the matching of the therapy to the individual will typically include evaluating the changes in that individual with time in regards to: disease progression, remission, relapse patterns, and other physiological factors which affect the disease state. Thus, individualized medicine is a more temporally-based matching of treatment to the current state of the individual with the main objective of achieving better treatment and economic outcomes. Theragnostic methods guide such individualized or precision medicine.

Matching an appropriate drug to an appropriate individual patient is selecting a combination that both are correct, i.e., both the drug to the patient, and the patient to the drug. Sometimes there will be multiple matches, in which case, certain pairings will be preferred for various reasons, whether medical, convenience, practical, economic, or other reasons. Theragnostics methods help guide such matching.

“Stratifying” or “stratification” refers to classifying subjects into distinct groups based common characteristic(s) or trait(s). Stratification can be based on a single trait or two or more traits, e.g., of disease presentation. When the occurrences of two or more characteristics or traits are statistically linked, one of the traits can be stratified based on the other trait. For example, when the genotype and responsiveness to treatment or therapeutic regimen are linked, responsiveness can be stratified or classified based on the genotype.

“Reference stratification” as used herein refers to an established stratification scheme that has stratified a treatment response/clinical outcome-genotype association, with statistically significant differences between the different groups in the stratification. Accordingly, a subject afflicted with an ADCC treatable disease whose genotype for the Fcγ receptor polymorphism (e.g., FcγRIIA and/or FcγRIIIA), is known can be compared to the reference stratification to identify the likelihood of the subject having a particular treatment outcome or clinical outcome, i.e., responsiveness, for an antibody maintenance therapy.

“Correlating,” “correlation,” “correlates,” as used herein refer to the establishment of a relationship, e.g., mutual or reciprocal, between, e.g., genotype status and therapeutic efficacy of certain treatments as described herein. That is, correlating may refer to relating the genotype status to responsiveness to treatment or therapy.

“Excluding a treatment or therapy” refers to removing a possible treatment from consideration, e.g., for use on a particular patient, based on the presence or absence of a particular variance(s) in one or more genes of that patient. This typically means the treatment or therapy is counter-indicated or inappropriate for the particular patient.

“Excluding a subject” refers to removing the subject from consideration of a treatment or therapy, including in reference to treatment or therapy in clinical trials, based on the presence or absence of a particular variance(s) in one or more genes of that patient. This typically means the patient is therapeutically ineligible for such treatment or therapy.

“Selecting a treatment or therapy” refers to including a possible treatment for consideration, e.g., for treating a particular patient based on the presence or absence of a particular variance(s) in one or more genes of that patient. Such a treatment or therapy is considered an option for the patient, though some options may be of higher or lower appropriateness, depending upon the specific criteria being applied based on theragnostic methods.

“Selecting a subject” refers to including the subject for consideration of a treatment or therapy, including in reference to treatment or therapy in clinical trials, based on the presence or absence of a particular variance(s) in one or more genes of that patient.

“Companion diagnostics” refers to devices or tests that provide information that is essential (required) for the safe and effective use of a corresponding therapeutic product, typically linked to a specific drug within its approved labeling. Others refers this to determining suitability of patients for tailored or targeted forms of therapy. Currently, these tests do not provide efficacy or financial assurances.

“Complementary diagnostics” refers to tests intended but not required to indicate whether a patient should be treated with certain therapies rather than one particular drug. Currently, these tests do not provide efficacy or financial assurances.

Clinically, biomarkers are commonly used for diagnostic (disease identification) and prognostic (predicted outcome or progression) purposes. A theranostic biomarker could identify the most appropriate treatment for an individual, indicate the correct dose, or predict response to treatment. This approach attempts to maximize drug efficacy, minimize toxicity and provides a more informed treatment choice (for physicians and patients). Perhaps for a theranostic biomarker to be truly clinically useful, it should retain predictive value for response irrespective of the methods used to assess improvement in disease activity. Currently, these tests do not provide efficacy or financial assurances.

“Neoplastic disease or disorder” refers to a disease state in a subject in which there are cells and/or tissues which proliferate abnormally. Neoplastic disorders can include, but are not limited to, cancers, sarcomas, tumors, leukemias, lymphomas, and the like. Hyperproliferative disorders, or malignancies, are conditions in which there is at least some element of unregulated cell growth. The terms “cancer,” “neoplasm,” “hyperproliferative cell,” and “tumor” are used interchangeably herein to refer to cells which exhibit relatively autonomous growth, so that they exhibit an aberrant growth phenotype characterized by a significant loss of control of cell proliferation. Cancerous cells can be benign or malignant. Viral infections (e.g., HCV infection in B-cells) can lead to hyper(lympho)proliferative disorders.

“Autoimmune disease or disorder” refers to a disease state or condition caused by immune-responsiveness against self-tissues and/or substances normally present in the body. It is generally associated with production of inflammatory factors, which further promote tissue destruction and disease progression. Inflammatory macrophages, inflammatory NKT cells, etc., can cause chronic inflammatory diseases such as atherosclerosis, Type-2 diabetes, sickle cell disease, and the like. Autoimmune diseases can be systemic or organ-specific. Examples of systemic autoimmune diseases include: multiple sclerosis, rheumatoid arthritis, systemic lupus erythematosus, ankylosing spondylitis, scleroderma and Sjögren's syndrome. Examples or organ-specific autoimmune diseases include: Addison's disease, Autoimmune hemolytic anemia, Goodpasture's syndrome, Grave's disease, Hashimoto's thyroiditis, idiopathic thrombocytopenia purpura, insulin-dependent diabetes mellitus, myasthenia gravis, pernicious anemia, poststreptococcal glomerulonephritis and psoriasis.

“Inflammatory disease or disorder” refers to a disease or disorder caused by or resulting from or resulting in inflammation. The term “inflammatory disease” may also refer to a dysregulated inflammatory reaction that causes an exaggerated response by macrophages, granulocytes, and/or T-lymphocytes leading to abnormal tissue damage and cell death. In some embodiments, an inflammatory disease or disorder can be an aspect of other diseases, such as autoimmune diseases.

“Microbial infections” refers to a disease or disorder caused by or resulting from a microbial infection. Microbial infections refer to diseases caused by bacteria, fungi, viruses. Examples include infections by hepatitis C virus (HCV), or human immunodeficiency virus (HIV).

“Allograft rejection” refers to a reaction within a transplanted organ or tissue involving both immunologic and non-immunologic responses that ultimately lead to damage or necrosis of some or all of the transplanted organ or tissue. An “organ” refers to a part of the body of a subject exercising a specific function (such as a heart, kidney, liver, or lung). A “tissue” refers to a collection of similar cell types (such as epithelium, connective, muscle and nerve tissue). A “transplanted tissue or organ” is meant to refer to a tissue or organ taken from one subject and implanted into a subject other than the subject from which the organ or tissue was taken.

“Suffering from a disease or condition” means that a subject is either presently subject to the signs and symptoms, or is more likely to develop such signs and symptoms than a normal subject in the population. Thus, methods of the present invention which relate to treatments of patients (e.g., methods for selecting a treatment, selecting a patient for a treatment, and methods of treating a disease or condition in a patient) can include primary treatments directed to a presently active disease or condition, secondary treatments which are intended to cause a biological effect relevant to a primary treatment, and prophylactic treatments intended to delay, reduce, or prevent the development of a disease or condition, as well as treatments intended to cause the development of a condition different from that which would have been likely to develop in the absence of the treatment.

“Treatment” refers to a process that is intended to produce a beneficial change in the condition of a mammal, e.g., a human, often referred to as a patient. A beneficial change can, e.g., include one or more of restoration of function, reduction of symptoms, limitation or retardation of progression of a disease, disorder, or condition or prevention, limitation or retardation of deterioration of a patient's condition, disease or disorder. In the context of targeted therapies, e.g., ADCC-based therapy, “treatment” or “treatable” is meant the ADCC-based therapy achieves a desired pharmacologic and/or physiologic effect on the disease or disorder. The effect may be prophylactic in terms of completely or partially preventing the disease/disorder or symptom thereof and/or may be therapeutic in terms of a partial or complete cure for the disease/disorder and/or adverse effect attributable to the disease/disorder. The terms include: (a) preventing the disease from occurring in a subject which may be predisposed to the disease but has not yet been diagnosed as having it; (b) inhibiting the disease, i.e., arresting its development; or (c) relieving the disease, i.e., causing remission or regression of the disease. The therapeutic agent may be administered before, during or after the onset of the disease or disorder. The treatment of ongoing disease, where the treatment stabilizes or reduces the undesirable clinical symptoms of the patient, is of particular interest. Such treatment is desirably performed prior to complete loss of function in the affected tissues.

“Target cell depletion assay” refers to a depletion assay, e.g., an ADCC assay measuring the reduction, depletion, or killing of cells targeted by an antibody. Target cell depletion assay can be done in vitro, e.g., human B cells used ex vivo with an anti-CD20 antibody and effector cells. In some embodiments, the target cell depletion assay can be in vivo, e.g., by measuring number of B cells in a subject after the administration of an anti-CD20 antibody by withdrawing blood samples, and measuring time-dependent depletion assays over a period of several weeks. Typically, MRD-FC is used to measure these populations. See Dass, et al. (2008) Arth. Rheum. 58:2993-2999; Vital, et al (2011) Arth. Rheum. 63:603-608; and Moreton, et al. (2005) J. Clin. Oncol. 23:2971-2979.

“Target cell repopulation assay” refers to a repopulation assay, e.g., an ADCC assay measuring the recovery (slow or fast) or recover rate of a target cell population following administration of an antibody, e.g., repopulation of B cells following administration of an anti-CD20 antibody. Assays can be in vivo, e.g., by measuring number of specific subsets of B cells repopulating in a subject after the administration of an anti-CD20 antibody by withdrawing blood samples, and measuring time-dependent depletion assays over a period of several weeks. Typically, MRD-FC is used to measure these populations in vitro. Faster repopulation is a measure of imminent disease relapse.

“Cell population targeted by an antibody” refers to a cell or group of cells that are specifically recognized by the antibody of interest, and in the context of ADCC, killed or lysed through an ADCC mechanism.

“Clinical trial” refers to an investigation of safety and efficacy of a treatment for a disease or disorder. Typically, clinical trials are carried out to obtain approval from a governmental regulatory agency for marketing a drug.

“Health service payer” refers to an entity that finances or pays for the medical treatment or therapy. A health service payer can include among others, an insurance company, a government entity, a private company, a PBM, an employer, a pension fund, or a patient.

### Theragnostics and Therapeutic Guidance

The term theragnostic (plural theragnostics) herein refers to products, tests, methods and procedures that can inherently guide treatment in (i) a single patient or (ii) a collection of patients, e.g., subset(s) of patients, entire disease-specific population covered by a payer or employer, suffering from a particular disease with a core objective of achieving excellent or near-excellent treatment outcomes in a reasonable timeframe. Such outcomes include disease remission, cure, excellent response, etc. Theragnostic procedures are inherently linked to therapies, treatments, and treatment guidance that collectively dictate efficacy and financial assurances, prior authorization, and the designing of a drug-specific formulary. Such assurances are products offered to payers and employers. Patients typically pay 20-30% of the specialty drug cost and hence, from the payment standpoint, patients are also payers.

Such theragnostic results are necessary for (a) prior authorization of a specialty drug mandating efficacy and financial assurances; (b) designing and developing a formulary, e.g., disease-specific drug formulary, such that the decision to include or not include a drug in the formulary is governed by the theragnostic results. Theragnostic products will guide in the selection of drugs, e.g., mechanism of action based treatment options in specific subsets of patients, with an objective of achieving remission or excellent response in defined subsets of patients. Some approved drugs with moderate or substandard efficacy profiles may be excluded entirely from the formulary. In a scenario where multiple molecules with the same MOA exist, e.g., biosimilars, a drug that is selected to the formulary based on such theragnostic products will have product differentiation and market enrichment advantages.

These theragnostic methods are mechanistic: (a) based on the mechanism of action of the drug itself and understanding why a patient or subset(s) of patients respond well given their particular genetic makeup (e.g., the primary therapeutic mechanism of rituximab monotherapy in B-NHL is ADCC); and (b) based on the pathophysiology of the disease itself as stratified, e.g., according to immunologically defined subtypes of disease (e.g., fibrinogen induced arthritis), disease severity, pharmacology, disease states, and physiology. The resolution of theragnostic procedures can be enhanced by combining more than one mechanistic determinants.

The core objectives of the use of theragnostics are: (i) to select an appropriate therapy for a given patient, given her disease characteristics, when multiple therapies are available to choose from; (ii) alternatively, to decide when not to select a particular therapy for a given patient, given her disease characteristics; (iii) to achieve clinical remission or excellent response when the patient is administered with a carefully chosen therapy, e.g., using a particular drug of choice at the first instance. Any or all of the above objectives can be accomplished by the use of theragnostic procedures.

Theragnostic functions entail: (a) therapeutic appropriateness, which is the selection of a therapeutic (drug), typically based on use of a particular drug, preferably a priori, when multiple therapeutics are available in a formulary to choose from, for a particular subset(s) of patients or an individual patient; (b) therapeutic guidance, which provides details of therapy, including aspects of specific drug dosing and schedule details during a treatment cycle; (c) therapeutic effectiveness, which is a measure of how well the therapy, including the drug, worked in that patient or how well the patient responded to that treatment during and at the end of the treatment cycle; and (d) selection of an alternate therapeutic (drug) that is considered as the next best choice based on, e.g., a mechanistic rationale, if the first choice failed to achieve reasonable therapeutic effectiveness. Any or all of the above objectives can be accomplished by the use of theragnostic procedures.

Furthermore, theragnostic procedures provide reliable, actionable treatment (and therapeutic) guidance for a single patient (what is generally referred to as precision or individualized medicine), subset(s) and subtype(s) of patients (stratified medicine), as well as for the entire disease population. Theragnostic methods provide significant advantages to patients (considered one of the payers in the specialty drug context), payers and employers in not only managing diseases and therapies, but also controlling costs both on a per patient basis and for the entire disease population being managed by a payer or employer. Other applications of theragnostics are in the areas of (a) providing therapeutic efficacy and financial assurances to payers, employers and patients; (b) selection of drug formularies as part of the prescription drug plans; (c) product differentiation from other commercially available drugs; and (d) market enrichment for a particular drug.

In a simpler embodiment, the theragnostic procedures can provide actionable treatment guidance by summary guidelines to achieve preferred outcomes. Thus, the guidance might be summarized by directing specific drug selection (from among alternatives; i.e., therapeutic appropriateness) for defined ranges of theragnostic readouts, directing specific therapy selection (from among alternatives of how drug is administered; i.e., therapeutic guidance) for defined ranges of theragnostic readouts, and directing overall therapy strategy (from among alternatives; i.e., therapeutic effectiveness) for defined ranges of theragnostic readouts, and specific exclusion criteria (from among alternatives; i.e., selection of alternative therapeutic) for particular other theragnostic readouts where treatment strategy is contraindicated (e.g., by toxicity or side effect) or first strategy fails. Thus, the guidelines may implicitly incorporate the theragnostic-guidance criteria with specific actionable directives based on theragnostic evaluations.

A single or a combination of DNA, RNA, protein, or immunological features may constitute a theragnostic product or evaluation. In addition, it may include metabolic evaluation, which may be useful for individualized pharmacology of half-life, absorption, distribution, metabolism, excretion, turnover, etc. Such examples include biomarkers, polymorphisms, gene expression profiles, protein expression profiles, presence or absence of specific protein markers or immunological, metabolic, physiological profiles, and many aspects which affect the therapy response. Furthermore, a single or a combination of companion diagnostic tests or in vitro diagnostic tests (e.g., theranostics, complementary diagnostics) may constitute a theragnostic procedure.

Currently available tests including biomarker tests (syn: complementary or companion diagnostic tests, theranostic tests etc. as defined elsewhere by others) have distinct insufficiencies in providing therapeutic and/or economic value. For example, specialty drugs in the checkpoint inhibitors class such as pembrolizumab (Merck), nivolumab (Bristol-Myers Squibb's), and atezolizumab (Roche's) block the interaction between the receptor programmed cell death protein 1 (PD1) on CD8⁺ T cells and its ligand (PDL1) on tumor cells. Whereas cancer cells co-opt this immune checkpoint pathway to limit T cell activity, the drugs remove this ‘brake’ and unleash the immune system on the cancer. Although responses to these therapies can be dramatic and durable in melanoma, only about one-third of patients respond. Response rates are significantly lower in non-small-cell lung cancer (NSCLC) and kidney cancer, at approximately 20-25% (Cancer Cell 27, 450-461; 2015).

Drug developers are consequently keen to identify biomarkers that can boost outcomes. While tumor PDL1 expression was an obvious first biomarker candidate, it has not lived up to expectations. PDL1 levels, as measured by immunohistochemistry (IHC), can identify groups of patients that are more likely to respond to PD1-PDL1 blockade, but it is not itself an absolute (e.g., reliable) marker: some patients with high PDL1 levels do not respond to treatment, and contrarily, a subset of those who test negative for PDL1 expression can derive considerable treatment benefit. This underscores the ambiguity around the use of biomarker(s) as to the insufficiency of providing real therapeutic or economic value.

Such ambiguities are also observed in other therapies, e.g., cetuximab (K-RAS mutations in metastatic colorectal cancer versus NSCLC), trastuzumab (Her-2 expression with a 3⁺ score in breast cancer).

Given this, regulatory approvals are restricted to the use of such a biomarker test for a particular therapy in a specific indication. PDL1 IHC is approved as a companion diagnostic only for pembrolizumab in NSCLC. Underscoring the ambiguity around the biomarker, FDA has approved it as a ‘complementary diagnostic’ in melanoma and for nivolumab in NSCLC, to assist but not dictate treatment decision-making. In part, the value of the biomarker may be limited by technical pitfalls such as irregular expression levels throughout the tumor and lack of a single, standardized IHC test. But a more fundamental limitation is that tumor expression of PDL1 does not provide the whole picture (Nature Rev. Cancer 16, 275-287; 2016).

The major insufficiencies of the currently available biomarker or diagnostic tests are: (a) they do not provide efficacy assurance (e.g., assured remission or excellent response); (b) they do not provide financial assurance; (c) not used for providing assurance-based payment (outcome) decisions; (d) not used for designing and developing a drug formulary by a prescription drug plan; (e) not used for providing disease-specific, population-wide therapy decisions (e.g., involving multiple therapies in a large patient population). Whereas theragnostic procedures delineated herein address these insufficiencies.

### Stratified Medicine, and Personalized Medicine

While specialty drugs (e.g., small molecule pharmaceuticals, protein biologics, therapeutic antibodies, etc.) are typically developed to interact with specific biological targets, populations generally show wide variations in response to the drug treatment, due in part, to genetic variations in populations, where the genetic variations affect therapeutic properties of the drug. These genetic variations can affect, among others, the direct biological target of the drug, metabolism of the drug, and/or the biological mechanisms by which the drug mediates its therapeutic effect. Thus, in some instances, a drug may only be effective in individuals or subset(s) of individuals, or subjects who have a particular genetic or protein variation and ineffective in those individuals who do have the particular genetic or protein variation, and may experience adverse side effects (e.g., increased toxicity). For example, a number of therapeutic antibodies have been developed for treating a variety of diseases including cancers, autoimmune diseases, and inflammatory disorders. However, it is generally acknowledged that many of these antibodies (e.g., rituximab in follicular lymphoma) work more effectively for some patients than others. Genetic variations can be determined at the DNA or RNA level whilst protein variations can be observed at the amino acid level.

In one embodiment, stratification of patients can be based on the therapeutic mechanism of action, e.g., ADCC. See US patent publication 20100291549 and WO 201309047820, both of which are incorporated herein by reference.

In another embodiment, stratification of patients can be based on disease severity mechanisms: (a) enhanced proinflammatory potential, and (b) impaired immune complex clearance. In another embodiment, multiple stratification mechanisms, e.g., ADCC and EPP mechanisms, or ADCC and ICC mechanisms, can be combined to develop a theragnostic strategy. See U.S. Provisional Patent Applications 62/332,315 and 62/322,325 both dated May 5, 2016, both of which are incorporated herein by reference.

In the area of antibody therapeutics, many antibodies have as its therapeutic mechanism, wholly or in part, antibody dependent cell-mediated cytotoxicity (ADCC). ADCC is a process of cell-mediated immunity in which effector cells of the immune system, such as natural killer (NK) cells, macrophages, neutrophils, and eosinophils, kill target cells that have been bound by specific antibodies. Destruction or killing of the target cell can occur through phagocytosis; ADDC-mediated lysis; ADCC-mediated apoptosis; and trogocytosis (antibody-dependent cytotoxicity mediated by polymorphonuclear granulocytes). The posited mechanism of ADCC is the binding of the effector cells to the Fc (constant) portion of the bound antibody through Fc receptors, particularly the Fcγ receptors, present on the effector cells. As such, variations or polymorphisms in the Fc receptor can affect the effectiveness of antibodies that work via the ADCC mechanism. The association between Fc receptor polymorphisms and ADCC has led to use of Fc genotypes for selecting patients for antibody-based therapies, e.g., US patent publication 20100291549 and WO 201309047820, both of which are incorporated herein by reference. While antibodies may have multiple mechanisms of action, e.g., ADCC, blocking cell signaling or neutralization, ADCC may be a major or contributory mechanism to the therapeutic effects. The contribution by other mechanisms does not preclude or obviate the ADCC mechanism.

“Enhanced proinflammatory potential” or “EPP” in the context of a disease or disorder characterized by enhanced proinflammatory potential refers to a process in which immune cells involved in inflammation, e.g., neutrophils, monocytes and macrophages, migrate, accumulate, and become activated at the sites of disease activity. In some embodiments, the disease or disorder characterized by enhanced proinflammatory potential is described as “AAI” or “attraction, accumulation, and activation of immune cells”. Generally, this mechanism leads to the localized accumulation of cytokines (e.g., TNF-α, IL-1, IL-1β, IL-6, GM-CSF, etc.), reactive oxidants, proteolytic enzymes which then collectively contribute to EPP. See U.S. Provisional Patent Application 62/322,325 dated May 5, 2016, which is incorporated herein by reference.

“Immune complex clearance” or “ICC” refers to clearance of immune complexes from a subject's body. The ICC mechanism is mediated by the interactions of IgG to Fcγ receptors. The clearance can be systemic or organ specific clearance. “Impaired ICC disease” or “impaired ICC disorder” refers to a disease or disorder characterized by abnormal or pathogenic levels of immune complexes, including immune complexes comprised of autoantibodies or microbial pathogens. See U.S. Provisional Patent Application 62/332,315 dated May 5, 2016, which is incorporated herein by reference.

While some patients achieve complete remission and some other patients achieve complete response, a majority of the patients achieve moderate and poor responses to specialty drugs of antibody class; the disease relapses in a significant majority of the patients. Relapse also occurs for certain number of patients following other types of specialty drugs, such as chemotherapies and small molecule drugs.

Treatment responsiveness can be predicted by measuring a therapeutic mechanism, e.g., the ADCC function or capacity of the patients, thus providing another determinative factor for selecting patients who are likely to have positive treatment outcomes, or conversely, excluding patients who are likely to have a negative treatment outcome, with the specialty drug treatment, e.g., with antibody maintenance therapy. Predicting responsiveness to antibody maintenance therapy, preferably a priori, can also allow selection of various treatment options, including alternatives to antibody therapy if the subject responds poorly to antibody maintenance therapy. This a priori identification and selection of patients who will respond (and not respond) to a therapy has significant commercial and therapeutic advantages, and will be useful to drug developers, theragnostic providers, physicians, health care payers, pharmacy benefit managers, disease and therapy management care specialists, and/or specialty pharmacists.

The reference stratification, also referred to as a reference index can be prepared for an ADCC treatable disease for a particular specialty drug. In some embodiments, the reference stratification can be prepared by determining the genotype of each subject in plurality of subjects having a disease or disorder treated with a specialty drug, and determining the treatment outcome or clinical outcome. The statistical significance of the linkage between the genotype and the responsiveness can be determined by standard statistical methods. The treatment outcome or clinical outcome assessments can use diagnostic measures known in the art and typically specific to each disease or disorder. See, e.g., World Health Organization International Classification of Diseases (ICD), e.g., ICD 10 and Merck Manual of Diagnosis and Therapy, Merck Publishing (2011). As further described in the present disclosure, the reference stratification data can be in printed form or stored in a computer memory. In some embodiments, the comparing of the determined genotype of the subject to the reference stratification can be implemented by a computer using methods standard in the art.

Accordingly, the terms “reference” and “control” as used herein refers to a standardized genotype to be used to interpret the genotype of a given patient and assign a prognostic class thereto. The reference or control may be a genotype that is obtained from a cell/tissue known to have the desired phenotype, e.g., responsive phenotype, and therefore may be a positive reference or control genotype. In addition, the reference/control genotype may be from a cell/tissue known to not have the desired phenotype, and therefore be a negative reference/control genotype.

In practicing methods, a subject or patient sample, e.g., cells or collections thereof, e.g., a blood sample or tissue or biopsy sample, is assayed to predict responsiveness of the patient to an antibody therapy, e.g., antibody maintenance therapy. For example, a patient with an ADCC-treatable disease who is responsive to antibody maintenance therapy will experience at least a slowing in disease progression; in some instances, at least a cessation of disease progression; in some instances, an improvement in health, i.e., a reversal of disease progression, a loss of disease symptoms, etc. In contrast, a patient with an ADCC-treatable disease who is not responsive to antibody maintenance therapy will not experience at least a slowing in disease progression, or at least a cessation in disease progression, or an improvement in health. In some embodiments in which the induction therapy comprises antibody therapy, responsiveness to an antibody maintenance therapy is responsiveness to maintenance therapy with the same antibody used in the induction therapy. In other embodiments in which the induction therapy comprises antibody therapy, responsiveness to an antibody maintenance therapy is responsiveness to maintenance therapy with an antibody other than that used in the induction therapy.

As further discussed, most any convenient metric available in the art may be used to measure and convey predictions of responsiveness to maintenance therapy. In some embodiments, predictions may be made in terms of progression free survival (PFS), overall survival (OS), relapse-free survival (RFS), and/or event-free survival (EFS), as the terms are defined herein and commonly used in the art, as further discussed below.

In some embodiments, the above-obtained information about the cell/tissue being assayed is employed to diagnose a host, subject or patient with respect to responsiveness to antibody maintenance therapy, as described above. In some embodiments, the above-obtained information is employed to give a refined probability prediction as to whether a subject will or will not respond to a particular specialty drug therapy and a financial payment decision based thereon.

In some embodiments, excellent responders may exhibit, e.g., at least about 85%, 90%, or higher mean or median response rates (or better than about 85 percentile measure of outcome among the unstratified population); very good responders may exhibit lesser measures of responsiveness, e.g., at least about 70%, 75%, or 80% response rates (or from about top 75th percentile to 85th of outcomes); good responders may have better than average response rates, e.g., at least about 55%, 60%, or 65% response rates (or from about top 55th percentile to 75th of outcomes); moderate responders will typically have near average response rates, e.g., in the range of about 45%, 50%, or 55% response rates (or from about 45th to 55th percentile of outcomes); below average responders may have lower response rates, e.g., below about 45%, 35%, or 30% (or from about 25th percentile to about 45th percentile of outcomes); very poor responders may have even lower response rates, e.g., below about 25%, 20%, or 15%, and non-responders may have even lower response rates, e.g., less than about 12%, 10%, or 5%.

In some embodiments, the average overall response rates to treatment for overall unstratified population will be in the 40% to 60% range. The above and below average responder subsets will preferably have at least about 7-15% better and lower relative mean or median responsiveness measures, respectively, and the good and poor responders will preferably have at least another 7-15% better and lower mean or median responsiveness measures, respectively. The very good and very poor responders will have correspondingly better and worse mean or median responsiveness measures, and the excellent and non-responders even more extreme. How many different stratification categories are used will depend largely upon the dispersion of the responsiveness measures across categories of treatment response, and the variation of individual responsiveness measures within each category of treatment response. In some embodiments, the range of responsiveness across the categories will range from less than about 10% to at least about 90%.

In some embodiments, the patients may be stratified by strata of percentile responsiveness ranges. Thus, the highest may be the top 15 percentile stratum of response, the next the second top 15 percentile stratum, etc., down to the lowest category of the bottom 15 percentile stratum, providing six strata of responsiveness. Improvement of responsiveness may be moving from one stratum to a higher stratum, preferably two or more.

In some embodiments, a reference stratification or reference index relating genotype group to categories of antibody maintenance treatment response can be used in both directions. It can be used to predict the responsiveness to maintenance treatment based on genotype at the relevant positions. This will be very useful for the patient and treating doctor, to provide means to arrive at likely response to alternative treatments. Conversely, for a given responsiveness to maintenance treatment, one can identify genotypes of patients which should achieve such response. Thus, a theragnostic provider or treatment payer may identify which patients are likely to response as indicated by the reference. Alternatively, for those who respond poorly, additional or alternative treatment strategies may be applied. In other embodiments, those who would respond poorly are not treated with an available treatment with low efficacy for those patients. Thus, financial decisions may be based upon such projections or predictions. For a payer, particular treatment strategies might be paid only for patients whose genotypes indicate good responsiveness. Alternatively, those patients whose responsiveness is low may be directed immediately to alternative treatment strategies which have higher success rates. Where the maintenance treatment may improve response after a period of maintenance therapy for certain genotype groups, recognizing which patients will respond can provide many benefits, both to the payer and to the patient.

In the embodiments herein, the subject, preferably a human subject, has had or will have a specialty drug as an induction therapy. In some embodiments, the induction therapy can comprise chemotherapy. In some embodiments the induction therapy can comprise antibody therapy. In some embodiments, the subject has previously received or is receiving antibody maintenance for the disease or disorder.

As will be apparent to the skilled artisan in view of the teaching provided herein and given the general conservation of the Fc portion of antibodies, many therapeutic antibodies, particularly those with IgG isotypes, are expected to be influenced similarly by polymorphisms at FcγRIIA and/or FcγRIIIA, including the polymorphisms at amino acid position 131 of FcγRIIA and amino acid position 158 of FcγRIIIA. Accordingly, the genotype and the predicted responsiveness can be applied to many antibodies, particularly where the Fc region is human IgG₁, that have ADCC as a therapeutic mechanism across many different diseases and disorders, and therefore applicable to the various methods described in the present disclosure. U.S. Pat. No. 8,592,149, US patent publication 20100291549, and WO 201309047820, all of which are incorporated herein by reference; additionally, U.S. Provisional Patent Applications 62/332,315 and 62/322,325 both dated May 5, 2016, both of which are incorporated herein by reference.

In some embodiments, while genotype evaluation results may be reported separately from therapy recommendations, the interpretation of genotype results will often be provided in a report describing preferred or standard treatment options. Thus, for the various methods of the present disclosure, the genotype information, the stratification, the selection/exclusion of subjects for therapy, the predicted treatment outcome, and the treatment options, as further discussed in the present disclosure can be reported in electronic, web-based, or paper form to the human subject, a health care payer, third party payer, a health care provider, a specialty pharmacist, a DTM care provider, a physician, a pharmacy benefits manager, or a government office. Insurance coverage or financial obligations may then be based thereon.

### Selecting Patients

As described above, the methods for predicting responsive can be applied to the selection of subjects who are likely to respond positively to specialty drugs. Conversely identification of subjects who respond poorly provides an opportunity to choose alternative treatments that could produce better treatment outcomes than the said specialty drug. In addition to the benefit for the patient, the ability to select subjects who are likely to have a more favorable treatment outcome provides many advantages to payers, providers, theragnostic providers, DTM care providers, specialty pharmacists, and insurers.

In another embodiment, if the specialty drug is an ADCC-mediated antibody therapy, the method of treating can further comprise measuring the level of functional capacity of immune cells, e.g., immune effector cells, specifically, ADCC capacity or function in the subject, thus providing another independent criterion or metric for selecting subjects who will likely have a positive treatment outcome for the antibody maintenance therapy. Examples include selective or non-selective depletion of specific subsets of B-cells, inflammatory macrophages, tumor infiltrating macrophages, inflammatory NKT-cells, etc. Selective repopulation of specific subsets of B-cells is yet another example of measurement of ADCC function. U.S. Pat. No. 8,592,149, US patent publication 20100291549, and WO 201309047820, each of which is incorporated herein by reference; additionally, U.S. Provisional Patent Applications 62/332,315 and 62/322,325 dated May 5, 2016, both of which are incorporated herein by reference.

### Treating Patients with Specialty Drugs

In some embodiments, the present disclosure further provides methods of treating subjects with a specialty drug, e.g., an ADCC treatable disease or disorder based on selection of a subject who is likely to have positive treatment outcomes. In some embodiments, a method of treating a human subject having an antibody dependent cell-mediated cytotoxicity (ADCC)-treatable disease or disorder with an antibody maintenance therapy comprises:

determining a genotype of the subject for one or more Fcγ receptor functional polymorphisms affecting ADCC activity, wherein the Fcγ receptor functional polymorphism is selected from a FcγRIIa polymorphism and a FcγRIIIA polymorphism;

stratifying the human subject into a responsiveness group based on the determined genotype, and selecting or excluding the human subject for antibody maintenance therapy based on the stratification; and

administering to the selected human subject the antibody maintenance therapy regimen.

In some embodiments, a method of treating a subject having an antibody dependent cell-mediated cytotoxicity (ADCC)-treatable disease or disorder with an antibody maintenance therapy comprises:

selecting or excluding the human subject for antibody maintenance therapy by stratifying the human subject into a responsiveness group based on a determined genotype of the human subject for one or more Fcγ receptor functional polymorphisms affecting ADCC activity, wherein the Fcγ receptor polymorphism is selected from a FcγRIIa functional polymorphism and a FcγRIIIA functional polymorphism; and

administering to the selected human subject the antibody maintenance therapy regimen.

As described herein, stratification of the subject into a responsiveness group is carried out by comparing the determined genotype of the human subject to a reference stratification that relates responsiveness to antibody maintenance therapy for the ADCC treatable disease to genotypes of the Fcγ receptor polymorphism.

As further described below, the method of treating can further comprise measuring the level of ADCC capacity or function in the subject, providing another independent criterion or metric for treating subjects who will likely have a positive treatment outcome for the antibody maintenance therapy. Examples include selective or non-selective depletion of specific subsets of B-cells, inflammatory macrophages, tumor infiltrating macrophages, inflammatory NKT-cells, etc. Selective repopulation of specific subsets of B-cells is yet another example of measurement of ADCC function as well as disease remission and relapse patterns. Responsiveness predictions may be a component of an insurance coverage decision.

### Diseases Treated with Specialty Drugs

In the methods herein, a wide variety of types of diseases and disorders can be treated with specialty drugs. In some embodiments, the treatable disease or disorder is selected from a neoplastic disease, an autoimmune disease, an inflammatory disorder, a microbial infection, or allograft rejection.

In some embodiments, the disease treated with a specialty drug comprises a neoplastic disease, i.e., hyperproliferative disorders, or malignancies, which are characterized by unregulated cell growth. Neoplastic diseases include, among others, acute lymphoblastic leukemia (ALL); acute myeloid leukemia (AML); bladder cancer; bone cancer; bowel cancer; brain tumors; breast cancer; cancer of unknown primary; carcinoid; cervical cancer; choriocarcinoma; chronic lymphocytic leukemia (CLL); chronic myeloid leukemia (CML); colon cancer; colorectal cancer; endometrial cancer; eye cancer; gallbladder cancer; gastric cancer; gestational trophoblastic tumors (GTT); hairy cell leukemia; head and neck cancer; Hodgkin's lymphoma; kidney cancer; laryngeal cancer; leukemia; liver cancer; lung cancer; non-small cell lung cancer; lymphoma; melanoma skin cancer; molar pregnancy; mouth and oropharyngeal cancer; myeloma; nasal and sinus cancers; nasopharyngeal cancer; B-cell non-Hodgkin's lymphoma (B-NHL); neuroblastoma; esophageal cancer; ovarian cancer; pancreatic cancer; penile cancer; prostate cancer; rectal cancer; salivary gland cancer; skin cancer (non-melanoma); soft tissue sarcoma; stomach cancer; testicular cancer; thyroid cancer; unknown primary cancer; uterine cancer; vaginal cancer; vulval cancer; and the like.

In some embodiments, one class of neoplastic diseases for which a number of ADCC-based therapies have been developed is the hematological malignancies, e.g., B-cell malignancies, including non-Hodgkin's Lymphomas (B-NHL). B-cell malignancies are those disorders that derive from cells in the B cell lineage, typically including hematopoietic progenitor cells expressing B lineage markers, pro-B cells, pre-B cells, B-cells and memory B cells; and that express markers typically found on such B lineage cells. The B-NHL are a variety of B-cell neoplasms, and include precursor B-lymphoblastic leukemia/lymphoma; peripheral B-cell neoplasms, e.g., B-cell chronic lymphocytic leukemia; prolymphocytic leukemia; small lymphocytic lymphoma; mantle cell lymphoma; follicular lymphoma; marginal zone B-cell lymphoma; splenic marginal zone lymphoma; hairy cell leukemia; diffuse large B-cell lymphoma; T-cell rich B-cell lymphoma, Burkitt's lymphoma; high-grade B-cell lymphoma, (Burkitt-like); etc. Markers that are specifically found on B cells that may be used as target antigens for ADCC-based therapies include CD45R, which is an exon-specific epitope found on essentially all B cells, and is maintained throughout B cell development (Coffman, et al. (1982) Immunol. Rev. 69:5-23); CD19, CD20, CD22, and CD23, which are selectively expressed on B cells and have been associated with B cell malignancies (Kalil and Cheson (2000) Drugs Aging 16:9-27; U.S. Pat. No. 6,183,744, herein incorporated by reference); surface immunoglobulin, including epitopes present on the constant regions or idiotypic determinants, which have been utilized in immunotherapy (Caspar, et al. (1997) Blood 90:3699-706); and the MB-1 antigen, found on all normal immunoglobulin (Ig)-expressing cells, but not on T cells, thymocytes, granulocytes, or platelets, and expressed by virtually all Ig-expressing B cell tumors (Link, et al. (1986) J. Immunol. 137:3013-8). Other B cell antigens of interest known to be expressed, e.g., on B non-Hodgkin's lymphomas, are Muc-1; B5; BB1; and T9 (Freedman, et al. (1987) Leukemia 1:9-15). Of particular interest is the CD20 antigen, a human B cell marker that is expressed during early pre-B cell development and remains until plasma cell differentiation. U.S. Pat. No. 5,736,137, herein incorporated by reference, describes the chimeric antibody “C2B8” (also known as RITUXAN®, rituximab, MABTHERA®) that binds the CD20 antigen and its use to treat B cell lymphomas.

ADCC-based therapies have also been developed for solid tumors, e.g., colorectal cancer, non-small cell lung cancer, small cell lung cancer, ovarian cancer, breast cancer, head and neck cancer, renal cell carcinoma, and the like. The exemplary antigens include-CD52, VEGF, CD30, EGFR, CD22, CD33, CD20, CTLA4, CD2, CD25, EphA2, G25, ErbB2, phosphatidyl serine, and HER2.

In some embodiments, the disease or disorder treated with a specialty drug is an autoimmune disease. Autoimmune diseases are diseases characterized by an overactive immune response of the body against substances and tissues normally present in the body. Examples of autoimmune diseases include, among others, agammaglobulinemia, amyotrophic lateral sclerosis, ankylosing Spondylitis, autoimmune cardiomyopathy, autoimmune hemolytic anemia, autoimmune lymphoproliferative syndrome, autoimmune peripheral neuropathy, autoimmune pancreatitis, autoimmune uveitis, Behçet's disease, Berger's disease, celiac disease, Chagas disease, chronic obstructive pulmonary disease, Churg-Strauss syndrome, Crohn's disease, colitis, diabetes mellitus type 1, discoid lupus erythematosus, Goodpasture's syndrome, Graves' disease, Guillain-Barré syndrome (GBS), idiopathic pulmonary fibrosis, idiopathic thrombocytopenic purpura, IgA nephropathy, inclusion body myositis, chronic inflammatory demyelinating polyneuropathy, Kawasaki's disease, mixed connective tissue disease, multiple sclerosis, myasthenia gravis, hemolytic disease of the newborn (HDN), pemphigus vulgaris, polymyositis, progressive inflammatory neuropathy, psoriasis, psoriatic arthritis, rheumatoid arthritis (RA), juvenile RA, Sjögren's syndrome, systemic lupus erythematosus, lupus nephritis, systemic vasculitides, and Wegener's granulomatosis, B-cell lymphoproliferative disorders and malignancies due to HCV infection of the B-cells, and the like.

In some embodiments, the disease treatable with specialty drug is an inflammatory disease. In many instances, inflammatory diseases or disorders occur in the context of autoimmune diseases. Exemplary inflammatory diseases include, among others, Crohn's disease, ulcerative colitis, inflammatory bowel disease, ileitis and enteritis; vaginitis; psoriasis and inflammatory dermatoses such as dermatitis, eczema, atopic dermatitis, allergic contact dermatitis, urticaria; vasculitis; spondyloarthropathies; scleroderma; respiratory allergic diseases such as asthma, allergic rhinitis, hypersensitivity lung diseases, osteoarthritis, multiple sclerosis, systemic lupus erythematosus, diabetes mellitus, glomerulonephritis, and the like.

In some embodiments, the disease or disorder treated with a specialty drug is a microbial infection by a pathogen, including viruses, bacteria, fungi, protozoa, and multicellular parasites. Microbial infections of interest include hepatitis C virus, HIV, malaria, and tuberculosis.

In some embodiments, the disease or disorder treated with a specialty drug is an allograft, i.e. transplant, rejection. Organs that are typically transplanted include heart, kidneys, liver, lungs, pancreas, intestine, and thymus. Antibodies used to treat organ rejection can be targeted to markers expressed on cells that mediate allograft rejection, e.g., CD25 (anti-CD25) and CD3 (anti-CD3).

### Therapeutic Antibodies

An antibody for any of the methods in the present disclosure, e.g., predicting responsiveness, selection, treatment, theragnostic applications, payment decisions, etc., is used in the broadest sense, as defined herein, so long as they exhibit the desired biological activity (e.g., binding to target and mediating ADCC). Antibodies for the purposes herein include, among others, chimeric, humanized or fully human antibodies. In some aspects, a combination of one or more antibodies with different specificities, either for epitopes of a single antigen, or for multiple antigens, may be used.

The appropriate antibody can be chosen by the skilled artisan in view of the treatable disease or condition and the target of the antibody. For example, in some embodiments, where the ADCC treatable disease is a neoplastic disease, the antibodies can comprise an anti-CD19 antibody, anti-CD20 antibody, anti-CD22, anti-CD25 antibody, anti-CD30 antibody, anti-CD33 antibody, anti-CD52 antibody, anti-EGFR, anti-EphA2 antibody, anti-GD2 antibody, anti-G250 antibody, anti-ErB2 antibody, anti-folate receptor a antibody, anti-folate receptor antibody, or anti-phosphatidylserine antibody, or combinations thereof, depending on the specific neoplastic disease.

For the specific neoplastic disease of B non-Hodgkin's lymphoma (NHL), the antibody can comprise anti-CD20 antibody. Exemplary anti-CD20 antibodies can be selected from, among others, rituximab, ofatumumab, ibritumomab, tositumomab, veltuzumab, and obinutuzumab. A biosimilar or biosuperior anti-CD20 antibody can also be selected instead.

In some embodiments, where the treatable disorder is an autoimmune disease, the antibody for maintenance therapy can comprise, among others, an anti-CD29 or anti-CD20 antibody. Exemplary anti-CD20 antibodies that can be used to treat autoimmune diseases include those described for neoplastic diseases above. For the autoimmune disease systemic lupus erythematosus, an anti-CD20 antibody, such as rituximab or veltuzumab can be selected. Exemplary antibodies or antibody fusion therapies that can be used to treat autoimmune diseases include infliximab, etanercept, adalimumab, rituximab, certolizumab pegol, golimumab, tocilizumab, abatacept, etc.

In some embodiments, various methods can be used to assess whether a specialty drug exerts a specific therapeutic mechanism in a specific indication, e.g., an antibody has a therapeutic mechanism involving ADCC. In some embodiments, in vitro or ex vivo ADCC assays can be employed, with effector cells from healthy subjects or from subjects suffering from an ADCC treatable disease, e.g., B-NHL. In the latter case, the ADCC activity can be compared between high responders, e.g., genotype group I, H/H¹³¹ FcγRIIA and V/V¹⁵⁸ FcγRIIIA, and low responders, e.g., genotype group IX, R/R¹³¹ for FcγRIIA and F/F¹⁵⁸ for FcγRIIIA, where a significant difference in ADDC activity would implicate an ADCC-based therapeutic mechanism. Alternatively, the association or linkage of Fcγ polymorphisms that affect ADCC (e.g., FcγRIIA and FcγRIIIA polymorphisms) and responsiveness to antibody therapy can also be a basis for ascertaining ADCC activity. See U.S. Pat. No. 8,592,149, US patent publication 20100291549, and WO 201309047820, each of which are incorporated herein by reference; additionally, U.S. Provisional Patent Applications 62/332,315 and 62/322,325 dated May 5, 2016, both of which are incorporated herein by reference.

### Treatment Responsiveness

In practicing methods of therapeutic efficacy assurance, a subject or patient sample, e.g., cells or collections thereof, e.g., a blood sample or tissue sample, is evaluated to predict responsiveness of the patient to a specialty drug therapy, e.g., an antibody maintenance therapy. For example, a patient with an ADCC-treatable disease who is responsive to antibody maintenance therapy will experience at least a slowing in disease progression; in some instances, at least a cessation of disease progression; in some instances, an improvement in health, i.e., a reversal of disease progression, a loss of disease symptoms, etc. In contrast, a patient with an ADCC-treatable disease that is not responsive to antibody maintenance therapy will not experience at least a slowing in disease progression, or at least a cessation in disease progression, or an improvement in health. In some embodiments in which the induction therapy comprises antibody therapy, responsiveness to an antibody maintenance therapy is responsiveness to maintenance therapy with the same antibody used in the induction therapy. In some embodiments in which the induction therapy comprises antibody therapy, responsiveness to an antibody maintenance therapy is responsiveness to maintenance therapy with an antibody other than that used in the induction therapy.

It is to be understood that the evaluations for responsiveness will depend on the specific disorder and the standards and methods applied for that disorder. For example, diagnosis and evaluation of cancer treatment are described in, among others, DeVita, et al. (eds. 2011) Cancer: Principles and Practice of Oncology (9th Ed.) Lippincott Williams and Wilkins; Cohen, et al. (2010) Infectious Diseases (3d ed.); and Beers, et al. (eds. 2011) Merck Manual of Diagnosis and Therapy, Merck Publishing. Diagnosis and evaluation of autoimmune diseases are described in, among others, Brenner (ed. 2011) Autoimmune Diseases: Symptoms, Diagnosis and Treatment Nova Science Pub. Diagnosis and evaluation of infectious diseases are described, in among others, Mandel, et al. (eds. 2009) Mandell: Principles and Practice of Infectious Diseases: Expert Consult Premium Edition (7th ed.) Churchill Livingstone Elsevier; and Schlossberg (ed. 2008) Clinical Infectious Disease Cambridge Univ. Pr. Diagnosis and evaluation of allograft rejection are described in, among others, Kobayashi and Arai (eds. 2012) Acute Rejection: Risk Factors, Management and Complications Nova Science Publishers; and Russell and Cohn (eds. 2012) Transplant Rejection Book on Demand Ltd.

In some embodiments, such as neoplasms, following obtainment of the genotype from the sample being assayed, the genotype is evaluated to determine whether the subject/host/patient is responsive to the anti-neoplastic therapy of interest. In some embodiments, the obtained genotype may be compared with a reference or control to make a diagnosis regarding the therapy responsive phenotype of the cell or tissue, and therefore host, from which the sample was obtained/derived. The terms “reference” and “control” as used herein mean a standardized genotype to be used to interpret the genotype of a given patient and assign a prognostic class thereto. The reference or control may be a genotype that is obtained from a cell/tissue known to have the desired phenotype, e.g., responsive phenotype, and therefore may be a positive reference or control genotype. In addition, the reference/control genotype may be from a cell/tissue known to not have the desired phenotype, and therefore be a negative reference/control genotype.

In the embodiments herein, any convenient metric may be used to measure and convey predictions of responsiveness to maintenance therapy. For example for oncology indications, responsiveness and associated predictions may be made in terms of remission, progression free survival (PFS), overall survival (OS), relapse-free survival (RFS), time to progression (TTP), and/or event-free survival (EFS) as defined herein and as practiced in the art. Evaluation of target lesions include Complete Response (CR), which is disappearance of all target lesions; Partial Response (PR), which is at least a 30% decrease in the sum of the Longest Diameter (LD) of target lesions, taking as reference the baseline sum LD; Stable Disease (SD), which is neither sufficient shrinkage to qualify for PR nor sufficient increase to qualify for PD, taking as reference the smallest sum LD since the treatment started; or Progressive Disease (PD), which is at least a 20% increase in the sum of the LD of target lesions, taking as reference the smallest sum LD recorded since the treatment started or the appearance of one or more new lesions.

Also in oncology indications, evaluation of target lesions include Complete Response (CR), which is disappearance of all target lesions; Partial Response (PR), which is at least a 30% decrease in the sum of the Longest Diameter (LD) of target lesions, taking as reference the baseline sum LD; Stable Disease (SD), which is neither sufficient shrinkage to qualify for PR nor sufficient increase to qualify for PD, taking as reference the smallest sum LD since the treatment started; or Progressive Disease (PD), which is at least a 20% increase in the sum of the LD of target lesions, taking as reference the smallest sum LD recorded since the treatment started or the appearance of one or more new lesions.

A specific example is demonstrated by definition of end points for follicular lymphoma. Complete response (CR) required the resolution of all symptoms and signs of lymphoma including bone marrow clearing, for at least 28 days. Partial response (PR) required a 50% decrease in the sum of the products of perpendicular measurements of lesions, without any evidence of progressive disease for at least 28 days. Patients who did not achieve a CR or PR were considered non-responders (NR), even if there was a net decrease (<50%) of measurable disease. Time to progression was measured from the first infusion until progression. See, e.g., McLaughlin, et al. (1998) J. Clin. Oncol. 16:2885; Cheson, et al. (1999) J. Clin. Oncol. 17:1244; and Weng and Levy (2003) J. Clin. Oncol. 21:3940.

For autoimmune disorders, different measures of responsiveness or lack thereof to treatment exist among the different autoimmune diseases. Since most are progressive and chronic, they may have similar staging and episodic conditions as found in many oncology conditions. See the American College of Rheumatology (ACR) website www.rheumatology.org. ACR scores represent the percentage of reduction (20%, 50%, 70%) in tender and swollen joint counts, in addition to a corresponding improvement in three of the following five parameters: acute phase reactant (such as erythrocyte sedimentation rate), Patients Global Assessment of Disease Activity, Physicians Global Assessment of Disease Activity, Pain scale, and Health Assessment Questionnaire (HAQ). DAS28 is a measure of disease activity in RA. The score is calculated by a complex mathematical formula, which includes the number of tender and swollen joints (out of a total of 28), the erythrocyte sedimentation rate (a marker of systemic inflammation), and the patient's ‘global assessment of global health’ (indicated by marking a 10 cm line between ‘very good’ and ‘very bad’). A DAS28 score greater than 5.1 indicates severe active disease, less than 3.2 suggests low disease activity, and less than 2.6 is considered DAS remission.

In lupus (SLE), there are two major scoring systems to evaluate the activity of lupus in clinical studies: SLE Disease Activity Index (SLEDAI) and British Isles Lupus Activity Group (BILAG). SLEDAI is a list of 24 items, 16 of which are clinical items such as seizure, psychosis, organic brain syndrome, arthritis, blood vessel inflammation, etc. The other criteria are laboratory results such as urinalysis testing, blood complement levels, increased anti-DNA antibody levels, low platelets, and low white blood cell count. These items are scored based on whether these manifestations are present or absent in the previous 10 days. Organ involvement is weighted. An improvement of this, the SELENA-SLEDAI adds some clarity to some of the definitions of activity in the individual items, but does not change the basic scoring system. BILAG is an organ-specific 86-question assessment based on the principle of the doctor's intent to treat, which requires an assessment of improved (1), the same (2), worse (3), or new (4) over the last month. For screening, treatment, and management of lupus and lupus nephritis, ACR guidelines are adopted (ACR Ad Hoc Committee on SLE Guidelines (1999) Arth. Rheum 42:1785-96; Hahn, et al. (2012) Arth. Care & Res. 64:797-808).

Similar scoring methods have been devised for other immune disorders, e.g., in Sjögren's syndrome, polymyalgia rheumatica, osteoarthritis, multiple sclerosis, systemic vasculitides, Wegener's granulomatosis, and others. See, e.g., Beers, et al. (eds. 2011) The Merck Index of Medical Information; American Autoimmune Related Diseases Association, Inc. (www.aarda.org), www.rheumatology.org, and websites of particular diseases.

### Patient Therapeutic Adherence or Compliance

The medical and pharmacy industries classify the term patient medication adherence (or compliance) into three features: patient initiation adherence, which is the initiation of the pharmacotherapy by patient; patient persistence, which is defined as the length of time a patient fills his/her prescriptions; and patient execution adherence, which is the comparison between the prescribed drug-dosing regimen and the real patient's drug-taking behavior. These are applicable to individual patients, but the terms can also be applied to groups of patients or an entire disease population such as rheumatoid arthritis, multiple sclerosis, etc., referring to various levels of patient therapeutic adherence.

Despite advances in the effectiveness of therapies to manage certain diseases, and improvement in medication regimens to simplify administration, patient therapeutic adherence remains a significant issue. By some estimates the lack of patient therapeutic adherence, and therefore, compromised treatment effectiveness increase healthcare costs by over $100 billion in the US alone. Among chronic medical conditions approximately 50% of all patients are medication non-adherent.

A variety of factors contribute to patient non-adherence including: lack of medication efficacy, medication side effects or toxicities, complexity of the medication regimen, patient education and socioeconomic level, patient-healthcare provider relationship, patient understanding of the medication and disease, patient perception of medication efficacy, language barriers, prescription cost, and affordability. In the U.S healthcare system, nearly 20-30% of the specialty drugs cost is borne by patients by way of co-insurance.

Non-adherence to these medications, particularly high-priced specialty drugs, causes a substantial economic burden to the healthcare system by way of wasted drug costs and the costs associated with poor patient outcomes in often difficult-to-treat chronic diseases and cancers.

Rheumatoid arthritis (RA) is an illustrative example of the problem. Biologic disease modifying anti-rheumatic drugs (DMARDs) on average cost over $3,000 per month, and these agents are effective in reducing disease activity and radiological progression and can improve long-term functional outcomes in patients. Non-adherence can lead to disease flares and increased disability, yet patient therapeutic adherence rates in people with RA are low. Literature reviews and reports from large pharmacy benefit managers indicate biologic DMARD medication non-adherence is in the 40-45% range (Duffant, et al. (2014) Succeeding in the Rapidly Changing U.S. Specialty Market IMS Health). In longitudinal studies in RA population, 12-24% of the patients are consistently non-adherent, whereas only 30-35% of the patients are consistently adherent (see van den Bemt, et al. (2012) Expert Rev. Clin. Immunol. 8:337; van Dulmen, et al. (2008) BMC Health Serv. Res. 27:47).

Contributing to RA biologic DMARD medication non-adherence is the “trial and error” approach to treating the disease. Approximately >30-60% of the patients treated with some of the biologic DMARDS, e.g., anti-TNF-alpha therapies, are non-responders or poor responders. Physicians do not have adequate tools to identify the most appropriate biologic DMARD for each patient and hence up to 50% of patients receiving these agents will discontinue therapy because of lack of efficacy and or side effects. Analyses derived from large pharmacy and medical claims datasets indicate that the patient therapeutic adherence rates exceed 80% when a biologic DMARD is therapeutically effective (Duffant, et al. (2014) Succeeding in the Rapidly Changing U.S. Specialty Market IMS Health).

A method or system to identify the most appropriate biologic DMARD at therapy initiation and during treatment would, on average, markedly improve both the patient therapeutic adherence rate for these medications and treatment outcomes, and thus significantly lower direct medical costs, and reduce the costs associated with inappropriate drug usage by hundreds of millions of dollars.

In one embodiment, novel methods to improve therapeutic outcomes could therefore significantly improve patient therapeutic adherence of specialty drugs. In one instance, theragnostic evaluation procedures are used a priori to identify and administer the right specialty drug in a given patient such that remission or excellent response can be achieved, which leads to better patient therapeutic adherence in that patient, preferably 60-80%. In some instances, 80-90% patient therapeutic adherence is achieved; and in yet other instances, 90-100% patient therapeutic adherence is achieved. Improved therapeutic response results in decreased current and future treatment costs.

In another embodiment, novel methods of providing therapeutic efficacy and (or) financial assurances can improve patient therapeutic adherence of specialty drugs. In one instance, such assurances are guided by theragnostic-evaluation procedures. In one embodiment, providing therapeutic efficacy assurance or financial assurance leads to better patient therapeutic adherence in a patient, preferably 60-80%. For example, in a treatment cycle consisting of 10 weekly injections, if a patient fails to take 4 injections in a timely manner, then the patient therapeutic adherence rate is 60%. In some instances, 80-90% patient therapeutic adherence is achieved; and in yet other instances, 90-100% patient therapeutic adherence is achieved. In another embodiment, providing financial assurance leads to better patient therapeutic adherence in a patient, preferably 60-80%. In some instances, 80-90% patient therapeutic adherence is achieved; and in yet other instances, 90-100% patient therapeutic adherence is achieved.

### Genetic, Proteomic Markers, Polymorphism Determination

In the embodiments herein, many convenient protocols for assaying a sample for the above one or more target polymorphisms may be employed in the subject methods. In some embodiments, the target polymorphism will be detected at the protein level, e.g., by assaying for a polymorphic protein. In some embodiments, the target polymorphism can be detected at the nucleic acid level, e.g., by assaying for the presence of nucleic acid polymorphism, e.g., a single nucleotide polymorphism (SNP) that causes expression of the polymorphic protein. In one instance, e.g., Fcγ receptor polymorphism can be determined by various methods known in the art. Generally, a sample is obtained from an individual with an ADCC-treatable disease, the sample is assayed to determine the genotype of the individual from which the sample was obtained with respect to at least one, i.e., one or more, polymorphisms in the FcγRIIA gene and/or at least one, i.e., one or more polymorphisms in the FcγRIIIA gene. Nucleic acid sequencing or analytical methods will often be used.

In some embodiments, polynucleotide samples derived from (e.g., obtained from) an individual may be employed. A biological sample that comprises a polynucleotide from the individual is suitable for use in the methods of the invention. The biological sample may be processed to isolate the polynucleotide. Alternatively, whole cells or other biological samples may be used without isolation of the polynucleotides contained therein. Detection of a target polymorphism in a polynucleotide sample derived from an individual can be accomplished by means well known in the art, including, but not limited to, amplification of a sequence with specific primers; determination of the nucleotide sequence of the polynucleotide sample; hybridization analysis; single strand conformational polymorphism analysis; denaturing gradient gel electrophoresis; mismatch cleavage detection; and the like. Detection of a target polymorphism can also be accomplished by detecting an alteration in the level of a mRNA transcript of the gene; aberrant modification of the corresponding gene; the presence of a non-wild-type splicing pattern of the corresponding mRNA; an alteration in the expression level of the corresponding polypeptide; and/or an alteration in corresponding polypeptide activity. Detailed description of these techniques can be found in a variety of publications, including, e.g., Taylor (ed. 1997) Laboratory Methods for the Detection of Mutations and Polymorphisms in DNA CRC Press, and references cited therein. In some embodiments, genomic DNA or mRNA can be used directly. Alternatively, the region of interest can be cloned into a suitable vector and grown in sufficient quantity for analysis. The nucleic acid may be amplified by conventional techniques, such as a polymerase chain reaction (PCR), to provide sufficient amounts for analysis. See, e.g., Bartlett and Stirling (eds. 2000) PCR Protocols in Methods in Molecular Biology, Humana Press; and Innis, et al. (eds. 1999) PCR Applications: Protocols for Functional Genomics Academic Press. Once the region comprising a target polymorphism has been amplified, the target polymorphism can be detected in the PCR product by nucleotide sequencing, by SSCP analysis, or any other methods known in the art. PCR may also be used to determine whether a polymorphism is present by using a primer that is specific for the polymorphism. Parameters such as hybridization conditions, polymorphic primer length, and position of the polymorphism within the polymorphic primer may be chosen such that hybridization will not occur unless a polymorphism present in the primer(s) is also present in the sample nucleic acid. Those of ordinary skill in the art are aware of how to select and vary such parameters. See, e.g., Saiki, et al. (1986) Nature 324:163-66; and Saiki, et al. (1989) Proc. Natl. Acad. Sci. USA 86:6230-34. Exemplary methods for determining FcγRIIA and FcγRIIIA polymorphisms are described in Delgado, et al. (2010) Cancer Res. 70:9554-61. Direct sequencing methods may also be used.

In some embodiments, oligonucleotide ligation can be used to detect polymorphisms. See, e.g., Riley, et al. (1990) Nucleic Acids Res. 18:2887-2890; and Delahunty, et al. (1996) Am. J. Hum. Genet. 58:1239-1246. In some embodiments, hybridization with the variant sequence may also be used to determine the presence of a target polymorphism. Hybridization analysis can be carried out in a number of different ways, including, but not limited to Southern blots, Northern blots, dot blots, microarrays, etc. The hybridization pattern of a control and variant sequence to an array of oligonucleotide probes immobilized on a solid support, as described in U.S. Pat. No. 5,445,934, or in WO 95/35505, may also be used as a means of detecting the presence of variant sequences. Identification of a polymorphism in a nucleic acid sample can be performed by hybridizing a sample and control nucleic acids to high density arrays containing hundreds or thousands of oligonucleotide probes. See, e.g., Cronin, et al. (1996) Human Mutation 7:244-255; and Kozal, et al. (1996) Nature Med. 2:753-759.

In some embodiments, the genotype is determined by assaying the polymorphic protein. Detection may utilize staining of cells or histological sections with labeled antibodies, performed in accordance with conventional methods. Cells are permeabilized to stain cytoplasmic molecules. The antibodies of interest are added to the cell sample, and incubated for a period of time sufficient to allow binding to the epitope. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Alternatively, the secondary antibody conjugated to a fluorescent compound, e.g., fluorescein, rhodamine, Texas red, etc. Final detection uses a substrate that undergoes a color change in the presence of the peroxidase. The absence or presence of antibody binding may be determined by various methods, including flow cytometry of dissociated cells, microscopy, radiography, scintillation counting, etc. The presence and/or the level of a polymorphic polypeptide may also be detected and/or quantitated in any convenient assay format. In some embodiments, fluorescence-activated cell sorting (FACS) methods can determine the presence or absence of the different polymorphisms on cells isolated from the blood or other biological samples. See, e.g., for FcγRIIA: Böttcher, et al. (2005) J. Immunol. Meth. 306:128-36; and for FcγRIIA: Boruchov, et al. (2005) J. Clin. Immunol. 115:2914-23.

Additional references describing various protocols for detecting the presence of a target polymorphism include, but are not limited to, those described in: U.S. Pat. Nos. 6,703,228; 6,692,909; 6,670,464; 6,660,476; 6,653,079; 6,632,606; 6,573,049; the disclosures of which are herein incorporated by reference.

### Selecting Treatment Options

In view of the teachings of the present disclosure, the ability to predict responsiveness of subjects and therapeutic efficacy assurance treated with specialty drugs, e.g., to antibody maintenance therapy, allows a health care provider to assess and select various treatment options, e.g., selecting one therapy from a panel of several other therapeutic options available, that would likely have the most benefit for the patient, and conversely exclude use of treatments that would have insignificant benefit on treatment outcome. Accordingly, a method for selecting a treatment option for a disease treated by an ADCC-mediated drug can comprise:

determining a genotype of a human subject for one or more Fcγ polymorphisms affecting ADCC activity, wherein the human subject has an ADCC treatable disease; and

stratifying one or more treatment options based on the determined genotype of the Fcγ polymorphism, wherein the treatment options comprise at least antibody maintenance therapy for the ADCC treatable disease. Financial decisions based thereon are also provided.

In some embodiments, the stratifying of the various treatment options is done by comparing the determined genotype to a reference stratification that relates responsiveness to antibody and/or antibody maintenance therapy to genotypes of the FcγR polymorphism affecting ADCC activity. As noted herein, the stratification allows predicting the responsiveness to antibody maintenance therapy. Subjects whose responsiveness is excellent or good can be given the appropriate antibody maintenance therapy, while subjects whose responsive is weak or poor can be immediately switched, without further delay, to alternative therapies, such as chemotherapy or combination therapies that would benefit the patient.

As referenced in the present disclosure, the FcγR polymorphism affecting ADCC activity can be based on one or more FcγRIIA polymorphisms and/or one or more FcγRIIIA polymorphisms described above, particularly amino acid position 131 of FcγRIIA and amino acid position 158 of FcγRIIIA. Accordingly, in some embodiments, the treatments options can be selected based on the genotype groups. See U.S. Pat. No. 8,592,149, US patent publication 20100291549 and WO 201309047820, all of which are incorporated herein by reference; additionally, U.S. Provisional Patent Applications 62/332,315 and 62/322,325 dated May 5, 2016, both of which are incorporated herein by reference.

In some embodiments, the treatment option can comprise antibody maintenance therapy for a subject in genotype group (a), (b) or (c) in Table 2 of U.S. Pat. No. 8,592,149, given the likelihood of excellent to good responsiveness. Moreover, a subject in genotype group (a), (b) or (c) can be given induction therapy with an antibody therapeutic, followed by the antibody maintenance therapy. Decisions directed to financial coverage of treatment are also possible.

In some embodiments, the treatment option for a subject in genotype group (d) or (e) can exclude antibody body maintenance therapy as a treatment option. In some embodiments, a subject in genotype group (d) or (e) can be excluded from both induction therapy and maintenance therapy with an antibody. In some embodiments, the treatment options for a subject in genotype group (d) or (e) comprise a chemotherapy with a chemotherapeutic agent. In some embodiments, a treatment option for a subject in genotype group (d) or (e) includes chemotherapy for induction therapy as well as for maintenance therapy.

It is to be understood that the treatment option will depend on the disease or disorder being treated, as described herein, e.g., neoplastic disease, an autoimmune disease, an inflammatory disorder, a microbial infection, or allograft rejection, and that a person of skill in the art can select the appropriate treatment options available to the skilled artisan in view of the guidance and teachings of the present disclosure. See, e.g., Beers, et al. (eds. 2011) Merck Manual of Diagnosis and Therapy, Merck Publishing.

In some embodiments, the selection of a treatment option includes assessment of ADCC function or capacity. See, e.g., US patent publication 20100291549 and WO 201309047820, both of which are incorporated herein by reference; additionally, U.S. Provisional Patent Applications 62/332,315 and 62/322,325 dated May 5, 2016, both of which are incorporated herein by reference.

### Healthcare Management

Given the advantages of predicting responsiveness to specialty drugs, e.g., antibody therapy, and ability to select treatment options that have a likelihood of having a positive treatment outcome, the methods herein provide an additional benefit in assisting management of healthcare. For example, the methods herein allow a healthcare manager to make certain treatment options in order to achieve better therapeutic outcomes and reduce burden on financial resources.

Accordingly, in some embodiments, the present disclosure provides a healthcare management method for determining a healthcare payer coverage of antibody maintenance therapy for treating an ADCC treatable disease, the method comprising:

obtaining genotype information of a human subject having an ADCC treatable disease for a Fcγ receptor polymorphism affecting ADCC activity;

determining healthcare payer coverage of the antibody maintenance therapy based on the genotype information for the Fcγ receptor polymorphism.

In some embodiments, determining health payer coverage can comprise (a) comparing the genotype information to a reference stratification relating responsiveness to one or more antibody maintenance therapies to genotypes of the Fcγ polymorphism, (b) measuring ADCC function or capacity, or (c) using both information, as described throughout the present disclosure.

In some embodiments, the reference stratification can comprise data stored in a computer memory. In some embodiments, the comparing of the genotype information to the reference stratification can be carried in a computer.

In some embodiments, the method further comprises determining a treatment outcome for the antibody maintenance therapy. Thus, a treatment outcome that is weak or poor response can be a basis for not covering the maintenance therapy while a treatment outcome that is excellent or good can be a basis for approving coverage of the maintenance therapy.

Similar to the other methods described herein, the Fcγ receptor polymorphism affecting ADCC activity can be based on one or more FcγRIIA polymorphisms and/or one or more FcγRIIIA polymorphisms described above, particularly amino acid position 131 of FcγRIIA and amino acid position 158 of FcγRIIIA. Accordingly, in some embodiments, determining coverage can be selected based on the genotypes a subject presents and the corresponding genotype responsiveness established for various specialty drugs.

In some embodiments, the method further comprises determining a treatment option, as described in the present disclosure.

In some embodiments, the determining of coverage, the comparing of the genotype information, treatment outcome, and the treatment options can be reported in electronic, web-based, or paper form to the subject, a health care payer, third party payer, a health care provider, a physician or a government office.

### Reagents Devices and Kits

The present disclosure also relates to reagents, devices and kits thereof for practicing one or more of the above-described methods. For example, kits may comprise one or more elements for genotyping a patient to identify a polymorphism or genotypic variation, or gene deletions or duplications, and one or more elements for genotyping a patient to identify a FcγRIIIA polymorphism. Such elements may be, e.g., oligonucleotides, e.g., for PCR and/or sequencing the corresponding gene loci, for hybridization of the gene loci or etc., or as another example, antibodies, e.g., an antibody specific for the H¹³¹ or R¹³¹ allele of FcγRIIA, and an antibody specific for the V¹⁵⁸ or F¹⁵⁸ allele of FcγRIIIA. Additionally, or alternatively, kits may comprise one or more elements for detecting and measuring cells in a human sample, i.e. cells that are targeted for depletion and/or repopulation by an antibody induction therapy, for e.g., used to treat an ADCC-treatable disease. Such elements may include, e.g., antibodies, e.g., an antibody that is specific for a marker on the targeted cell, an antibody that is specific for a larger population of cells that comprise the targeted population, etc., a vital dye for determining cell viability, etc. The kit may further comprise a reference that correlates a genotype in the patient and/or the extent of target cell depletion and/or repopulation in a patient with patient groups having known responsiveness to the antibody maintenance therapy.

In addition to the above components, the subject kits will often further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a remote site. Any convenient means may be present in the kits.

### Workflow Involved: Exemplary Klaritos RA Platform

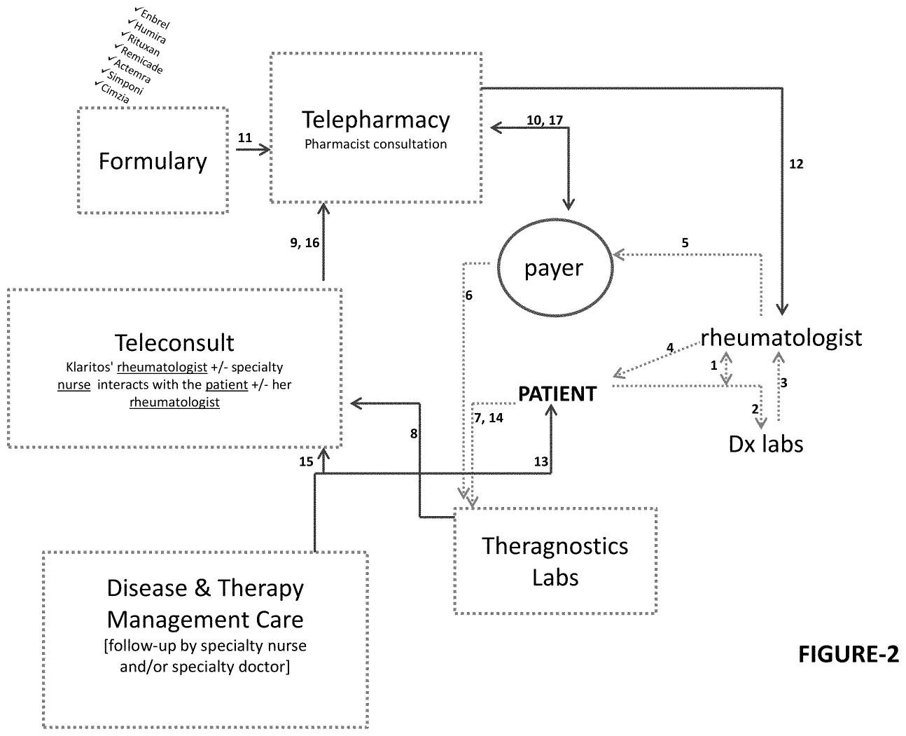

A patient with significantly severe joint pain and early morning stiffness is referred to a rheumatologist for further evaluation (FIG. 2; step-1). The specialist requests for diagnostic procedures including X-ray exams of hands, wrists, and knees, as well as blood tests that include rheumatoid factor (RF) and anti-CCP levels (step-2). Based on the diagnostic procedures (step-3), the rheumatologist diagnoses that the patient has moderate to severe RA, and informs the patient (step-4) and the payer for determining appropriate treatment decisions (step-5). These are the currently followed routine procedures by a rheumatologist and payers. Additional workflow involving an exemplary Klaritos platform is provided herein.

The payer uses Klaritos platform and its disease-specific PDP for the selection of specialty drugs and treatment procedures, and in fact, all RA patients covered by the payer in the United States are asked to go through Klaritos platform for the next steps in treatment. The platform comprises, e.g., all, or at least some, of (i) theragnostic labs, (ii) formulary; (iii) telepharmacy; (iv) teleconsult room; (v) disease and therapy management care (FIG. 2).

Accordingly, the payer refers the patient to the theragnostic labs of Klaritos platform (step 6), and the patient provides a blood sample for further analyses (step 7). The patient is then evaluated to determine which tests are performed to evaluate the status and progression of disease individually. Certain tests may be performed to eliminate various possibilities, others to confirm others, and still others to determine disease progression and baseline status.

Based on the theragnostic analyses specifically indicated for testing for the patient, she is tested for and determined seropositive with the following characteristics: IgG-RF⁻; IgG-ACPA⁺; fibrinogen immune complex⁻; very high levels of TNF-α as determined by the EPP/AAI assays. A blood sample taken from the patient is used to prepare genomic DNA, which is amplified by PCR using pairs of primers specific for the FcγRIIA, FcγRIIIA, and FcγRIIIB loci (see, e.g., Lehrnbecher, et al. (1999) Blood 94:4220-32). The results indicate that the patient is homozygous H at residue 131 of FcγRIIA, homozygous V at residue 158 of FcγRIIIA, and homozygous NA1 of FcγRIIIB. These results are examined by the Director of Theragnostic Labs, and an in-house rheumatologist. Baseline characteristics of B-cell subsets (naïve, memory, and plasma B-cell subsets) are also determined, e.g., by minimal residual disease flow cytometry (MRD-FC; see below). By way of teleconsultation, all of these results are further discussed with the patient and her rheumatologist (step-8). Further, the in-house rheumatologist provides an opinion to the patient's rheumatologist regarding: (a) whether to proceed with antibody maintenance therapy using rituximab, and if so, under what regimen, and (b) whether to withhold methotrexate administration or administer on as-needed basis. Based on the IgG-RF and ACPA levels, reference indices relating genotype groups to (a) disease severity, and (b) category of treatment response to antibody maintenance therapy, and it is predicted that the patient will have an extremely severe disease course, however, an excellent candidate for rituximab maintenance therapy. The patient's rheumatologist and the in-house rheumatologist agree on the selection of therapy and treatment guidance protocols: rituximab induction therapy. The patient undergoes induction therapy with rituximab, a therapy comprising rituximab and methotrexate. The patient is prescribed a 4-week course of rituximab induction therapy (375 mg/m² once per week).

Electronic prescription is sent to telepharmacy (step-9), which initiates prior authorization request with payer, and the payer duly authorizes through electronic means (step-10).

The specialty pharmacist requests the drugs from the drug formulary (step-11), and the drugs are shipped to patient's physician's office (step-12; “white bagging” process). The patient is prescribed a 4-week course of rituximab induction therapy (375 mg/m² once per week). The drugs are administered by the patient's rheumatologist with the location and time recorded, providing location-specific and time-specific authentication of dosing.

Rheumatology specialist from Disease and Therapy Management care is in touch with the patient and provides guidance and follow-up (step-13). Two months after induction therapy, the patient provides blood sample to Theragnostic Labs (step-14) for the analyses of: IgG-RF, ACPA, IgA-RF, ACPA levels, B-cell subsets by MRD flow cytometry. The RF and ACPA levels have significantly reduced 60% less compared to the pre-treatment levels. The flow cytometry results indicate <5 CD19⁺ naïve and memory B-cells per microliter, <10 CD19⁺ CD27⁺⁺, CD38⁺⁺ plasma B-cells, i.e., substantial depletion with the anti-CD20 antibody therapy, correlating to excellent therapeutic response (Step-14). All these results are discussed with the patient and her rheumatologist (step-15) through teleconsult process. The rheumatologists agree that the patient responds excellently to rituximab induction therapy based on the B-cell depletion profiles, disease severity indices. The patient's rheumatologist then prescribes rituximab maintenance therapy: a 2-week course of therapy (375 mg/m² once per week), to be administered at 3-month intervals for the first 4 cycles after the induction therapy (375 mg/m² once per week), and then an as-needed rituximab regimen as determined by B-cell depletion profile. Methotrexate, known to have serious side effects in the patient, is determined not to be necessary and hence not administered.

Electronic prescription for rituximab maintenance therapy is sent to telepharmacy (step-16), which initiates prior authorization request with payer, and the payer duly authorizes through electronic means (step-17).

The specialty pharmacist requests the drugs from the drug formulary (step-11), and the drugs are shipped to patient's physician's office (step-12; “white bagging” process). The drugs are administered by the patient's rheumatologist.

The patient is in full clinical remission in Year-1 and Year-2.

### Telemedicine; Telepharmacy; Telehealth

Telemedicine, which includes at least near-real-time teleconference between medical professionals, will typically include a plurality of locationally-disperse specialists, who may have disparate or overlapping expertises, to discuss a patient's case. Communications will typically be in real-time, and each participant has access to some or all of relevant features of diagnostic evaluations, medical record and history, past treatment, perhaps insurance coverage details and options, and other relevant details, both medical and treatment-related. Included may be specialist doctors, e.g., rheumatologist, neurologist, immunologist, and likely the patient's primary treating physician. The telemedicine group may be, or include, substantial overlap with, the Disease and Treatment Management team, which may include specialty nurses and other healthcare professionals. The communication systems will require substantial security to handle confidential patient information and data, as well as have means to ensure only appropriate persons have access to the system and data.

Telepharmacy will typically include connection, often real-time, which allows communication between the prescribing physician(s), which may include disease specialist on the Disease and Treatment Management team, and the pharmacist, who fulfills the prescription. Because of the high cost of the specialty drug, typically the payer is also connected whose approval or pre-approval is generally needed before the drug is dispensed or delivered to the patient. The drug may be delivered directly to the patient or to someone who is responsible for ensuring proper administration of the drug, one who typically ensures and documents both timing and dosing for patient therapeutic adherence. In some cases, the drug is provided to the guardian of the patient, e.g., where the patient is pediatric or geriatric, or may need assistance in healthcare needs.

The payer may be the insurer, or there may be one of various intermediaries including, e.g., a pharmacy benefits management (PBM) entity, a specialty pharmacy, or others who may be included to coordinate the prescribing, ordering, stocking, delivery, drug administration, and patient therapeutic adherence verification with use of a drug, e.g., specialty drug. In other circumstances, e.g., in a socialized medicine or single payer health system, e.g., healthcare systems in Canada, EU countries, Scandinavian countries, the intermediaries may be fewer and may include or overlap with other social services entities which may include aspects of healthcare provision or monitoring by trained healthcare professionals or which may include forms of nursing care and the like.

The invention conceives of further remote communication networks directed to ensure that the patient/guardian is provided access to treatment decision-making process. The goal is to minimize or eliminate hurdles that prevent patient therapeutic adherence.

The invention further conceives of telehealth electronic payment system for efficient and timely management of money transfers between payers, employers, patients, and pharmaceutical companies. The system may be made available via a Mobile App and enables patients to pay their copays (co-insurance). Such a payment model, inherently guided by theragnostic methods, may facilitate automatic monitoring and determination of patient therapeutic adherence and implementation of therapeutic efficacy assurance without involving any other third party. This allows timely determination of patients' remission and excellence as well as transfers of refunds where appropriate. The assurance company may facilitate collection of copay (coinsurance) from patients, and payment for acquisition of drugs. The company may manage therapeutic efficacy assurance as its own fund.

### Theragnostic Guidance Versus Diagnostic Evaluation

Theragnostics entail various evaluations of the patient to determine disease status; help match an appropriate drug to the patient; and provide therapeutic guidance in how best to treat that individual patient with the matched drug. In contrast to “personalized medicine” which typically uses a static evaluation of patient status, theragnostics consider the dynamic nature of temporal and longitudinal follow-up of disease progression, mechanism of action of drug, pharmacological features, e.g., absorption, distribution, metabolism, excretion (ADME) for the drug. Thus, e.g., where the mechanism of action may be immunologically mediated, there will be surrogate readouts. Theragnostic evaluation may consider the immunological function of the individual upon dosing, with feedback used to evaluate whether the treatment is effective, or if treatment is approaching effectiveness. The individualized nature of the evaluation will allow dosing to be matched temporally with the individual's tolerance to the drug. Moreover, because the mechanism may be dynamic, changes can be tracked to determine whether the desired endpoint may be reached before toxicity or other limitations are reached to prevent the desired endpoints. With such dynamic tracking, the ability to project outcomes will improve.

Theragnostic criteria are used to evaluate the current status of the individual patient, to predict shorter or longer-term progression of disease; to determine what is an appropriate drug for that individual patient, to determine who is identified as a therapeutically ineligible patient for a particular drug, and to determine whether a particular drug is achieving its appropriate mechanism to treat disease, and how quickly it may lead to better treatment results, e.g., complete remission, or alternatively if failing, how quickly it is failing. In this latter case, a quicker switch to an alternative may be effected, perhaps within a limited window-of-effective-opportunity, which may have significantly desirable effects on treatment and economic outcomes. In other circumstances, it may be possible to combine drugs having two different mechanisms of action together to achieve desired therapeutic effect. In other situations, where the mechanism of action is not working, perhaps supplementing or treating that deficiency may reconstitute the normal mechanism sufficiently to achieve desirable treatment outcomes.

In particular, theragnostics allow individualized treatment to achieve significantly better treatment response, e.g., clinical remission or excellent response, and doesn't rely upon a presumption that all patients are uniform in response. Thus, the individualized diagnosis and therapy are different from the old style “personalized medicine” which accounts overly inclusive patient pool for treatment.

### Therapy Guideline Adherence

Due to significant advances in our understanding in the pathophysiology of several autoimmune disease, cancers, inflammatory disorders, etc., well established disease-specific treatment guidelines are adopted to monitor disease activity and change treatment in a timely manner if a preset target is not reached. This is herein referred to as Therapy Guideline Adherence (TGA).

In RA, e.g., many guidelines and recommendations on optimal care have been developed to help clinicians choose the best diagnostics and therapeutic strategies. Current guidelines proposed by American College of Rheumatology (ACR) and the European League Against Rheumatism (EULAR) are based on tight control principles, where monitoring of disease activity and modifying treatment are essential if a preset target is not reached. Such tight therapeutic guideline adherences can result in lower disease activity or possibly even disease remission, and less functional damage and deformity compared with typical care guidelines. However, current standard practices adopt suboptimal TGA. TGA percentages varied considerably among parameters, suggesting suboptimal TGA on at least some guideline recommendations. TGA also varied among rheumatologists, and several rheumatologist and patient-related determinants (e.g., patient sex, number of DMARD options already exhausted, presence of erosions, RF or anti-CCP positivity,) were found to be related to rheumatologists' guideline adherence. TGA varied from 21-72% in one study, and 24-90% in other studies. See Harrold, et al. (2016) Arthritis Res. and Ther. 18:94; Lesuis, et al. (2016) Rheumatism and Musculoskeletal Diseases Open 2:e000195; and Harrold, et al. (2012) Arthritis and Rheumatology 64:630-638. Adherence among rheumatologists depended on number of patient visits, type of DMARDs used, disease activity and prognosis, and rheumatology practice capability to assess disease activity and engage in shared decision making with patients. In addition, patient's economic or insurance status, employment status, cost of the specialty drugs, guidelines set forth by payers are expected to greatly influence TGA as it will have direct bearing whether a rheumatologist would want to prescribe a specialty drug or switch to another specialty drug. Streamlining these inefficiencies can greatly enhance TGA, or at least ensure that any divergence from TGA is reasoned, intentional, and not inadvertent.

In an integrated delivery, treatment, and payment model, e.g., Klaritos model, TGA can be improved through adoption of (a) theragnostic evaluation, (b) efficacy and financial assurances, and (c) patient therapeutic adherence. In one embodiment, novel theragnostic evaluations for the effective selection and treatment strategies with specialty drugs can significantly improve TGA. In one instance, TGA is theragnostics-based, e.g., theragnostic evaluation procedures are used a priori to identify and administer the right specialty drug in a given patient, e.g., instead of fail-this-one-first treatment strategy in RA, such that remission or excellent response can be achieved in very early RA and early RA patients, which leads to better TGA, preferably 60-80%. In some instances, 80-90% TGA is achieved; and in yet other instances, 90-100% TGA is achieved.

Among other reasons stated above, for e.g., patient's economic and employment status indirectly reflects on TGA rates. Thus, in an embodiment, TGA is favorably influenced by assurances, e.g., providing efficacy and financial assurances to patients and payers will enable physicians to adopt higher TGA rates, e.g., 70% or preferably 80-90% or higher. In yet another embodiment, better patient therapeutic adherence and compliance, and hence much better therapeutic outcomes, can positively impact better TGA rates. In yet another embodiment, improved TGA leads to improved patient response to treatment, which will lead to advantages in assurance, e.g., efficacy assurance or financial assurance. These will have positive financial effects for the individual patients and for the patient pools, e.g., payers or health insurers.

The Klaritos platform will also provide medical records which track both the diagnostic and theragnostic evaluations of individual patients, which are combined into a database with a plurality of records. The records and databases can be sorted into disease-specific subsets, or the disease-specific databases can be combined across different diseases for a larger database, which can be deconvoluted back into smaller disease-specific database subsets. The databases will typically include: patient identification information, patient history information, theragnostic evaluation information, therapeutic and therapy guidance information, treatment information such as patient therapeutic adherence, response evaluation (both interim and longer term; with focus on remission and excellent response rates, or low or extremely low response rate subsets), therapeutic assurance information, financial assurance information, health insurance and drug delivery or prior authorization information, and related information which allows tracking of medical aspects of the patient case, which may be linked to financial and other insurance or assurance related aspects of patient activities. The databases may be separated into regional, geographical, national (e.g., US, Canada, European, UK, Scandinavian, etc.), or by other parameters as desired. The database may start small, e.g., 100 patients, and grow to 500, 10000, 200000, and so on, and as the size grows the statistical power of contained data also grows. Some databases will be composed primarily of disease-specific databases, others may combine one or more disease-specific databases, and others may be combined with other databases comprising other databases. Typically the combined databases may be deconvoluted to separate back out various components, e.g., the theragnostic-guided treatment cases, and allowing tracking of response results of various treatment strategies. Because the Klaritos databases will typically track realtime activities, the databases will allow dynamic tracking of patient therapeutic adherence, e.g., timing and accuracy of drug dosing, tracking of when theragnostic evaluations are performed to track Therapy Guidelines Adherence (TGA), and to dynamically compare new treatment strategies with prior standard of care (SOC) responses. Thus, these databases are continuously iterative (evolving, and improving as additional patients are added), and inherently archivable. Ultimately, the database in a disease-specific group may include a large fraction, e.g., 20%, 40%, 60%, or more, of the entire disease pool within the system, which may include a regional or other mostly inclusive category. The database also provides insights into how a new drug will perform where it uses a related mechanism of action to another drug, e.g., anti-TNF-alpha therapies for RA, within the Klaritos formulary, and will allow selection of patients for fast internal clinical or comparison trials, e.g., with new or similar drugs or with modifications to therapeutic use of existing drugs.

The Klaritos integrated treatment model, when implemented, can address the dual problem of patient therapeutic adherence (PTA) and TGA by rheumatologists simultaneously. The Klaritos disease-specific databases track PTA to a therapy as a condition for efficacy (financial) assurance. By this process a patient-specific tailored treatment recommendation is available to rheumatologists that influences favorably TGA. Thus, more patients are treated with the most effective specialty drugs, e.g., biologic DMARDs, which leads to expanded patient therapeutic adherence resulting in improved outcomes and demonstrably lower healthcare costs.

Any discrepancy between on-going treatment strategy of a patient and accepted guidelines can be quickly identified and addressed by Klaritos platform. Conversely, any new or modified treatment strategies, possibly developed by Klaritos theragnostic process, can quickly be adopted into state-of-the-art treatment guidelines based upon statistically acceptable available data within the databases.

Through the theragnostic component of the Klaritos platform physicians will have tangible evidence of the most effective biologic DMARD for subsets of patients prior to the initiation of and during the course of therapy. Ongoing monitoring and guidance of the patient based on theragnostics and consults with the Klaritos rheumatologists will keep the physician and patient informed of the most appropriate therapy during treatment, and provide insight on adjustments to achieve optimal outcomes. Individual patient treatment histories are contained therein, and populational comparisons of treatment strategies can be readily performed and tracked. The data supporting high response rates, e.g., remission or excellent response, can serve to further induce new patients to adhere to the effective treatment course.

### Efficacy Assurance

Efficacy assurance consists of therapeutic efficacy assurance (TEA) and financial assurance (FA). TEA refers to an assurance of achieving significantly better therapeutic efficacy in a given patient or subset(s) of patients, within a reasonable time-frame, e.g., 1 month, 2-3 months; this assurance is provided to patients, or payers, and employers. Specifically, TEA is not therapeutic risk assurance; that is, this does not cover risks and side effects associated with the drugs. Financial assurance refers to a form of money-back guarantee, e.g., co-insurance amount, if the therapy has not achieved desired therapeutic outcome within a reasonable time-frame, e.g., 2-3 months; this assurance is provided to patients, and in some instances, it may also be provided to payers and employers. Such assurance is theragnostics-guided in specific disease indications, provided the patient establishes and maintains excellent PTA therapeutic adherence rate. Both TEA and FA are inter-related: it is essentially a warranty that some or all of the cost of drug, with or without treatment costs, will be returned if the patient does not achieve a designated treatment response. In an autoimmune disease such as rheumatoid arthritis, the designated target response is likely to be at least, e.g., excellent response or more preferably remission. In a typical example, the treatment may be to treatment with a monoclonal antibody therapy such as rituximab induction therapy, as described above. Should the patient adhere to the treatment parameters, and the selected treatment does not achieve its intended therapeutic effect, some or all of the out-of-pocket cost of drug will be returned to the patient, e.g., all of the patient drug co-insurance costs. However, if the patient achieves excellent response or clinical remission to treatment, then the therapeutic efficacy assurance is satisfied and thus she will not get any co-insurance amount back. This can serve as an incentive to patient therapeutic adherence as a condition of the financial assurance, e.g., money-back guarantee.

This does require that the individual patient actually adhere to the treatment protocol, both regarding timing (e.g., within a 6-hour treatment window to take the drug) and dosing. The treatment may be a predesignated treatment protocol, where all aspects of the therapy are specified before the treatment is begun and no adjustments are introduced thereafter; alternatively, patient therapeutic adherence criteria might be adjusted during the course of treatment, e.g., adjusted by disease specialist, e.g., according to theragnostic criteria and theragnostic evaluation of the individual patient while the treatment schedule is underway.

### Efficacy and Financial Assurance to Payers

Klaritos model provides disease-specific therapeutic efficacy and financial assurances to payers and employers. Such assurances are provided not on a single patient basis but more on a population basis, e.g., for the entire RA population administered for a payer or an employer within a defined time period, e.g., 12, 18, 24 months. Depending on the percentage of remission or excellent response achieved in that population, payers can notice significant pharmaco-economic benefits, e.g., significantly reduced total direct costs, which is a summation of specialty drug costs and total direct medical costs (hospitalization, surgery, etc.).

With disease remission, drug costs for certain periods e.g., 3-6 months or 12 months are eliminated, as the drug is no longer needed when remission is achieved, and hence direct medical costs reduce proportionally and concomitantly. Likewise, with excellent response, the drug costs are significantly reduced, e.g., reduced dosing, dosing schedule, less therapy changes and thus, direct medical costs will also reduce because of significant reduction of the disease progression or disease severity.

The following is an exemplary analysis for Rheumatoid Arthritis (RA). Table AA is a 2-dimensional matrix that shows the financial assurance in terms of percentage cost savings potentially achieved by employers and payers when patients achieve remission and excellent response through Klaritos model. By identifying and selecting the most appropriate drug therapy for a patient or subsets of patients, a certain percentage of patients in a given population will achieve complete remission and others will achieve an excellent response within a defined time period. These objective treatment endpoints are defined and accepted by the medical field. See, e.g., the American College of Rheumatology (ACR) website www.rheumatology.org; EULAR guidelines; Health Assessment Questionaire (HAQ); or DAS scoring scale.

Each cell in the matrix provides an estimate of the percentage of overall combined (specialty) drug cost and direct medical cost savings, and thus, that is the financial assurance provided to employers and payers for its RA disease-specific population (e.g., 20,000 or 250,000 patients).

Percentage cost savings are provided in this matrix for remission and excellent response rates achieving 10, 20, 30, and 40%, and can be compared in various combinations for a RA disease population managed by a payer. For example, in a patient population where 30% of patients achieve remission and 30% achieve an excellent response, the total direct cost savings is 39.6%. In a patient population where 20% of patients achieve remission and 40% achieve an excellent response the total direct cost savings is 35.2%. See Table AA.

The percentage cost savings for various rates of remission and excellent response were developed based on inferences drawn from multiple sources, and published data on the cost of RA specialty drugs and the direct medical costs incurred by patients receiving specialty drugs based on inflation-adjusted healthcare cost figures.

Underlying assumptions include: (a) RA patients in remission will avoid 100% of drug costs and 75% of direct medical costs; (b) those achieving excellent response will avoid 30% of drug costs and 60% of direct medical costs. In patients achieving excellent response drug costs are reduced because of (a) fewer therapy switches and associated wasted drug cost, (b) fewer patients requiring dose escalation, (c) the potential to taper medication more rapidly in some patients, and (d) less drug wastage in drug naïve patients new to therapy because of theragnostic guidance.

The data used for calculating the cost of RA specialty drugs and associated direct medical costs were based on a publication by Gleason, et al. (2013) J. Managed Care Pharmacy 19:542-548 and inflation-adjusted to 2016 dollars using PricewaterhouseCoopers (PwC) Institute of Medical Cost Trends. These calculations indicate that on average RA specialty drug costs are $27,884 per patient per year, and the average annual direct medical cost for each patient is $24,728 with a total direct cost at $52,612 per patient.

The per patient cost basis can then be used to project the total specialty drug costs and direct medical costs for any population size, e.g., RA patient population of an employer or payer, being treated with specialty drugs. By applying variable remission and excellent response rates as noted above the total cost savings can be calculated.

For example, in populations of 7,500 and 25,000 RA patients treated with specialty drugs the following scenarios are average baseline costs for the major elements of care prior to initiating Klaritos system:

| | RA patients treated with specialty drugs | |
|---|---|---|
| | 7,500 | 25,000 |
| Specialty drug cost ($M) | 209.1 | 697.1 |
| Direct medical costs ($M) | 185.5 | 618.2 |
| Total direct cost ($M) | 394.6 | 1315.3 |

After implementing the Klaritos system in a scenario where (a) 30% of the population achieve remission, and (b) another 30% achieve excellent response, then the estimated total direct cost savings is 39.6%. (Table AA) This translates to $156.3 M and $520.9 M in financial assurance in the above 7,500 and 25,000 patient populations, respectively.

A detailed example of specialty drug and direct medical cost dollar savings utilizing the Klaritos platform for a population of 7500 RA at various rates of remission and excellent response follows in Table AB and Table AC.

For the 7500 RA patients achieving 30% remission and 30% excellent response as described in Tables AB and AC cost savings gained are $104.3 M and $52 M respectively for a total savings of $156.3 M. Those patients in remission had $62.8 M reduction in specialty drug costs and $41.5 M reduction in direct medical costs. In the same population of patients those with excellent response had a $18.7 M reduction in specialty drug costs and $33.3 M reduction in direct medical costs.

TABLE AB
Cost Savings: Patients in Remission (%)

Remission Response Rates (%)

| Cost Savings ($M) | 10 | 20 | 30 | 40 |
|---|---|---|---|---|
| Specialty Drugs | 20.9 | 41.8 | 62.8 | 83.8 |
| Direct medical Costs | 13.9 | 27.8 | 41.5 | 55.8 |
| Total Cost savings | 34.8 | 69.6 | 104.3 | 139.6 |

TABLE AC
Cost Savings: Patients with Excellent Response (%)

Excellent Response Rates (%)

| Cost Savings ($M) | 10 | 20 | 30 | 40 |
|---|---|---|---|---|
| Specialty Drugs | 6.3 | 12.5 | 18.7 | 24.9 |
| Direct medical Costs | 11.1 | 22.2 | 33.3 | 44.6 |
| Total Cost savings | 17.4 | 34.7 | 52 | 69.5 |

Note:
Estimated time period: 18 months after initiating Klaritos Program, given that the treatment period may last 2-6 months, and the pharmacoeconomic benefits are realized in the following months.

### Product Differentiation and Market Enrichment

Several approved drugs, e.g., 15-20 drugs or more, and mostly specialty drugs, may be commercially available for treatment of a particular disease indication, e.g., rheumatoid arthritis, relapsing-remitting multiple sclerosis. In the current treatment model, not all drugs are therapeutically effective in a patient or subset(s) of patients, and this is information may not be known a priori. Currently, features such as therapeutic efficacy assurance including the associated financial assurance, theragnostic guidance in a patient or subset(s) of patients, disease and therapy management care, and product differentiation of specialty drugs are not provided by prescription drug plans (PDPs), PBMs or specialty pharmacies. In such a crowded market scenario, and a step-therapy (e.g., fail-this-one-first) treatment scenario that is adopted by payers and providers, differentiating a particular drug from others have significant advantages to all stakeholders: patients, payers and employers, healthcare providers, and pharmaceutical companies. For instance, the annual net sales of the drug can be considerably higher, e.g., 2-fold or 5-fold higher, if the product is subject to product differentiation. Employers and payers may be willing to approve and administer the drug even if it is priced higher, e.g., 25% more, or 100% more than the alternative drugs.

When a specialty drug enters the market through a theragnostics-guided healthcare supply chain and delivery model, because of the therapeutic guidance that the drug will have, the drug is most probably expected to differentiate itself from other IP-protected drugs as well as its biosimilars or generics in the market in regards to efficacy, safety and toxicity profiles. Such product differentiation is theragnostics-guided, and this feature may be exploited by (a) a prescription drug plan, (b) a drug formulary, (c) a specialty pharmacy, (c) a payer, (d) an employer, (e) a pharmaceutical company, (f) a diagnostic company, (g) a drug distributor, or (h) a healthcare provider. Such product differentiation may be to do with efficacy, therapeutic value, economic value, financial value, pricing, patient subsets or segment(s), etc. Market enrichment refers to theragnostics-guided identification of a treatable patient, treatable subset(s) of patients, a treatable segment of patient market in a particular disease indication for the purposes of distribution, delivery of a drug, and treatment with a drug, with an objective of achieving better therapeutic and economic outcomes. This method selectively avoids patients who are considered not eligible for a particular therapy. Treatable herein means treatment-eligible patients. This market enrichment feature may be exploited by (a) a prescription drug plan, (b) a drug formulary, (c) a specialty pharmacy, (c) a payer, (d) an employer, (e) a pharmaceutical company, (f) a diagnostic company, (g) a drug distributor, or (h) a healthcare provider.

One way to accomplish this is to achieve enrichment of a hidden but treatable patients, segments of patients, or subsets of patients (e.g., collectively called addressable market) who otherwise are not eligible for treatment or prior authorization. Such an enriched market becomes a significant and addressable market size for the drug, e.g., specialty drug. Another way to accomplish is to identify subset(s) of patients who will respond better to a therapy, e.g., a specialty drug, based on the mechanism of action of the drug. This may initially be construed as an aspect leading to market fragmentation, e.g., market minimization, for e.g., 25% of the total disease population. While the market size may come down, more patients from this defined albeit smaller market may be administered and thus leading to enhanced market share, e.g., 2-4 fold or perhaps more. This may effectively achieve nearly the same amount of net sales, if not more, as it would have otherwise in an all-corners market.

The following examples are offered by way of illustration and not by way of limitation. In addition to the example below, as taught through various sections of this invention, Klaritos and KlariPay platforms can be used to treat any disease or disease indication with a specialty drug.

### EXAMPLES

### Example 1: Workflow Involved in Klaritos Platform

A patient with significantly severe joint pain and early morning stiffness is referred to a rheumatologist for further evaluation (FIG. 2; step-1). The specialist requests for diagnostic procedures including X-ray exams of hands, wrists, and knees, as well as a blood test that includes rheumatoid factor (RF) and anti-CCP levels (step-2). Based on the diagnostic procedures (step-3), the rheumatologist diagnoses that the patient has moderate to severe RA, and informs the patient (step-4) and the payer for determining appropriate treatment decisions (step-5). These are the currently followed routine procedures by rheumatologist and payers. Additional workflow involving Klaritos platform is provided herein.

The payer uses Klaritos platform for the selection of specialty drugs and treatment procedures, and in fact, all RA patients covered by the payer in the United States are asked to go through Klaritos platform for the next steps in treatment. The platform consists of (i) theragnostic labs (theragnostic facility), (ii) formulary; (iii) telepharmacy; (iv) teleconsult room; (v) disease and therapy management care (FIG. 2).

Accordingly, the payer refers the patient to the theragnostic labs of Klaritos platform (step 6), and the patient provides blood sample for further analyses (step 7).

Based on the theragnostic analyses, the patient is seropositive with the following characteristics: IgG-RF⁻; IgG-ACPA⁺; fibrinogen immune complex⁻; very high levels of TNF-α as determined by the EPP/AAI assays. A blood sample taken from the patient is used to prepare genomic DNA, which is amplified by PCR using pairs of primers specific for the FcγRIIA, FcγRIIIA, and FcγRIIIB loci (see, e.g., Lehrnbecher, et al. (1999) Blood 94:4220-32). The results indicate that the patient is homozygous H at residue 131 of FcγRIIA, homozygous V at residue 158 of FcγRIIIA, and homozygous NA1 of FcγRIIIB. These results are examined by the Director of Theragnostic Labs, and the in-house rheumatologist. Baseline characteristics of B-cell subsets (naïve, memory, and plasma B-cell subsets) are also determined by minimal residual disease flow cytometry (MRD-FC; See Dass, et al. (2008) Arth. Rheum. 58:2993-2999; Vital, et al (2011) Arth. Rheum. 63:603-608). By way of teleconsultation, all of these results are further discussed with the patient and her rheumatologist (step-8). Further, the in-house rheumatologist provides an opinion to the patient's rheumatologist regarding: (a) whether to proceed with antibody maintenance therapy using rituximab, and if so, under what regimen, and (b) whether to withhold methotrexate administration or administer on as-needed basis. Based on the IgG-RF and ACPA levels, reference indices relating genotype group to (a) disease severity, and (b) category of treatment response to antibody maintenance therapy, and predicts that the patient will have an extremely severe disease course, however, an excellent candidate for rituximab maintenance therapy. The patient's rheumatologist and the in-house rheumatologist agree on the selection of therapy and treatment guidance protocols: rituximab induction therapy. The patient undergoes induction therapy with rituximab, a therapy comprising rituximab and methotrexate. The patient is prescribed a 4-week course of rituximab induction therapy (375 mg/m² once per week).

Electronic prescription is sent to telepharmacy (step-9), which initiates prior authorization request with payer, and the payer duly authorizes through electronic means (step-10).

The specialty pharmacist requests the drugs from the drug formulary (step-11), and the drugs are shipped to patient's physician's office (step-12; “white bagging” process). The patient is prescribed a 4-week course of rituximab induction therapy (375 mg/m² once per week). The drugs are administered by the patient's rheumatologist.

Rheumatology specialty from Disease and Therapy Management care is in touch with the patient and provides guidance and follow-up (step-13). Two months after induction therapy, the patient provides blood sample to Theragnostic Labs (step-14) for the analyses of: IgG-RF, ACPA, IgA-RF, ACPA levels, B-cell subsets by MRD flow cytometry. The RF and ACPA levels have significantly reduced 60% less compared to the pre-treatment levels. The flow cytometry results indicate <5 CD19⁺ naïve and memory B-cells per microliter, <10 CD19+CD27++, CD38++plasma B-cells, i.e., substantial depletion with the anti-CD20 antibody therapy, correlating to excellent therapeutic response (Step-14). All these results are discussed with the patient and her rheumatologist (step-15) through teleconsult process. The rheumatologists agree that the rituximab induction therapy puts the patient's disease in excellent treatment response category based on B-cell depletion profile, disease severity indices. The patient's rheumatologist then prescribes a 2-week course of therapy (375 mg/m² once per week), to be administered at 3-month intervals for the first 4 cycles after the induction therapy (375 mg/m² once per week), and then an as-needed rituximab regimen as determined by B-cell depletion profile. Methotrexate is not administered as part of the maintenance treatment strategy.

Electronic prescription for rituximab maintenance therapy is sent to telepharmacy (step-16), which initiates prior authorization request with payer, and the payer duly authorizes through electronic means (step-17).

The specialty pharmacist requests the drugs from the drug formulary (step-11), and the drugs are shipped to patient's physician's office (step-12; “white bagging” process). The drugs are administered by the patient's rheumatologist.

The patient is in full clinical remission in Year-1 and Year-2.

What is claimed is:

1. A method providing assurance for a specialty drug treatment, comprising:
a) selecting a specialty drug by a theragnostic evaluation of a patient for treatment of a chronic disease or disorder; and
b) providing efficacy assurance or financial assurance.

2. The method of claim 1, further comprising
a) selecting the specialty drug from a panel of available drugs in a drug formulary;
b) treating the patient with an appropriate specialty drug; or
c) providing efficacy and financial assurances.

3. The method of claim 1 applied to a plurality of individual patients.

4. The method of claim 1, wherein the theragnostic evaluation provides:
a) therapeutic appropriateness in the patient or group of patients;
b) therapeutic guidance in the patient or group of patients;
c) therapeutic effectiveness in the patient or group of patients; and
d) selection of alternative therapeutic strategy in the patient or group of patients in the event of contraindication or failure of therapy.

5. The method of claim 1, wherein the theragnostic evaluation further:
a) stratifies a disease population into one or more distinct subsets based on immunological subtype(s);
b) stratifies a disease population into one or more distinct subsets based on disease severity;
c) achieves significant therapeutic response in one or more subtypes of disease by administering a specialty drug;
d) achieves significant therapeutic response in a subject categorized according to a set of immunological subtypes by administering a specialty drug;
e) achieves significant therapeutic response in multiple subset(s) of the disease population; or
f) includes evaluation of responsiveness to drug during treatment.

6. The method of claim 1, wherein the efficacy assurance is provided:
a) for a specialty drug for the treatment of a disease, disorder, or cancer; or
b) by using a theragnostic evaluation comprising use of a biomarker, diagnostic procedure, companion diagnostic procedure, or combination thereof.

7. The method of claim 1, wherein the efficacy assurance is provided to:
a) an eligible patient selected from a disease population;
b) an eligible subset of patients selected from a disease population; or
c) all eligible patients selected from a disease population.

8. The method of claim 2, wherein the therapeutic efficacy assurance is provided to an eligible patient, payer, or employer.

9. The method of claim 8, wherein the payer is a private payer or government payer.

10. The method of claim 2, wherein the financial assurance is provided to the eligible patient, payer, or employer.

11. The method of claim 2, wherein the financial assurance involves:
a) full or partial money-back guarantee of co-insurance to the eligible patient; or
b) full or partial money-back guarantee to the payer or the employer who pays for the specialty drug.

12. The method of claim 1, wherein the specialty drug is:
a) approved by a disease specialist;
b) intended for treating an autoimmune disease, inflammatory disorder, a rare disease, a cancer indication, or microbial infection;
c) further delivered or dispensed for administration to the patient;
d) a biotech product;
e) an oral or injectable formulation; or
f) subject to risk evaluation and mitigation strategies from drug manufacturer(s).

13. An entity selected from:
a) a pharmacy;
b) a pharmacy benefits management entity;
c) an employer or insurance entity, including a health insurer, commercial health insurer, or public/government health insurer;
d) a healthcare provider entity, including a clinic, hospital, outpatient facility, specialty clinic, or physician's office;
e) a pharmaceutical company; or
f) a diagnostic company;
which uses or pays for the method of claim 1.

14. A method providing assurance-based prior authorization of a specialty drug, comprising:
a) using a theragnostic evaluation of a patient for treatment of a chronic disease or disorder; and
b) making an assurance-based prior authorization decision for payment for the specialty drug.

15. The method of claim 14, further comprising;
a) selecting the specialty drug from a panel of available drugs in a drug formulary;
b) dispensing the specialty drug by a specialty pharmacy to the patient, or
c) delivering the specialty drug to the patient.

16. The method of claim 14, wherein the prior authorization is provided:
a) for a specialty drug for the treatment of a disease, disorder, or cancer; or
b) by using a theragnostic evaluation comprising use of a biomarker, diagnostic procedure, companion diagnostic procedure, or combination thereof.

17. The method of claim 14, wherein the prior authorization involves:
a) approval of the specialty drug that was originally prescribed by a disease specialist of the patient; or
b) substitution of the specialty drug that was originally prescribed by a disease specialist of the patient with an alternate specialty drug or non-specialty drug by a Disease and Therapy Management specialist.

18. An entity selected from:
a) a pharmacy;
b) a pharmacy benefits management entity;
c) an employer or insurance entity, including a health insurer, commercial health insurer, or public/government health insurer;
d) a healthcare provider entity, including a clinic, hospital, outpatient facility, specialty clinic, or physician's office;
e) a pharmaceutical company; or
f) a diagnostic company;
which uses or pays for the method of claim 14.

19. A medical record of an individual patient comprising:
a) diagnostic evaluation determining development or existence of a chronic disease or disorder; and
b) theragnostic evaluation of the patient determining therapeutic appropriateness, therapeutic guidance, therapeutic effectiveness, and selection of alternative therapeutic strategy, the evaluation leading to selection of a treatment strategy.

20. A database comprising a plurality of medical records of claim 19, wherein:
a) a large majority of the medical records in the database include treatment response data; or
b) in some medical records the treatment strategy is complete and the patient has achieved remission or excellent response.

21. An entity selected from:
a) a pharmacy;
b) a pharmacy benefits management entity;
c) an employer or insurance entity, including a health insurer, commercial health insurer, or public/government health insurer;
d) a healthcare provider entity, including a clinic, hospital, outpatient facility, specialty clinic, or physician's office;
e) a pharmaceutical company; or
f) a diagnostic company;
which uses or possesses the database of claim 20.
